(19) United States
(12) Patent Application Publication
(10) Pub. No.: US 2026/0206484 A1
(43) Pub. Date: Jul. 16, 2026

(54) COMPOUND FOR ORGANIC ELECTRONIC DEVICE, ORGANIC ELECTRONIC DEVICE USING THE SAME, AND ELECTRONIC APPARATUS THEREOF

(71) Applicant: DUK SAN NEOLUX CO., LTD., Cheonan-si (KR)

(72) Inventors: Yeong Ran SONG, Cheonan-si (KR); Jung Geun LEE, Cheonan-si (KR)

(73) Assignee: DUK SAN NEOLUX CO., LTD., Cheonan-si (KR)

(21) Appl. No.: 19/410,818

(22) Filed: Dec. 5, 2025

(30) Foreign Application Priority Data
Dec. 6, 2024 (KR) 10-2024-0180495

(51) Int. Cl. H10K 85/60; C07B 59/00; C07C 211/54; C07D 307/91; C07D 333/76; C07F 7/08; H10K 50/11; H10K 85/40

(52) U.S. Cl. CPC H10K 85/633; C07B 59/001; C07C 211/54; C07D 307/91; C07D 333/76; C07F 7/081; H10K 85/636; C07C 2603/18; H10K 50/11; H10K 85/40; H10K 85/622; H10K 85/626; H10K 85/6574; H10K 85/6576

(57) ABSTRACT

Provided are a compound represented by Formula 1, an organic electronic device including a first electrode, a second electrode, and an organic layer between the first electrode and the second electrode, and an electronic apparatus including the organic electronic device, where the organic layer includes the compound represented by Formula 1 and thereby the driving voltage of the organic electronic device can be lowered, and the luminous efficiency and lifetime can be improved.

### TECHNICAL FIELD

The present invention relates to a compound for an organic electronic device, an organic electronic device using the same, and an electronic apparatus thereof.

### BACKGROUND

Generally, organic luminescence refers to the phenomenon of converting electrical energy into light energy using organic materials. An organic electronic device that utilizes the organic light-emitting phenomenon typically has a structure that includes an anode, a cathode, and an organic layer between them. Here, the organic layer is often composed of a multilayer structure consisting of different materials to increase the efficiency and stability of the organic electronic device, and may be composed of, for example, a hole injection layer, a hole transport layer, a light-emitting layer, an electron transport layer, and an electron injection layer.

The biggest issues with an organic electroluminescent device are its lifetime and efficiency, and as the display size increases (or as displays become large-area), these efficiency and lifetime problems must be solved.

Efficiency, lifetime, driving voltage, and the like are correlated with each other. If efficiency is increased, then the driving voltage is relatively lower, and as the driving voltage is lowered, the crystallization of the organic material due to Joule heating generated during operation is reduced, resulting in a tendency for the lifetime to increase.

However, efficiency cannot be maximized only by simply improving the organic layer. This is because long lifetime and high efficiency can be simultaneously achieved when the energy levels and T₁ values of the respective layers included in the organic layer, inherent material properties (mobility, interfacial properties, etc.) and the like form an optimal combination.

Therefore, in order to fully demonstrate the excellent characteristics of an organic electronic device, it is necessary to develop materials that form the organic layer of the device, especially a light-emitting auxiliary layer.

### DETAILED DESCRIPTION OF THE INVENTION

### Technical Problem

The present invention aims to provide a compound for an organic electronic device, which can lower the driving voltage of the device and improve the luminous efficiency and lifetime of the device, an organic electronic device using the same, and an electronic apparatus thereof.

### Technical Solution

In one aspect, the present invention provides a compound represented by the following Formula.

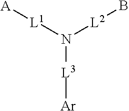

In another aspect, the present invention provides an organic electronic device and an electronic apparatus thereof, comprising the compound represented by the Formula.

In yet another aspect, the present invention provides a method for recovering the compound represented by the Formula.

### Advantageous Effects

By using the compound according to an embodiment of the present invention as a material for an organic electronic device, the driving voltage of the device can be lowered, and the luminous efficiency and lifetime can be improved.

### BRIEF DESCRIPTION OF THE DRAWINGS

FIGS. 1 to 3 are exemplary diagrams of an organic electroluminescent device according to an embodiment of the present invention.

### DETAILED DESCRIPTION OF THE EMBODIMENTS

In the present specification, “aryl group”, “arylene group”, and “aromatic ring” mean a hydrocarbon aromatic ring group, each having a carbon number of 6 to 60 unless otherwise described, and are not limited thereto. In the present invention, the aryl group or the arylene group comprises a monocyclic ring, a polycyclic ring, a condensed ring, and the like.

Further, the aryl group or the arylene group in the present specification may be an aryl group or an arylene group such as, for example, a C₆-C₆₀, a C₆-C₃₀, a C₆-C₂₉, a C₆-C₂₈, a C₆-C₂₇, a C₆-C₂₆, a C₆-C₂₅, a C₆-C₂₄, a C₆-C₂₃, a C₆-C₂₂, a C₆-C₂₁, a C₆-C₂₀, a C₆-C₁₉, a C₆-C₁₈, a C₆-C₁₇, a C₆-C₁₆, a C₆-C₁₅, a C₆-C₁₄, a C₆-C₁₃, a C₆-C₁₂, a C₆-C₁₁, a C₆-C₁₀, a C₆, a C₁₀, a C₁₂, a C₁₃, a C₁₄, a C₁₅, a C₁₆, a C₁₇, or a C₁₈ arylene group and may be specifically phenyl, biphenyl, naphthyl, terphenyl, phenanthrene, benzophenanthrene, triphenylene, chrysene, or a combination thereof, and particularly phenyl, naphthyl, phenanthrene, or a combination thereof, and particularly phenyl, naphthyl, phenanthrene, or a combination thereof.

In the present specification, the “fluorenyl group” means a substituted or unsubstituted fluorenyl group, and the “fluorenylene group” means a substituted or unsubstituted fluorenylene group. The fluorenyl group or fluorenylene group used in the present invention includes a spiro compound formed by the mutual bonding of R and R′ in the following structure, and also includes a compound in which adjacent R″s combine with each other to form a ring. The “substituted fluorenyl group” and the “substituted fluorenylene group” mean that at least one of R, R′, and R″ in the following structure is a substituent other than hydrogen, and R″ may range from 1 to 8 in the Formula below. In the present specification, the fluorenyl group, fluorenylene group, and the like may be described as a fluorene group or fluorene regardless of the valence.

Further, the fluorenyl group may be 9,9-dimethyl-9H-fluorene, 9,9-diphenyl-9H-fluorene, 9,9′-spirobifluorene, spiro[benzo[b]fluorene-11,9′-fluorene], benzo[b]fluorene, 11,11-diphenyl-11H-benzo[b]fluorene, 9-(naphthalen-2-yl)9-phenyl-9H-fluorene, and the like.

Further, when one R″ is substituted in the following Formula, it may be 1-fluorene, 2-fluorene, 3-fluorene, or 4-fluorene depending on the position where R″ is substituted, and when two R″s are substituted, the two R″s may be substituted at the 2^{nd} and 5^{th}, 2^{nd} and 7^{th}, 4^{th} and 5^{th}, 4^{th} and 7^{th} positions, and the like.

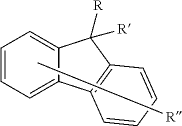

In the present specification, a “spiro compound” possesses a ‘spiro linkage’, and a spiro linkage refers to a linkage formed by two rings sharing only one common atom. The atom shared by the two rings is called the ‘spiro atom’, and these compounds are referred to as ‘monospiro-’, ‘dispiro-’, or ‘trispiro-’ compounds, respectively, depending on the number of spiro atoms contained in one compound. For example, when R and R″ combine with each other to form a ring in the fluorene structural formula above, a spiro compound may be formed. The formed ring may be a 5-membered, 6-membered, 10-membered, 13-membered, 14-membered, 17-membered, 18-membered, 21-membered, or 22-membered ring, including the spiro atom. Specifically, rings such as fluorene, benzofluorene, dibenzofluorene, xanthene, or thioxanthene may be formed. For example, when R and R″ combine with each other bonded to form a fluorene ring, the compound 9,9′-spirobifluorene is formed.

In the present specification, a “heterocyclic group” includes not only aromatic heterocyclic rings such as a “heteroaryl group” or a “heteroarylene group” but also non-aromatic heterocyclic rings. Unless otherwise described, the term refers to a ring having 2 to 60 carbon atoms and including one or more heteroatoms, but is not limited thereto. Further, a “heteroatom” refers to an element other than carbon, such as, for example, N, O, S, P or Si and may include a heteroatom group such as SO₂ or P═O instead of the carbon atom forming the ring, as in the following compounds.

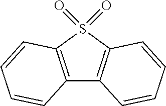 [SMILES: O=S1(=O)c2ccccc2-c2ccccc21]

Furthermore, the heterocyclic group includes a monocyclic ring, a polycyclic ring, or a condensed ring containing a heteroatom. In the case of a condensed ring, it is defined as corresponding to a heterocyclic ring if at least one of the condensed rings is a ring containing a heteroatom. For example, a condensed ring formed by the condensation of a heterocyclic ring such as furan, dihydrofuran, thiophene, pyrrole, or pyridine and an aromatic ring such as benzene, naphthalene, or phenanthrene, or an aliphatic ring such as cyclopentane or cyclohexane, also corresponds to a heterocyclic ring. Additionally, in the case of a spiro compound, it also corresponds to a heterocyclic ring if a heteroatom is included in at least one ring.

Furthermore, the heterocyclic group in the present specification may be a heterocyclic group such as, for example, a C₂-C₆₀, a C₂-C₃₀, a C₂-C₂₉, a C₂-C₂₈, a C₂-C₂₇, a C₂-C₂₆, a C₂-C₂₅, a C₂-C₂₄, a C₂-C₂₃, a C₂-C₂₂, a C₂-C₂₁, a C₂-C₂₀, a C₂-C₁₉, a C₂-C₁₈, a C₂-C₁₇, a C₂-C₁₆, a C₂-C₁₅, a C₂-C₁₄, a C₂-C₁₃, a C₂-C₁₂, a C₂-C₁₁, a C₂-C₁₀, a C₂-C₉, a C₂-C₈, a C₂-C₇, a C₂-C₆, a C₂-C₅, a C₂-C₄, a C₂-C₃, a C₂, a C₃, a C₄, a C₅, a C₆, a C₇, a C₈, a C₉, a C₁₀, a C₁₁, a C₁₂, a C₁₃, a C₁₄, a C₁₅, a C₁₆, a C₁₇, a C₁₈, a C₁₉, C₂₀, a C₂₁, a C₂₂, a C₂₃, a C₂₄, a C₂₅, a C₂₆, a C₂₇, a C₂₈, or a C₂₉ heterocyclic group, and may be specifically pyridine, pyrimidine, pyrazine, pyridazine, triazine, pyrrole, indole, phenyl-indole, benzindole, phenyl-benzindole, pyrazinoindole, quinoline, isoquinoline, benzoquinoline, pyridoquinoline, quinazoline, benzoquinazoline, dibenzoquinazoline, phenanthroquinazoline, quinoxaline, benzoquinoxaline, dibenzoquinoxaline, furan, benzofuran, naphthobenzofuran, dibenzofuran, dinaphthofuran, phenanthrobenzofuran, thiophene, benzothiophene, dibenzothiophene, naphthobenzothiophene, dinaphthothiophene, phenanthrobenzothiophene, carbazole, phenyl-carbazole, benzocarbazole, phenyl-benzocarbazole, naphthyl-benzocarbazole, dibenzocarbazole, indolocarbazole, benzofuropyridine, benzothienopyridine, benzofuropyridine, benzothienopyrimidine, benzothienopyrazine, benzofuropyrazine, benzimidazole, benzothiazole, benzosilole, dibenzosilole, phenanthroline, dihydro-phenylphenazine, 10-phenyl-10H-phenoxazine, phenoxazine, phenothiazine, dibenzodioxin, benzodibenzodioxin, thianthrene, oxazole, benzoxazole, naphthoxazole, phenanthrooxazole, dibenzothienobenzoxazole, dibenzofurobenzoxazole, 9,9-dimethyl-9H-xanthene, 9,9-dimethyl-9H-thioxanthene, dihydrophenyldimethylacridine, spiro[fluorene-9,9′-xanthene], and the like.

In the present specification, an “alicyclic group” means a cyclic hydrocarbon excluding an aromatic hydrocarbon, and includes a monocyclic ring, a polycyclic ring, a condensed ring, a spiro compound, and the like. Unless otherwise described, it means a ring having 3 to 60 carbon atoms, but is not limited thereto. Specifically, the alicyclic (ring) group in the present specification is defined as a hydrocarbon ring that does not include an aromatic ring. Therefore, not only a saturated hydrocarbon ring such as a cycloalkyl group but also a ring having one or more double bonds in the ring is considered to correspond to an alicyclic group as long as it is not an aromatic hydrocarbon.

Furthermore, the alicyclic group in the present specification may be an alicyclic group such as, for example, a C₃-C₆₀, a C₃-C₃₀, a C₃-C₂₉, a C₃-C₂₈, a C₃-C₂₇, a C₃-C₂₆, a C₃-C₂₅, a C₃-C₂₄, a C₃-C₂₃, a C₃-C₂₂, a C₃-C₂₁, a C₃-C₂₀, a C₃-C₁₉, a C₃-C₁₈, a C₃-C₁₇, a C₃-C₁₆, a C₃-C₁₅, a C₃-C₁₄, a C₃-C₁₃, a C₃-C₁₂, a C₃-C₁₁, a C₃-C₁₀, a C₃-C₈, a C₃-C₆, a C₆, a C₁₀, a C₁₁, a C₁₂, a C₁₃, a C₁₄, a C₁₅, a C₁₆, a C₁₇, or a C₁₈ alicyclic group. Furthermore, the alicyclic group may be a cycloalkyl group, such as, for example, cyclobutane, cyclopentane, cyclohexane, cyclohexene, bicycloheptane, adamantyl, and the like.

In the present specification, a “fused ring (group)” or “condensed ring (group)” means a ring in which an alicyclic group and an aromatic hydrocarbon (aromatic ring group or aryl ring) combine with each other condensed, unless otherwise described. Unless otherwise described, it means a ring in which an alicyclic group having 3 to 60 carbon atoms and an aromatic hydrocarbon having 6 to 60 carbon atoms combine with each other condensed.

In the present specification, an “alkyl group” includes a normal (straight-chain) alkyl group, a branched (side-chain) alkyl group, and the like. The alkyl group may be an alkyl group such as, for example, a C₁-C₂₀, a C₁-C₁₀, a C₁-C₉, a C₁-C₈, a C₁-C₇, a C₁-C₆, a C₁-C₈, a C₁-C₄, a C₁-C₃, a C₁-C₂, a C₁, a C₂, a C₃, a C₄, a C₅, a C₆, or a C₇ alkyl group, and may be, for example, a methyl group, ethyl group, propyl group, isopropyl group, butyl group, t-butyl group, or pentyl group, and furthermore, the hydrogen atoms of the alkyl group may be substituted with deuterium.

In the present specification, the ‘group name’ corresponding to the aryl group, arylene group, heterocyclic group, and the like, exemplified as examples of each symbol and its substituent, may be described as the ‘name of the group reflecting the valence’ but may also be described as the ‘name of the parent compound’. For example, in the case of phenanthrene, which is a type of aryl group, the name of the group may be described by distinguishing the valences, such as ‘phenanthryl’ for a monovalent group and ‘phenanthrylene’ for a divalent group. However, it may also be described as the ‘parent compound name’, ‘phenanthrene’, regardless of the valence. Similarly, in the case of pyrimidine, it may be described as ‘pyrimidine’ regardless of the valence, or it may be described as the ‘name of the group’ corresponding to the respective valence, such as pyrimidyl group for a monovalent group or pyrimidylene group for a divalent group.

Furthermore, in the present specification, numbers or alphabets indicating position (locants), and the like, may be omitted when describing the name of a compound or the name of a substituent. For example, pyrido[4,3-d]pyrimidine may be described as pyridopyrimidine, benzofuro[2,3-d]pyrimidine as benzofuropyrimidine, 9,9-dimethyl-9H-fluorene as dimethylfluorene, and the like. Accordingly, both benzo[g]quinoxaline and benzo[f]quinoxaline may be described as benzoquinoxaline.

Furthermore, unless otherwise explicitly described, the Formula s used in the present specification shall be applied identically to the substituent definitions based on the index definitions of the following Formulas.

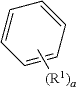

Here, when a is an integer of 0, it means that the substituent R¹ is non-existent. That is, when a is 0, it means that all carbons forming the benzene ring are bonded to hydrogen, in which case, the indication of hydrogen bonded to carbon may be omitted and the Formula or compound may be described. Further, when a is an integer of 1, one substituent R¹ is bonded to any one carbon among the carbons forming the benzene ring. When a is an integer of 2 or 3, it may be bonded as shown below, for example, and even when a is an integer of 4 to 6, it is bonded to the carbon of the benzene ring in a similar manner. When a is an integer of 2 or more, the R¹ substituents may be the same as or different from each other.

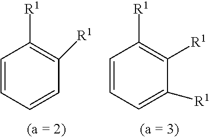

Furthermore, in the present specification, a ring means an aryl ring, a heteroaryl ring, a fluorene ring, an alicyclic group, a fused ring, and the like. Terms such as ‘number-ring’, ‘number-condensed ring’, or ‘number-fused ring’ mean a form in which the number of rings corresponding to the number combine with each other fused, and a ‘number-membered ring’ may mean the shape of the ring. For example, naphthalene corresponds to a 2-ring, a 2-condensed ring, or a 2-fused ring, and anthracene corresponds to a 3-ring, a 3-condensed ring, or a 3-fused ring. Thiophene or furan, and the like, correspond to a 5-membered heterocyclic ring, and benzene or pyridine corresponds to a 6-membered aromatic ring.

Furthermore, in the present specification, a ring formed by the mutual bonding of adjacent groups may be selected from the group consisting of a C₆-C₆₀ aromatic group; a fluorenyl group; a C₂-C₆₀ heterocyclic group comprising at least one heteroatom selected from O, N, S, Si and P; a C₃-C₆₀ alicyclic group; and a fused ring group of a C₃-C₆₀ alicyclic group and a C₆-C₆₀ aromatic group. Here, the aromatic group may be an aryl ring, and the heterocyclic group may include a heteroaryl ring.

In the present specification, ‘adjacent groups’ means, by way of example with the following Formula, not only R₁ and R₂, R₂ and R₃, R₃ and R₄, R₅ and R₆, but also R₇ and R₈ which share a single carbon atom. Furthermore, it may also include substituents bonded to non-adjacent ring-constituting elements (such as carbon or nitrogen), such as R₁ and R₇, R₁ and R₈, or R₄ and R₅. That is, if a substituent is present on a ring-constituting element (such as carbon or nitrogen) at an immediately adjacent position, those may become adjacent groups. However, if no substituent is bonded to the ring-constituting element at an immediately adjacent position, the substituent bonded to the next ring-constituting element may become a neighboring group. Additionally, substituents bonded to the same ring-constituting carbon may also be referred to as adjacent groups. When substituents bonded to the same carbon, such as R₇ and R₈ in the Formula below, combine with each other to form a ring, a compound including a spiro moiety may be formed.

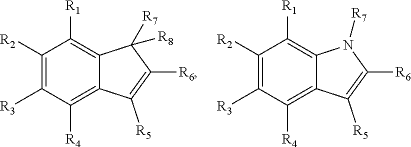

In addition, the expression adjacent (neighboring) groups may combine with each other to form a ring’ is used interchangeably with the expression ‘adjacent groups optionally combine with each other to form a ring’, and means the case where at least one pair of adjacent groups combine with each other to form a ring.

In addition, the expression ‘adjacent groups may combine with each other to form a ring’ as used herein means that adjacent groups can consequently form a ring, and it is not premised on R¹ and R² being substituents containing an unsaturated bond, such as an alkenyl or alkynyl group.

When adjacent groups combine with each other to form a ring, the ring may be selected from the group consisting of a C₆-C₆₀ aromatic group (an aryl ring); a fluorenyl group; a C₃-C₆₀ alicyclic group; a C₂-C₆₀ heterocyclic group comprising at least one heteroatom selected from O, N, S, Si and P; and a fused ring group of a C₃-C₆₀ alicyclic group and a C₆-C₆₀ aromatic group.

When adjacent groups combine with each other to form an aromatic ring (aryl group), the aromatic ring may be, for example, a C₆-C₂₀, a C₆-C₁₈, a C₆-C₁₆, a C₆-C₁₄, a C₆-C₁₃, a C₆-C₁₂, a C₆-C₁₀, a C₆, a C₁₀, a C₁₂, a C₁₄, a C₁₅, a C₁₆, or a C₁₈ aromatic ring, and specifically, it may be benzene, naphthalene, anthracene, phenanthrene, pyrene, or triphenylene, and the like.

In addition, substituents such as an aryl group, an arylene group, a fluorenyl group, a fluorenylene group, a heterocyclic group, an alicyclic group, a fused ring group, an alkyl group, an alkenyl group, an alkynyl group, an alkoxy group, an aryloxy group, an alkylthio group, an arylthio group, and a ring formed by adjacent groups combining with each other, may each be substituted with one or more substituents selected from the group consisting of deuterium; halogen; a silane group substituted or unsubstituted with a C₁-C₂₀ alkyl group or a C₆-C₂₀ aryl group; a phosphine oxide substituted or unsubstituted with a C₁-C₂₀ alkyl group or a C₆-C₂₀ aryl group; a cyano group; a C₁-C₂₀ alkoxy group; a C₆-C₃₀ aryloxy group; a C₁-C₂₀ alkyl group; a C₂-C₂₀ alkenyl group; a C₂-C₂₀ alkynyl group; a C₆-C₃₀ aryl group; a C₆-C₃₀ aryl group substituted with deuterium; a C₃-C₃₀ alicyclic group; a fused ring group of a C₆-C₃₀ aromatic ring and a C₃-C₃₀ alicyclic group; and a C₂-C₃₀ heterocyclic group containing at least one heteroatom selected from O, N, S, Si, and P, and neighboring said substituents may combine with each other to form a ring, and the hydrogen of said substituents may be replaced by deuterium.

In addition, when at least one of an aryl group, an arylene group, a fluorenyl group, a fluorenylene group, a heterocyclic group, an alicyclic group, a fused ring group, an alkyl group, an alkenyl group, an alkynyl group, an alkoxy group, an aryloxy group, an alkylthio group, an arylthio group, and a ring formed by adjacent groups combining with each other is substituted with an aryl group, the aryl group may be, for example, a C₆-C₃₀, a C₆-C₂₉, a C₆-C₂₈, a C₆-C₂₇, a C₆-C₂₆, a C₆-C₂₅, a C₆-C₂₄, a C₆-C₂₃, a C₆-C₂₂, a C₆-C₂₁, a C₆-C₂₀, a C₆-C₁₉, a C₆-C₁₈, a C₆-C₁₇, a C₆-C₁₆, a C₆-C₁₅, a C₆-C₁₄, a C₆-C₁₃, a C₆-C₁₂, a C₆-C₁₁, a C₆-C₁₀, a C₆, a C₁₀, a C₁₂, a C₁₃, a C₁₄, a C₁₅, a C₁₆, a C₁₇, or a C₁₈ aryl group, and the like, and specifically, may be phenyl, biphenyl, naphthyl, terphenyl, phenanthrenyl, benzophenanthrenyl, triphenylenyl, chrysenyl, or a combination thereof, particularly phenyl, naphthyl, or a combination thereof.

In addition, when at least one of an aryl group, an arylene group, a fluorenyl group, a fluorenylene group, a heterocyclic group, an alicyclic group, a fused ring group, an alkyl group, an alkenyl group, an alkynyl group, an alkoxy group, an aryloxy group, an alkylthio group, an arylthio group, and a ring formed by adjacent groups combining with each other is substituted with a heterocyclic group, the heterocyclic group may be, for example, a C₂-C₃₀, a C₂-C₂₉, a C₂-C₂₈, a C₂-C₂₇, a C₂-C₂₆, a C₂-C₂₅, a C₂-C₂₄, a C₂-C₂₃, a C₂-C₂₂, a C₂-C₂₁, a C₂-C₂₀, a C₂-C₁₉, a C₂-C₁₈, a C₂-C₁₇, a C₂-C₁₆, a C₂-C₁₅, a C₂-C₁₄, a C₂-C₁₃, a C₂-C₁₂, a C₂-C₁₁, a C₂-C₁₀, a C₂-C₉, a C₂-C₈, a C₂-C₇, a C₂-C₆, a C₂-C₅, a C₂-C₄, a C₂-C₃, a C₂, a C₃, a C₄, a C₅, a C₆, a C₇, a C₈, a C₉, a C₁₀, a C₁₁, a C₁₂, a C₁₃, a C₁₄, a C₁₅, a C₁₆, a C₁₇, a C₁₈, a C₁₉, or a C₂₀ heterocyclic group.

In addition, when at least one of an aryl group, an arylene group, a fluorenyl group, a fluorenylene group, a heterocyclic group, an alicyclic group, a fused ring group, an alkyl group, an alkenyl group, an alkynyl group, an alkoxy group, an aryloxy group, an alkylthio group, an arylthio group, and a ring formed by adjacent groups combining with each other is substituted with an alicyclic group, the alicyclic group may be, for example, a C₃-C₃₀, a C₃-C₂₉, a C₃-C₂₈, a C₃-C₂₇, a C₃-C₂₆, a C₃-C₂₅, a C₃-C₂₄, a C₃-C₂₃, a C₃-C₂₂, a C₃-C₂₁, a C₃-C₂₀, a C₃-C₁₉, a C₃-C₁₈, a C₃-C₁₇, a C₃-C₁₆, a C₃-C₁₅, a C₃-C₁₄, a C₃-C₁₃, a C₃-C₁₂, a C₃-C₁₁, a C₃-C₁₀, a C₃-C₈, a C₃-C₆, a C₆, a C₁₀, a C₁₁, a C₁₂, a C₁₃, a C₁₄, a C₁₅, a C₁₆, a C₁₇, or a C₁₈ alicyclic group.

In addition, when at least one of an aryl group, an arylene group, a fluorenyl group, a fluorenylene group, a heterocyclic group, an alicyclic group, a fused ring group, an alkyl group, an alkenyl group, an alkynyl group, an alkoxy group, an aryloxy group, an alkylthio group, an arylthio group, and a ring formed by adjacent groups combining with each other is substituted with an alkyl group, the alkyl group may be, for example, a C₁-C₂₀, a C₁-C₁₀, a C₁-C₉, a C₁-C₈, a C₁-C₇, a C₁-C₆, a C₁-C₅, a C₁-C₄, a C₁-C₃, a C₁-C₂, a C₁, a C₂, a C₃, a C₄, a C₅, a C₆, or a C₇ alkyl group, may be, for example, a methyl group, an ethyl group, a propyl group, an isopropyl group, a butyl group, a t-butyl group, a pentyl group, and the like, and the hydrogen of the alkyl group may be replaced by deuterium.

Unless otherwise specified herein, the asterisk (*) or

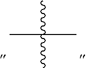

indicates a bonding site.

Hereinafter, the stacked structure of the organic electronic device including the compound of the present invention will be described with reference to FIGS. 1 to 3.

It should be noted that in adding reference numerals to the components in each drawing, the same components are assigned the same numerals as much as possible, even if they are indicated in different drawings. Also, in describing the present invention, when it is determined that a detailed description of related known configurations or functions may obscure the gist of the present invention, the detailed description thereof will be omitted.

In describing the constituent elements of the present invention, terms such as first, second, A, B, (a), (b), and the like may be used. These terms are merely for the purpose of distinguishing one constituent element from another, and the essence, order, or sequence of the corresponding constituent elements is not limited by the terms. When a constituent element is described as being “connected,” “coupled,” or “joined” to another constituent element, it should be understood that the constituent element may be directly connected or joined to the other constituent element, or there may be yet another constituent element “connected,” “coupled,” or “joined” between the constituent elements.

In addition, when a constituent element such as a layer, film, region, or plate is described as being “on” or “above” another constituent element, it should be understood that this includes not only the case where it is directly above the other constituent element, but also the case where another constituent element is interposed in between. Conversely, when a constituent element is described as being “directly on” or “immediately above” another part, it should be understood to mean that no other part is interposed in between.

FIGS. 1 to 3 respectively illustrate examples of an organic electronic device according to embodiments of the present invention.

Referring to FIG. 1, an organic electronic device 100 according to an embodiment of the present invention comprises a first electrode 110, a second electrode 170, and an organic layer formed between the first electrode 110 and the second electrode 170 on a substrate (not shown), and an inorganic layer may also be present between the first electrode 110 and the second electrode 170.

For example, the first electrode 110 may be an anode (positive electrode), and the second electrode 170 may be a cathode (negative electrode). In an inverted organic electronic device, however, the first electrode may be a cathode, while the second electrode may be an anode.

The organic layer refers to a layer comprising at least one organic material. For example, the organic layer may include a hole injection layer 120, a hole transport layer 130, a light-emitting layer 140, an electron transport layer 150, and an electron injection layer 160. However, the electron injection layer 160 may be an inorganic layer that does not contain any organic material.

Specifically, a hole injection layer 120, a hole transport layer 130, a light-emitting layer 140, an electron transport layer 150, and an electron injection layer 160 may be sequentially formed on the first electrode 110.

Preferably, a layer for improving the luminous efficiency 180 may be formed on one side of either the first electrode 110 or the second electrode 170, which does not face the organic layer or the inorganic layer. When the layer for improving the luminous efficiency 180 is formed, the luminous efficiency of the organic electronic device can be enhanced.

For example, when the layer for improving the luminous efficiency 180 may be formed on the second electrode 170, in the case of a top-emission organic electroluminescent device, optical energy loss due to surface plasmon polaritons (SPPs) at the second electrode 170 may be reduced, and in the case of a bottom-emission organic electroluminescent device, the layer for improving the luminous efficiency 180 may function as a buffer layer for the second electrode 170.

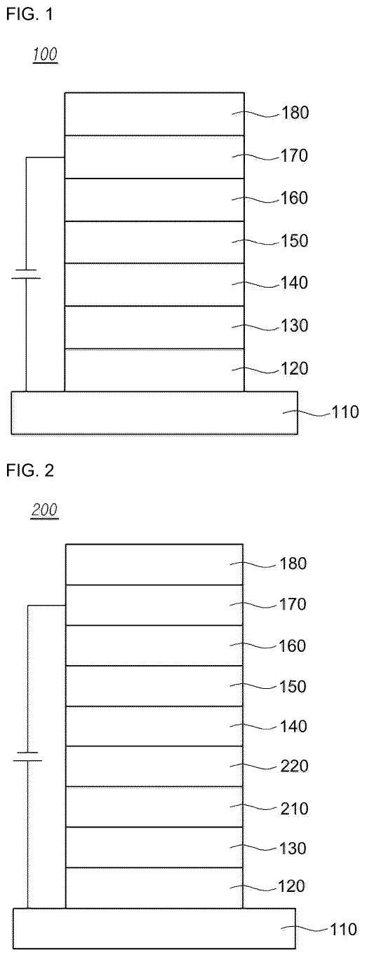

A buffer layer 210 or a light-emitting auxiliary layer 220 may additionally be formed between the hole transport layer 130 and the light-emitting layer 140, as will be described with reference to FIG. 2.

Referring to FIG. 2, an organic electronic device 200 according to another embodiment of the present invention may sequentially include a hole injection layer 120, a hole transport layer 130, a buffer layer 210, a light-emitting auxiliary layer 220, a light-emitting layer 140, an electron transport layer 150, an electron injection layer 160, and a second electrode 170 on a first electrode 110, and a layer for improving the luminous efficiency 180 may be formed on the second electrode.

Although not illustrated in FIG. 2, an electron transport auxiliary layer may additionally be formed between the light-emitting layer 140 and the electron transport layer 150.

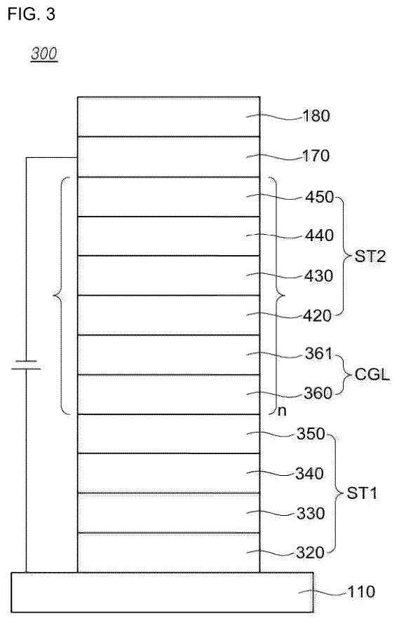

In addition, according to another embodiment of the present invention, an organic layer may be in the form of a plurality of stacks, each including a hole transport layer, a light-emitting layer, and an electron transport layer. This will be described with reference to FIG. 3.

Referring to FIG. 3, an organic electronic device 300 according to another embodiment of the present invention may include two or more sets of stacks (ST1, ST2) of organic layers formed in multiple layers between the first electrode 110 and the second electrode 170, and a charge generation layer (CGL) may be formed between the stacks of the organic layers.

Specifically, the organic electronic device according to the embodiment of the present invention may comprise a first electrode 110, a first stack ST1, a charge generation layer CGL, a second stack ST2, and a second electrode 170 and a layer for improving light efficiency 180.

The first stack ST1 is an organic layer formed on the first electrode 110, and may comprise a first hole injection layer 320, a first hole transport layer 330, a first light-emitting layer 340, and a first electron transport layer 350. The second stack ST2 may comprise a second hole injection layer 420, a second hole transport layer 430, a second light-emitting layer 440, and a second electron transport layer 450. As such, the first stack and the second stack may have the same or different stacked structures of organic layers.

The charge generation layer CGL may be formed between the first stack ST1 and the second stack ST2. The charge generation layer CGL may comprise a first charge generation layer 360 and a second charge generation layer 361. It is formed between the first light-emitting layer 340 and the second light-emitting layer 440 to enhance the current efficiency of each light-emitting layer and facilitate charge distribution.

The first light-emitting layer 340 may comprise an emissive material that comprises a blue host doped with a blue fluorescent dopant, and the second light-emitting layer 440 may comprise an emissive material that comprises a green host doped with both a greenish-yellow dopant and a red dopant. However, the materials of the first light-emitting layer 340 and the second light-emitting layer 440 according to an embodiment of the present invention are not limited thereto.

In FIG. 3, n may be an integer from 1 to 5, and when n is 2, a charge generation layer (CGL) and a third stack may be additionally formed on the second stack ST2.

When a plurality of light-emitting layers are formed in a multi-layer stack structure as shown in FIG. 3, it is possible to manufacture an organic electroluminescent device that emits not only white light but also various colors, where the white light is produced by the mixing of light emitted from each light-emitting layer.

Compound represented by Formula 1 of the present invention may be included in an organic layer. For example, the compound represented by Formula 1 of the present invention can be used as a material for a hole injection layer 120, 320, 420, a hole transport layer 130, 330, 430, a buffer layer 210, a light-emitting auxiliary layer 220, an electron transport layer 150, 350, 450, a light-emitting layer 140, 340, 440, and/or a light efficiency improving layer 180, more preferably, as material of a light-emitting auxiliary layer 220.

Even if the cores of the compounds are identical or similar, their band gaps, electronic properties, and interfacial characteristics may vary depending on which substituents are bonded and at which positions. Therefore, it is necessary to study the selection of the core structure and the combination with sub-substituents attached to the core. In particular, both long lifetime and high efficiency can be achieved simultaneously when the optimal combination of energy levels, T₁ values, and intrinsic material properties (such as mobility and interfacial characteristics) is realized among the layers of the organic structure.

Therefore, by using the compound represented by Formula 1 as material of a light-emitting auxiliary layer 220, it is possible to optimize the energy levels and T₁ values, intrinsic material properties (such as mobility and interfacial characteristics) between the respective layers of the organic layer, as a result, the lifetime and efficiency of an organic electronic device can be simultaneously improved.

The organic light-emitting device according to an embodiment of the present invention may be fabricated using various deposition methods, comprising physical vapor deposition (PVD) or chemical vapor deposition (CVD). For example, the organic electronic device may be manufactured by forming the anode 110 on the substrate by depositing a metal, a conductive metal oxide, or a mixture thereof, then forming an organic layer comprising the hole injection layer 120, the hole transport layer 130, the light-emitting layer 140, the electron transport layer 150, and the electron injection layer 160 thereon, and finally depositing a material that can be used as the cathode 170. In addition, a light-emitting auxiliary layer 220 may be formed between the hole transport layer 130 and the light-emitting layer 140, and an electron transport auxiliary layer (not shown) may additionally be formed between the light-emitting layer 140 and the electron transport layer 150. As described above, the organic layer may be formed in a stacked structure.

In addition, the organic layer may be manufactured with fewer layers by using various polymer materials through a solution process or solvent-based process, such as spin coating, nozzle printing, inkjet printing, slot coating, dip coating, roll-to-roll, doctor blading, screen printing, or thermal transfer, instead of deposition. Since the organic layer according to the present invention may be formed in various ways, the scope of protection of the present invention is not limited by the method of forming the organic layer.

The organic electronic device according to an embodiment of the present invention may be a top-emission type, a bottom-emission type, or a dual-emission type, depending on the materials used.

In addition, the organic electronic device according to an embodiment of the present invention may be selected from the group consisting of an organic electroluminescent device, an organic solar cell, an organic photoconductor, an organic transistor, a monochromatic illumination device, and a quantum dot display device.

Another embodiment of the present invention provides an electronic apparatus comprising a display device including the above-described organic electronic device and a control unit for controlling the display device. The electronic apparatus may be a wired or wireless communication terminal currently in use or to be developed in the future, and comprises all types of electronic devices, such as mobile communication terminals (e.g., cellular phones), navigation units, game console, various types of TVs, and computers.

Hereinafter, a compound according to an aspect of the present invention will be described.

The compound according to an aspect of the present invention is represented by the following Formula 1.

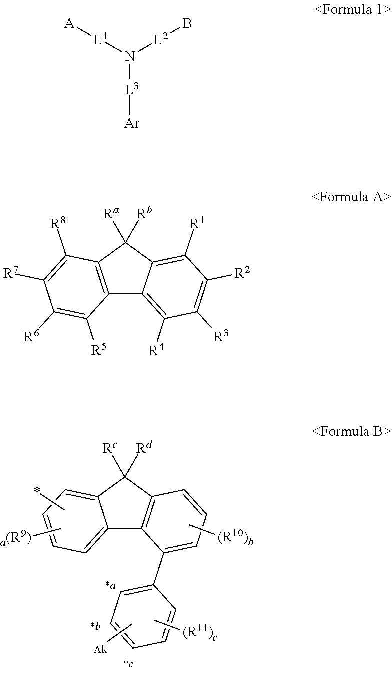

In the Formula 1, each symbol is defined as follows.

A is Formula A and is bonded to L¹ through one of R¹ to R⁸. That is, one of R¹ to R⁸ is a linking group (a single bond) connected to L¹.

B is Formula B and is bonded to L² through the asterisk (*).

Ar is selected from the group consisting of a C₆-C₆₀ aryl group, a fluorenyl group, C₂-C₆₀ heterocyclic group containing at least one heteroatom of O, N, S, Si and P, a C₃-C₆₀ alicyclic group, and a fused ring of a C₆-C₆₀ aromatic ring and a C₃-C₆₀ alicyclic ring.

L¹ to L³ are each independently selected from the group consisting of a sing bond, a C₆-C₆₀ arylene group, a fluorenylene group, a C₂-C₆₀ heterocyclic group containing at least one heteroatom of O, N, S, Si and P, a C₃-C₆₀ alicyclic group, and a fused ring of a C₆-C₆₀ aromatic ring and a C₃-C₆₀ alicyclic group.

R¹ to R¹¹, R^{a} to R^{d} are each independently selected from the group consisting of hydrogen, deuterium, halogen, a cyano group, a C₆-C₆₀ aryl group, a fluorenyl group, a C₂-C₆₀ heterocyclic group containing at least one heteroatom of O, N, S, Si and P, a C₃-C₆₀ alicyclic group, a fused ring of a C₆-C₆₀ aromatic ring and a C₃-C₆₀ alicyclic ring, a C₁-C₂₀ alkyl group, a C₂-C₂₀ alkenyl group, a C₂-C₂₀ alkynyl group, a C₁-C₂₀ alkoxyl group, and a C₆-C₆₀ aryloxy group, and adjacent groups may combine with each other to form a ring.

With the proviso that cases where R¹ to R⁸ are all hydrogen or deuterium are excluded. That is, at least one of R¹ to R⁸ is a substituent other than hydrogen and deuterium or adjacent groups combine with each other to form a ring.

When adjacent groups, for example, R¹ and R², R² and R³, R³ and R⁴, R⁴ and R⁵, R⁵ and R⁶, R⁶ and R⁷, R⁷ and R⁸, adjacent R⁹s, adjacent R¹⁰s, adjacent R¹¹s, R^{a} and R^{b}, R^{c} and R^{d} combine with each other to form a ring, the ring may be selected from the group consisting of a C₆-C₆₀ aromatic ring group, a fluorenylene group, a C₃-C₆₀ alicyclic group, a C₂-C₆₀ heterocyclic group containing at least one heteroatom of O, N, S, Si and P, and a fused ring of a C₃-C₆₀ alicyclic ring and a C₆-C₆₀ aromatic ring.

When an aromatic ring is formed by adjacent groups, the aromatic ring may be, for example, a C₆-C₂₀, a C₆-C₁₈, a C₆-C₁₆, a C₆-C₁₄, a C₆-C₁₃, a C₆-C₁₂, a C₆-C₁₀, a C₆, a C₁₀, a C₁₂, a C₁₄, a C₁₅, a C₁₆, or a C₁₈ aromatic ring, specifically, benzene, naphthalene, anthracene, phenanthrene, pyrene, triphenylene, etc.

When R^{a} and R^{b} combine with each other or R^{c} and R^{d} combine with each other to form a ring, a spiro compound may be formed.

Ak is a C₁-C₂₀ alkyl group or a C₃-C₆₀ alicyclic group, and Ak is bonded to any one position among *a, *b, and *c.

a and b are each an integer from 0 to 3, and c is an integer from 0 to 4. When these are integers of 2 or more, each of the plurality of R⁹, each of the plurality of R¹⁰, each of the plurality of R¹¹ are the same as or different from each other.

When at least one of R¹ to R¹¹, R^{a} to R^{d}, Ar is an aryl group, or at least one of L¹ to L³ is an arylene group, the aryl group or the arylene group may be, for example, a C₆-C₆₀, a C₆-C₃₀, a C₆-C₂₅, a C₆-C₂₀, a C₆-C₁₈, a C₆-C₁₆, a C₆-C₁₄, a C₆-C₁₂, a C₆-C₁₀, a C₆, a C₁₀, a C₁₂, a C₁₄, a C₁₅, a C₁₆, a C₁₈, a C₂₀, a C₂₅, a C₃₀ aryl group or arylene group, specifically, phenyl, biphenyl, naphthyl, terphenyl, phenanthrene, triphenylene, or a combination thereof, such as a combination of phenyl, naphthyl, and phenanthrenyl.

When at least one of R¹ to R¹¹, R^{a} to R^{d}, Ar, L¹ to L³ is a heterocyclic group, the heterocyclic group may be, for example, a C₂-C₆₀, a C₂-C₃₀, a C₂-C₂₅, a C₂-C₂₀, a C₂-C₁₆, a C₂-C₁₂, a C₂-C₁₀, a C₂-C₉, a C₂-C₈, a C₂-C₇, a C₂-C₆, a C₂-C₅, a C₂-C₄, a C₂-C₃, a C₂, a C₃, a C₄, a C₅, a C₆, a C₇, a C₈, a C₉, a C₁₀, a C₁₁, a C₁₂, a C₁₃, a C₁₄, a C₁₅, a C₁₆, a C₁₇, a C₁₈, a C₁₉, a C₂₀, a C₂₁, a C₂₂, a C₂₃, a C₂₄, a C₂₅, a C₂₆, a C₂₇, a C₂₈, or a C₂₉ heterocyclic group, specifically, pyridine, pyrimidine, pyrazine, triazine, indole, phenyl-indole, quinazoline, benzoquinazoline, quinoxaline, benzoquinoxaline, benzofuran, naphthobenzofuran, dibenzofuran, dinaphthofuran, phenanthrobenzofuran, thiophene, benzothiophene, dibenzothiophene, naphthobenzothiophene, dinaphthothiophene, phenanthrobenzothiophene, carbazole, phenyl-carbazole, benzocarbazole, phenyl-benzocarbazole, naphthyl-benzocarbazole, dibenzocarbazole, indolocarbazole, benzothienopyrimidine, benzofuropyrimidine, benzothienopyrazine, benzofuropyrazine, dibenzosilole, benzoxazole, naphthoxazole, phenanthroxazole, dibenzothienobenzoxazole, dibenzofurobenzoxazole, spiro[fluorene-9,9′-xanthene].

When at least one of R¹ to R¹¹, R^{a} to R^{d}, Ar, L¹ to L³, Ak is an alicyclic group, the alicyclic group may be, for example, a C₃-C₃₀, a C₃-C₂₉, a C₃-C₂₈, a C₃-C₂₇, a C₃-C₂₆, a C₃-C₂₅, a C₃-C₂₄, a C₃-C₂₃, a C₃-C₂₂, a C₃-C₂₁, a C₃-C₂₀, a C₃-C₁₉, a C₃-C₁₈, a C₃-C₁₇, a C₃-C₁₆, a C₃-C₁₅, a C₃-C₁₄, a C₃-C₁₃, a C₃-C₁₂, a C₃-C₁₁, a C₃-C₁₀, a C₃-C₈, a C₃-C₆, a C₆, a C₁₀, a C₁₁, a C₁₂, a C₁₃, a C₁₄, a C₁₅, a C₁₆, a C₁₇, or a C₁₈ alicyclic group, specifically, cyclobutane, cyclopentane, cyclohexane, cyclohexene, bicycloheptane, adamantyl, etc.

When at least one of R¹ to R¹¹, R^{a} to R^{d}, Ar is a fluorenyl group, or when at least one of L¹ to L³ is a fluorenylene group, the fluorenyl group or the fluorenylene group may be 9,9-dimethyl-9H-fluorene, 9,9-diphenyl-9H-fluorene, 9,9′-spirobifluorene, spiro[benzo[b]fluorene-11,9′-fluorene], benzo[b]fluorene, 11,11-diphenyl-11H-benzo[b]fluorene, or 9-(naphthalen-2-yl)-9-phenyl-9H-fluorene.

When at least one of R¹ to R¹¹, R^{a} to R^{d}, Ak is an alkyl group, the alkyl group may be, for example, a C₁-C₂₀, a C₁-C₁₀, a C₁-C₄, a C₁, a C₂, a C₃, or a C₄ alkyl group, for example, methyl, ethyl, propyl group, isopropyl group, butyl group, t-butyl, pentyl or the like, and the hydrogen of the alkyl group may be replaced by deuterium.

The aryl group, the arylene group, the fluorenyl group, the fluorenylene group, the heterocyclic group, the alicyclic group group, the fused ring, the alkyl group, the alkenyl group, the alkynyl group, the alkoxyl group, the aryloxyl group, and the ring formed by adjacent groups may be each substituted with one or more substituents selected from the group consisting of deuterium, halogen, a silane group unsubstituted or substituted with a C₁-C₂₀ alkyl group or a C₆-C₂₀ aryl group, a phosphine oxide substituted or unsubstituted with a C₁-C₂₀ alkyl group or a C₆-C₂₀ aryl group, a cyano group, a C₁-C₂₀ alkoxy group, a C₆-C₃₀ aryloxy group, a C₁-C₂₀ alkyl group, a C₂-C₂₀ alkenyl group, a C₂-C₂₀ alkynyl group, a C₆-C₃₀ aryl group, a fluorenyl group, a C₂-C₃₀ heterocyclic group comprising at least one heteroatom selected from the group consisting of O, N, S, Si and P, a C₃-C₃₀ alicyclic group, and a fused ring of a C₆-C₃₀ aromatic ring and a C₃-C₃₀ alicyclic group, the adjacent substituents may be bonded to each other to form a ring, and hydrogen of the substituents may be replaced with deuterium.

Preferably, at least one of the aryl group, the arylene group, the fluorenyl group, the fluorenylene group, the heterocyclic group, the alicyclic group, the fused ring, the alkyl group, the alkenyl group, the alkynyl group, the alkoxyl group, the aryloxyl group, and the ring formed by adjacent groups may be substituted with one or more substituents selected from the group consisting of a C₁-C₂₀ alkyl group, a C₆-C₃₀ aryl group, a fluorenyl group, a C₂-C₃₀ heterocyclic group comprising at least one heteroatom selected from the group consisting of O, N, S, Si and P, and a C₃-C₃₀ alicyclic group, and hydrogen of the substituents may be replaced with deuterium.

Formula A may be one of the following Formula A-1 to Formula A-4.

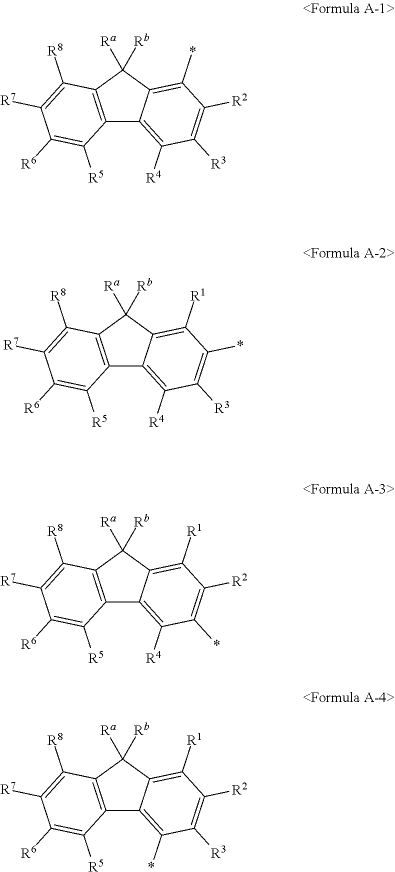

In Formula A-1 to Formula A-4, R¹ to R⁸, R^{a}, R^{b} are the same as defined in Formula A.

Formula B may be one of the following Formula B-1 to Formula B-4.

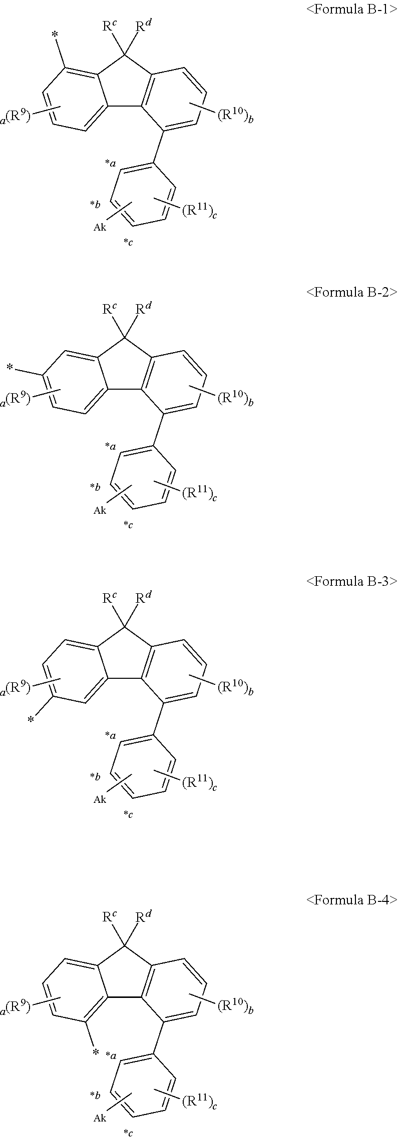

In Formula B-1 to Formula B-4, R⁹ to R¹¹, R^{c}, R^{d}, Ak, a to c are the same as defined in Formula B.

Ak may be selected from the group consisting of Formula Ak-1 to Formula Ak-8, but is not limited thereto.

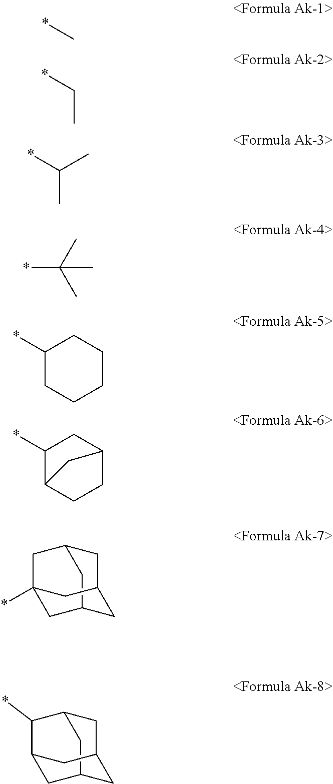

In Formula Ak-1 to Formula Ak-8, the asterisk (*) indicates the bonding position, and hydrogen may be replaced by deuterium.

Ar may be selected from the group consisting of Formula Ar-1 to Ar-11, but is not limited thereto.

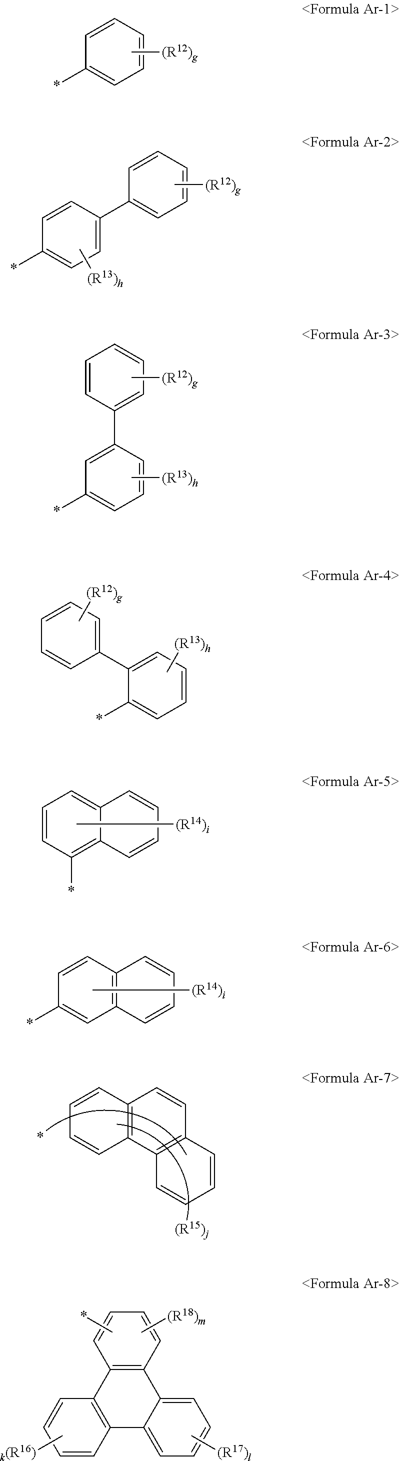

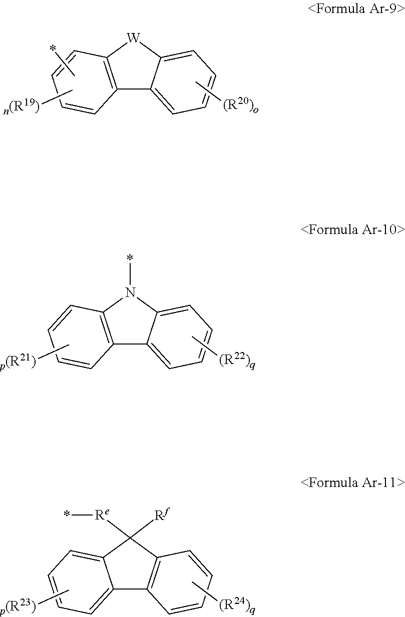

In Formula Ar-1 to Formula Ar-11, each symbol is defined as follows.

W is O, S, C(R²⁵)(R²⁶) or N(R²⁷).

R¹² to R²⁶, R^{f} are each independently selected from the group consisting of hydrogen, deuterium, halogen, a silane group unsubstituted or substituted with a C₁-C₂₀ alkyl group or a C₆-C₂₀ aryl group, a phosphine oxide substituted or unsubstituted with a C₁-C₂₀ alkyl group or a C₆-C₂₀ aryl group, a cyano group, a C₁-C₂₀ alkoxy group, a C₆-C₃₀ aryloxy group, a C₁-C₂₀ alkyl group, a C₂-C₂₀ alkenyl group, a C₂-C₂₀ alkynyl group, a C₆-C₃₀ aryl group, a fluorenyl group, a C₂-C₃₀ heterocyclic group comprising at least one heteroatom selected from the group consisting of O, N, S, Si and P, a C₃-C₃₀ alicyclic group, and a fused ring of a C₆-C₃₀ aromatic ring and a C₃-C₃₀ alicyclic group, the adjacent groups may combine with each other to form a ring, R²⁵ and R²⁶ may combine with each other to form a ring, and hydrogen of the substituents may be replaced with deuterium. When R²⁵ and R²⁶ may combine with each other to form a ring, a spiro compound may be formed.

R^{e} is selected from the group consisting of a single bond, a C₁-C₂₀ alkylene group, a C₆-C₃₀ arylene group, a fluorenylene group, a C₂-C₃₀ heterocyclic group comprising at least one heteroatom selected from the group consisting of O, N, S, Si and P, a C₃-C₃₀ alicyclic group, and a fused ring of a C₆-C₃₀ aromatic ring and a C₃-C₃₀ alicyclic group, R^{e} and R^{f} may combine with each other to form a ring. When R^{e} and R^{f} may combine with each other to form a ring, a spiro compound may be formed.

R²⁷ is selected from the group consisting of a C₁-C₂₀ alkyl group, a C₆-C₃₀ aryl group, a fluorenyl group, a C₂-C₃₀ heterocyclic group comprising at least one heteroatom selected from the group consisting of O, N, S, Si and P, a C₃-C₃₀ alicyclic group, and a fused ring of a C₆-C₃₀ aromatic ring and a C₃-C₃₀ alicyclic group.

g is an integer from 0 to 5; h, k, l, o, p and q are each an integer from 0 to 4; i is an integer from 0 to 7; j is an integer from 0 to 9; and n and m are each an integer from 0 to 3. When these are an integer of 2 or more, each of the plurality of R¹² to each of the plurality of R²⁴ are the same as or different from each other.

In Formula 1, at least one of L¹ to L³ may be a single bond or may be selected from the group consisting of Formula L-1 to Formula L-27, but is not limited thereto.

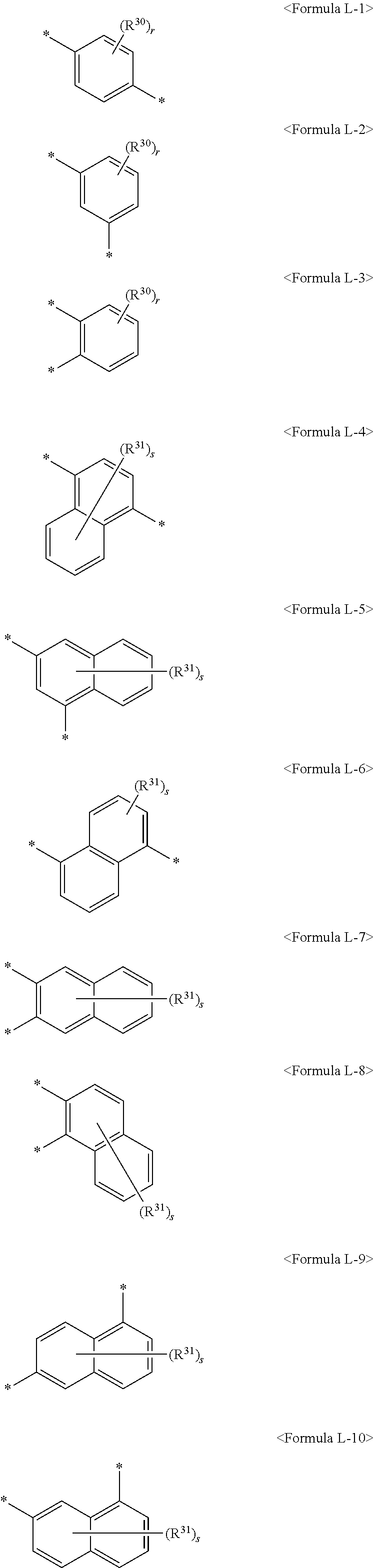

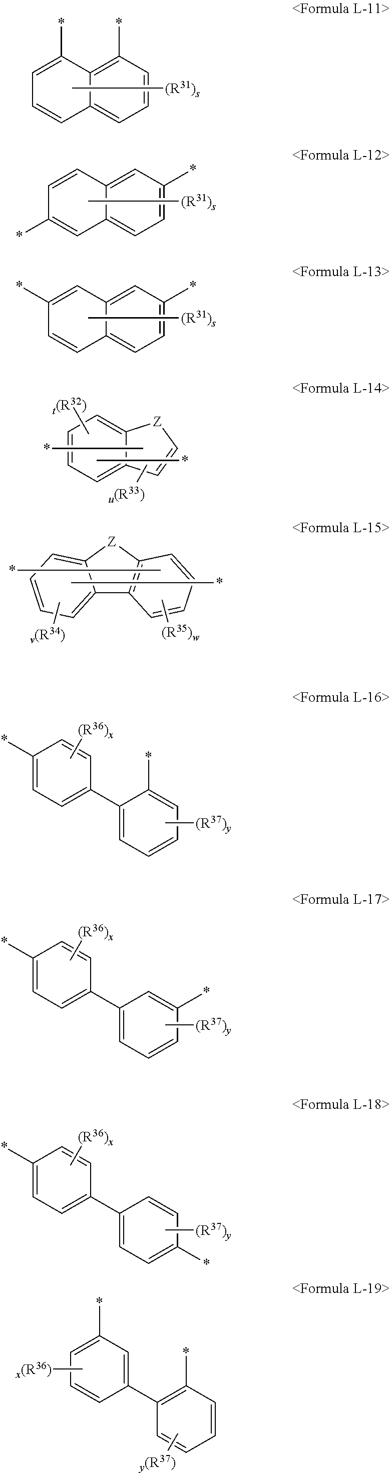

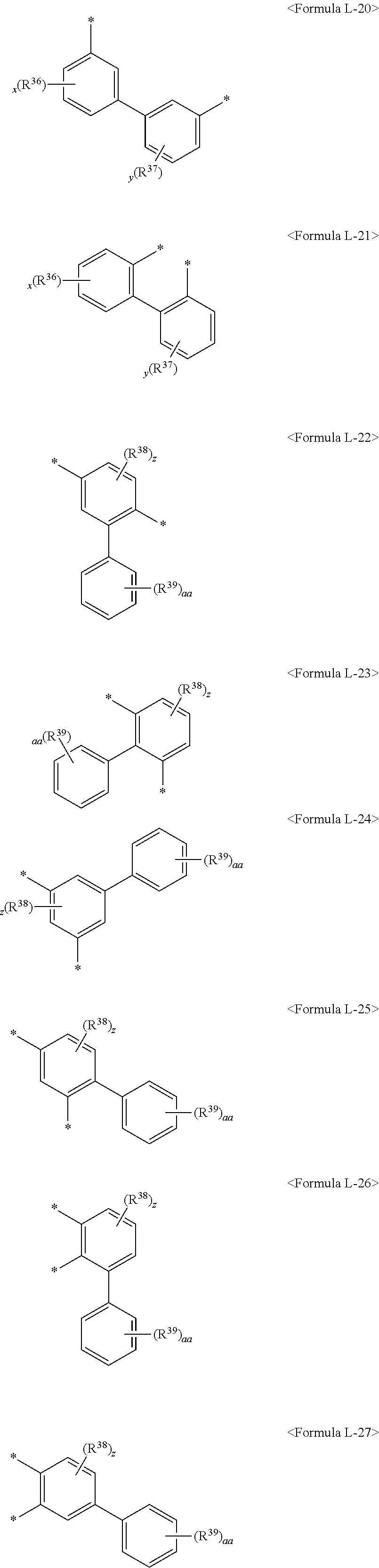

In Formula L-1 to Formula L-27, each symbol is defined as follows.

Z is O, S, C(R⁴⁰), C(R⁴⁰)(R⁴¹), N or N(R⁴²), with the proviso that Z is C(R⁴⁰) or N when connected through Z.

R³⁰ to R³⁹, R⁴⁰, R⁴¹ are each independently selected from the group consisting of hydrogen, deuterium, halogen, a silane group unsubstituted or substituted with a C₁-C₂₀ alkyl group or a C₆-C₂₀ aryl group, a phosphine oxide substituted or unsubstituted with a C₁-C₂₀ alkyl group or a C₆-C₂₀ aryl group, a cyano group, a nitro group, a C₁-C₂₀ alkylthio group, a C₁-C₂₀ alkoxy group, a C₆-C₃₀ aryloxy group, a C₆-C₃₀₂| arylthio, a C₁-C₂₀ alkyl group, a C₂-C₂₀ alkenyl group, a C₂-C₂₀ alkynyl group, a C₆-C₃₀ aryl group, a C₆-C₃₀ aryl group substituted with deuterium, a C₃-C₃₀ alicyclic group, a fused ring of a C₆-C₃₀ aromatic ring and a C₃-C₃₀ alicyclic group, and a C₂-C₃₀ heterocyclic group comprising at least one heteroatom selected from the group consisting of O, N, S, Si and P, and the adjacent groups may combine with each other to form a ring.

R⁴² is selected from the group consisting of a C₁-C₂₀ alkyl group, a C₆-C₃₀ aryl group, a C₆-C₃₀ aryl group substituted with deuterium, a C₃-C₃₀ alicyclic group, a fused ring of a C₆-C₃₀ aromatic ring and a C₃-C₃₀ alicyclic group, and a C₂-C₃₀ heterocyclic group comprising at least one heteroatom selected from the group consisting of O, N, S, Si and P.

r, t, v, w, x and y are each an integer from 0 to 4; s is an integer from 0 to 6; u is an integer from 0 to 2; z is an integer from 0 to 4; and aa is an integer from 0 to 5. When these are each an integer of 2 or more, each of the plurality of R³⁰ to each of the plurality of R³⁹ are the same as or different from each other.

Specifically, compound represented by Formula 1 may be one of the following compounds, but is not limited thereto.

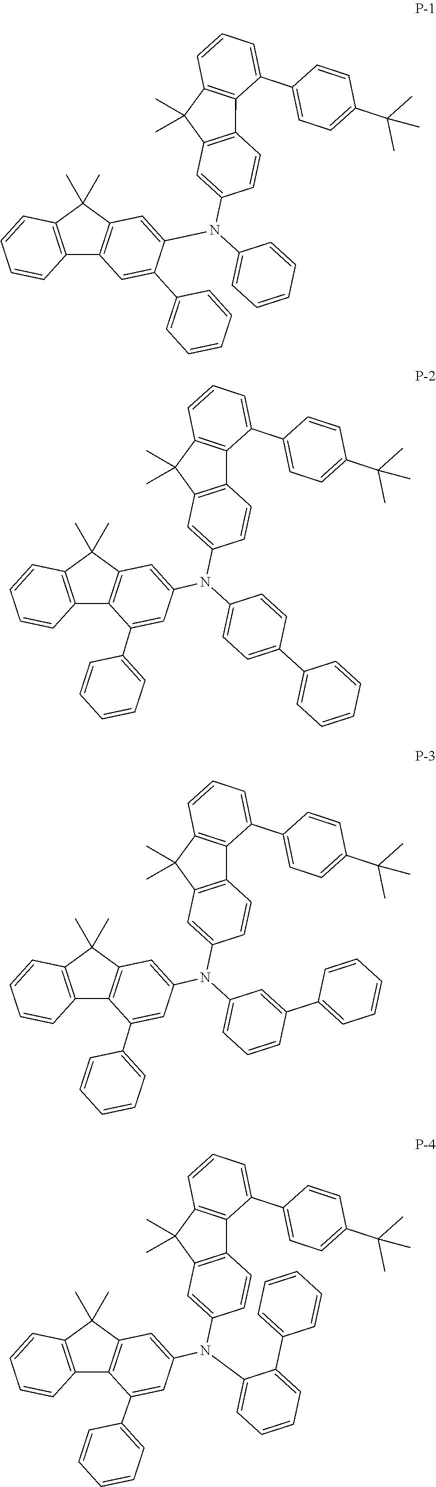

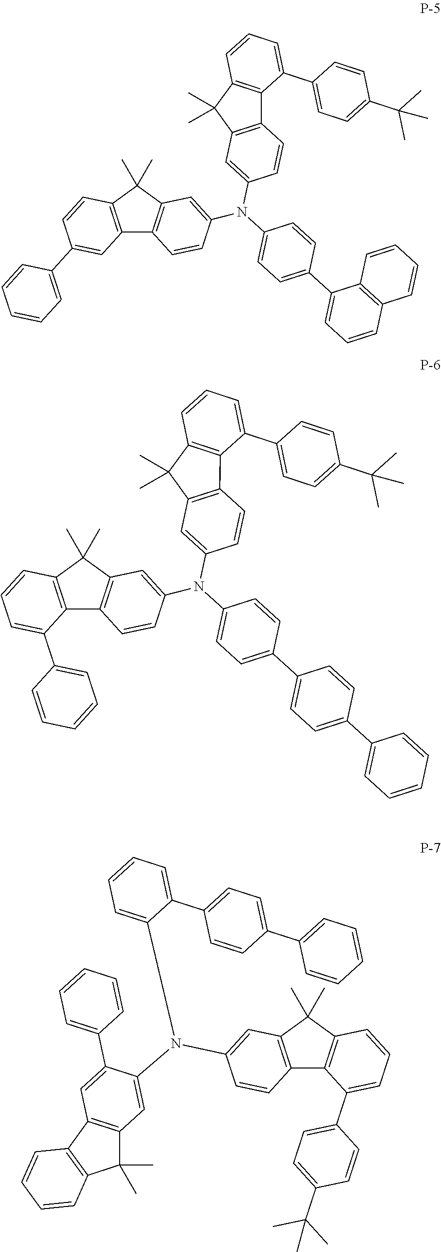 [SMILES: CC(C)(C)c1ccc(-c2cccc3c2-c2ccc(N(c4ccc(-c5cccc6ccccc56)cc4)c4ccc5c(c4)C(C)(C)c4ccc(-c6ccccc6)cc4-5)cc2C3(C)C)cc1 ; CC(C)(C)c1ccc(-c2cccc3c2-c2ccc(N(c4ccc(-c5ccc(-c6ccccc6)cc5)cc4)c4ccc5c(c4)C(C)(C)c4cccc(-c6ccccc6)c4-5)cc2C3(C)C)cc1 ; CC(C)(C)c1ccc(-c2cccc3c2-c2ccc(N(c4ccccc4-c4ccc(-c5ccccc5)cc4)c4cc5c(cc4-c4ccccc4)-c4ccccc4C5(C)C)cc2C3(C)C)cc1]

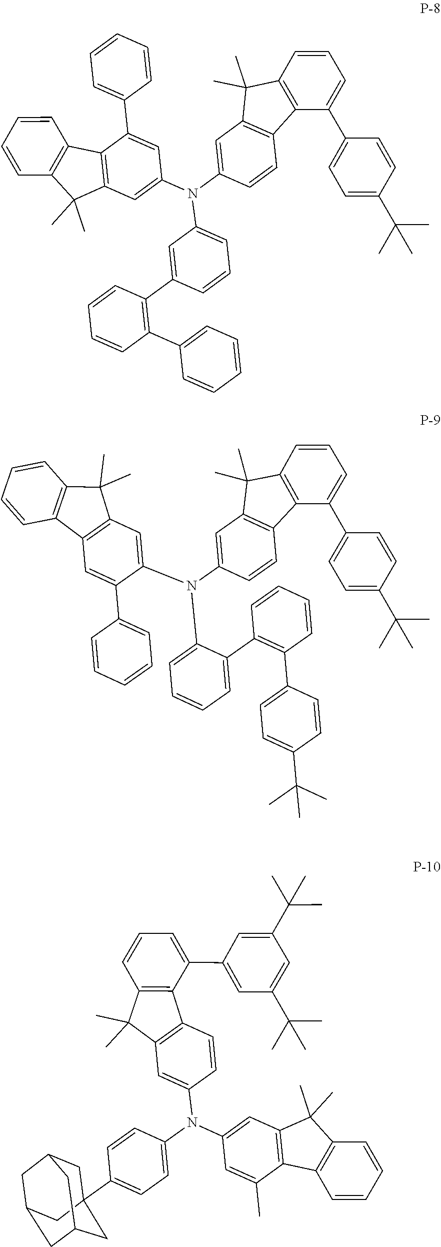 [SMILES: CC(C)(C)c1ccc(-c2cccc3c2-c2ccc(N(c4cccc(-c5ccccc5-c5ccccc5)c4)c4cc(-c5ccccc5)c5c(c4)C(C)(C)c4ccccc4-5)cc2C3(C)C)cc1 ; CC(C)(C)c1ccc(-c2ccccc2-c2ccccc2N(c2ccc3c(c2)C(C)(C)c2cccc(-c4ccc(C(C)(C)C)cc4)c2-3)c2cc3c(cc2-c2ccccc2)-c2ccccc2C3(C)C)cc1 ; Cc1cc(N(c2ccc(C34CC5CC(CC(C5)C3)C4)cc2)c2ccc3c(c2)C(C)(C)c2cccc(-c4cc(C(C)(C)C)cc(C(C)(C)C)c4)c2-3)cc2c1-c1ccccc1C2(C)C]

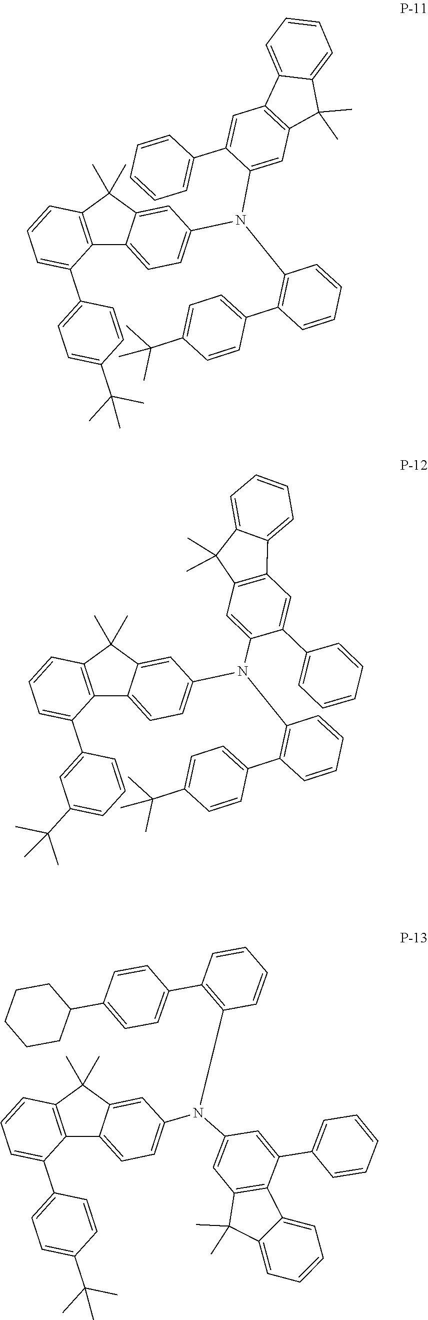

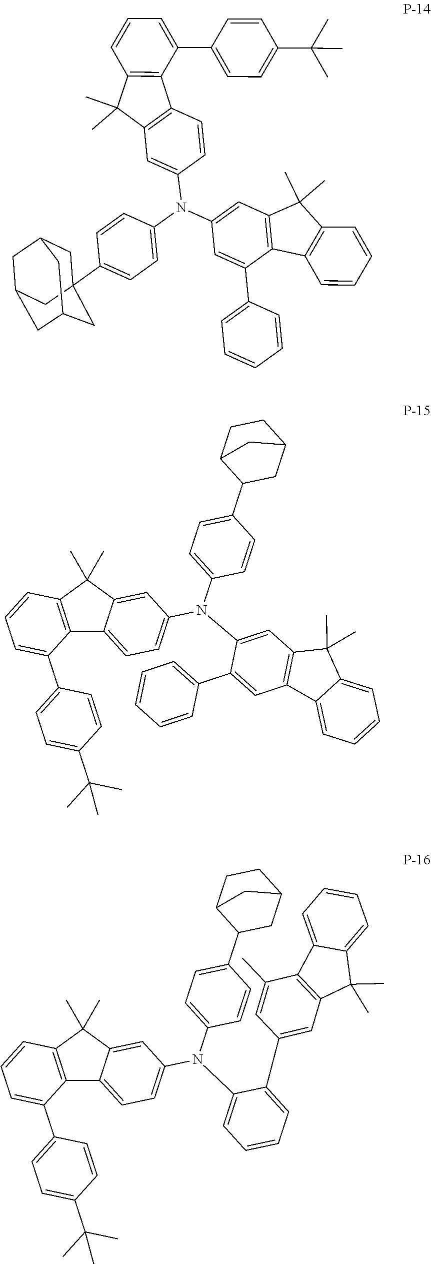 [SMILES: CC(C)(C)c1ccc(-c2cccc3c2-c2ccc(N(c4ccc(C56CC7CC(CC(C7)C5)C6)cc4)c4cc(-c5ccccc5)c5c(c4)C(C)(C)c4ccccc4-5)cc2C3(C)C)cc1 ; CC(C)(C)c1ccc(-c2cccc3c2-c2ccc(N(c4ccc(C5CC6CCC5C6)cc4)c4cc5c(cc4-c4ccccc4)-c4ccccc4C5(C)C)cc2C3(C)C)cc1 ; Cc1cc(-c2ccccc2N(c2ccc(C3CC4CCC3C4)cc2)c2ccc3c(c2)C(C)(C)c2cccc(-c4ccc(C(C)(C)C)cc4)c2-3)cc2c1-c1ccccc1C2(C)C]

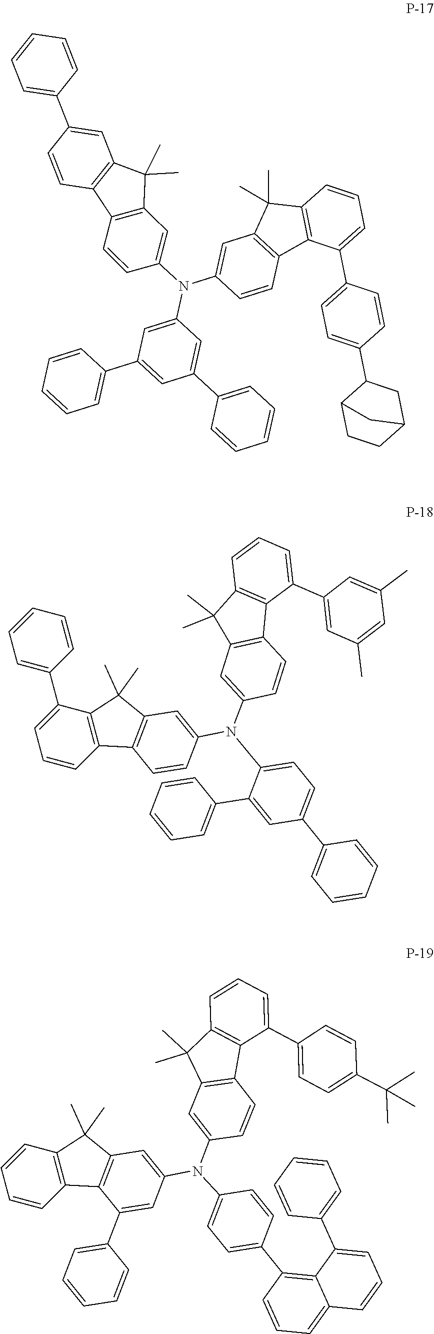 [SMILES: CC1(C)c2cc(-c3ccccc3)ccc2-c2ccc(N(c3cc(-c4ccccc4)cc(-c4ccccc4)c3)c3ccc4c(c3)C(C)(C)c3cccc(-c5ccc(C6CC7CCC6C7)cc5)c3-4)cc21 ; Cc1cc(C)cc(-c2cccc3c2-c2ccc(N(c4ccc5c(c4)C(C)(C)c4c(-c6ccccc6)cccc4-5)c4ccc(-c5ccccc5)cc4-c4ccccc4)cc2C3(C)C)c1 ; CC(C)(C)c1ccc(-c2cccc3c2-c2ccc(N(c4ccc(-c5cccc6cccc(-c7ccccc7)c56)cc4)c4cc(-c5ccccc5)c5c(c4)C(C)(C)c4ccccc4-5)cc2C3(C)C)cc1]

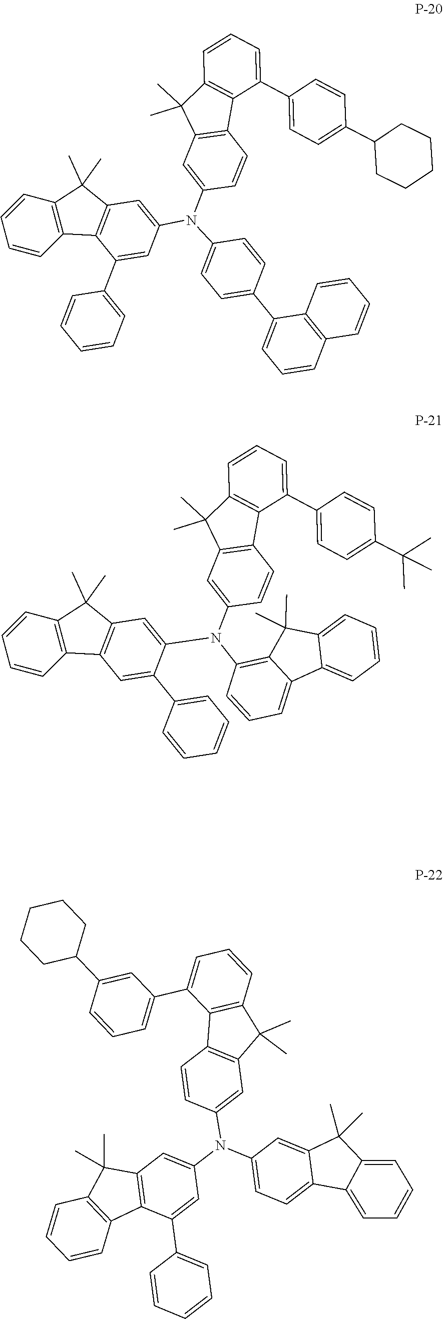 [SMILES: CC1(C)c2cc(N(c3ccc(-c4cccc5ccccc45)cc3)c3cc(-c4ccccc4)c4c(c3)C(C)(C)c3ccccc3-4)ccc2-c2c(-c3ccc(C4CCCCC4)cc3)cccc21 ; CC(C)(C)c1ccc(-c2cccc3c2-c2ccc(N(c4cc5c(cc4-c4ccccc4)-c4ccccc4C5(C)C)c4cccc5c4C(C)(C)c4ccccc4-5)cc2C3(C)C)cc1 ; CC1(C)c2ccccc2-c2ccc(N(c3ccc4c(c3)C(C)(C)c3cccc(-c5cccc(C6CCCCC6)c5)c3-4)c3cc(-c4ccccc4)c4c(c3)C(C)(C)c3ccccc3-4)cc21]

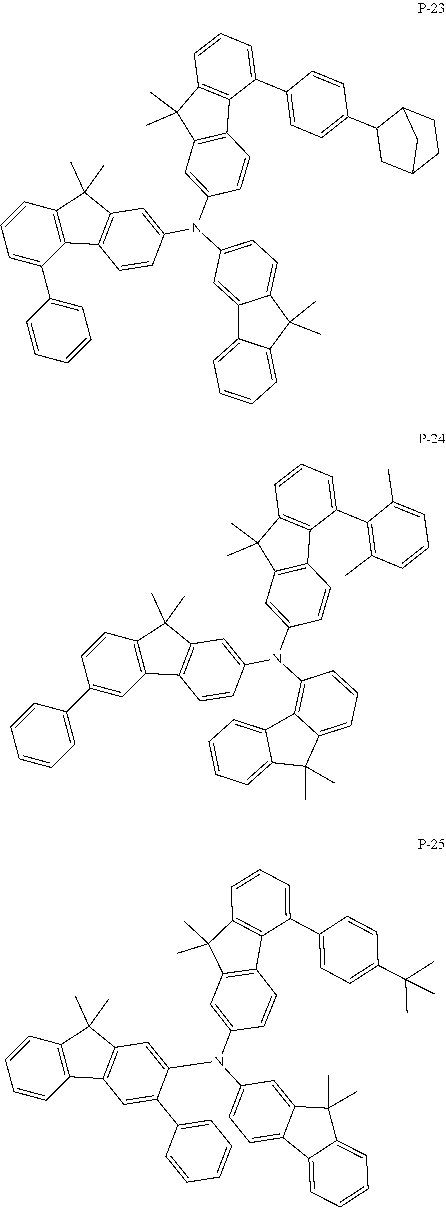 [SMILES: CC1(C)c2ccccc2-c2cc(N(c3ccc4c(c3)C(C)(C)c3cccc(-c5ccccc5)c3-4)c3ccc4c(c3)C(C)(C)c3cccc(-c5ccc(C6CC7CCC6C7)cc5)c3-4)ccc21 ; Cc1cccc(C)c1-c1cccc2c1-c1ccc(N(c3ccc4c(c3)C(C)(C)c3ccc(-c5ccccc5)cc3-4)c3cccc4c3-c3ccccc3C4(C)C)cc1C2(C)C ; CC(C)(C)c1ccc(-c2cccc3c2-c2ccc(N(c4ccc5c(c4)C(C)(C)c4ccccc4-5)c4cc5c(cc4-c4ccccc4)-c4ccccc4C5(C)C)cc2C3(C)C)cc1]

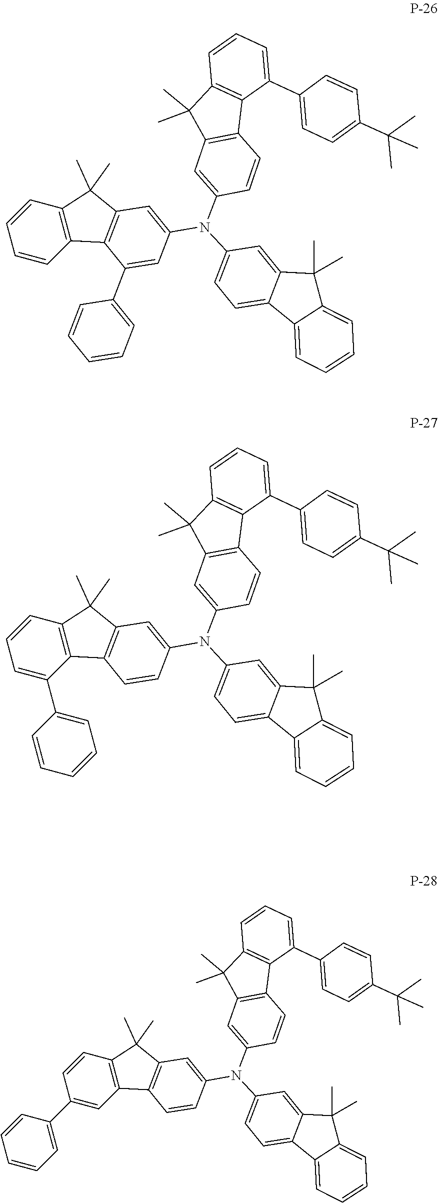 [SMILES: CC(C)(C)c1ccc(-c2cccc3c2-c2ccc(N(c4ccc5c(c4)C(C)(C)c4ccccc4-5)c4cc(-c5ccccc5)c5c(c4)C(C)(C)c4ccccc4-5)cc2C3(C)C)cc1 ; CC(C)(C)c1ccc(-c2cccc3c2-c2ccc(N(c4ccc5c(c4)C(C)(C)c4ccccc4-5)c4ccc5c(c4)C(C)(C)c4cccc(-c6ccccc6)c4-5)cc2C3(C)C)cc1 ; CC(C)(C)c1ccc(-c2cccc3c2-c2ccc(N(c4ccc5c(c4)C(C)(C)c4ccccc4-5)c4ccc5c(c4)C(C)(C)c4ccc(-c6ccccc6)cc4-5)cc2C3(C)C)cc1]

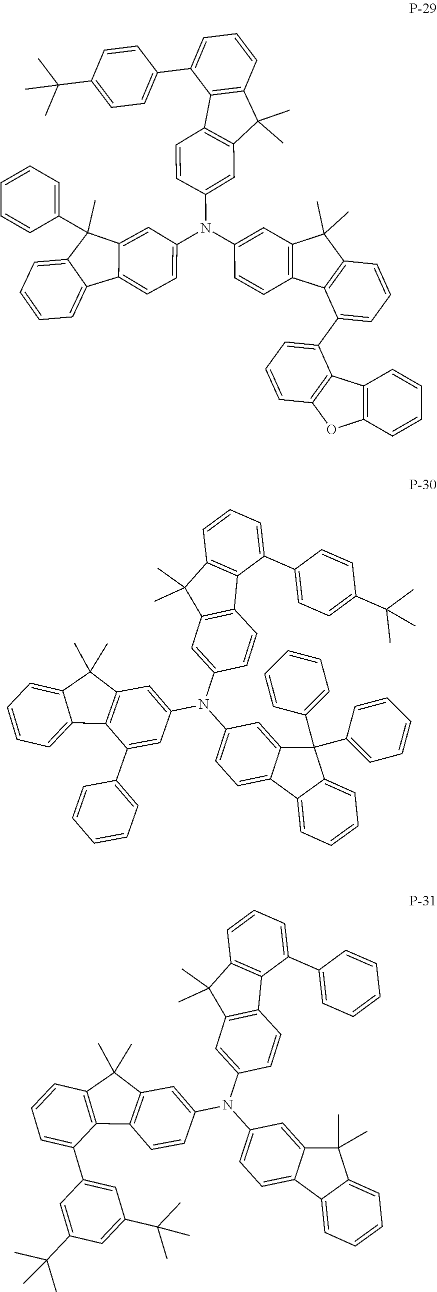

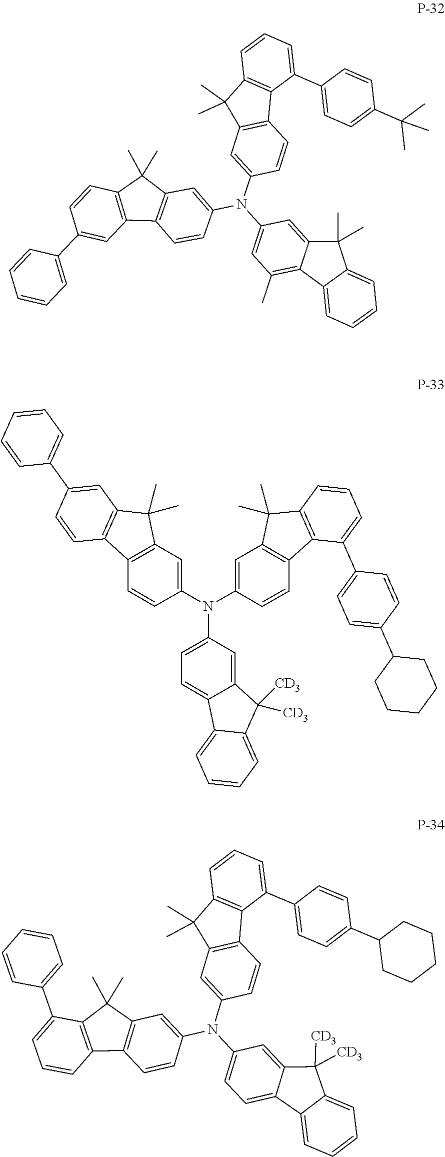 [SMILES: Cc1cc(N(c2ccc3c(c2)C(C)(C)c2ccc(-c4ccccc4)cc2-3)c2ccc3c(c2)C(C)(C)c2cccc(-c4ccc(C(C)(C)C)cc4)c2-3)cc2c1-c1ccccc1C2(C)C ; [2H]C([2H])([2H])C1(C([2H])([2H])[2H])c2ccccc2-c2ccc(N(c3ccc4c(c3)C(C)(C)c3cc(-c5ccccc5)ccc3-4)c3ccc4c(c3)C(C)(C)c3cccc(-c5ccc(C6CCCCC6)cc5)c3-4)cc21 ; [2H]C([2H])([2H])C1(C([2H])([2H])[2H])c2ccccc2-c2ccc(N(c3ccc4c(c3)C(C)(C)c3cccc(-c5ccc(C6CCCCC6)cc5)c3-4)c3ccc4c(c3)C(C)(C)c3c(-c5ccccc5)cccc3-4)cc21]

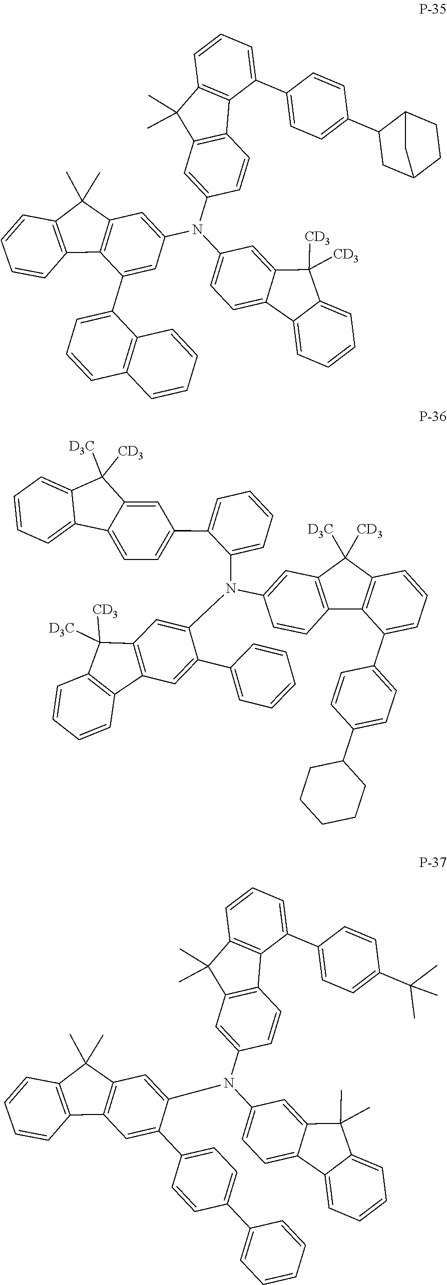

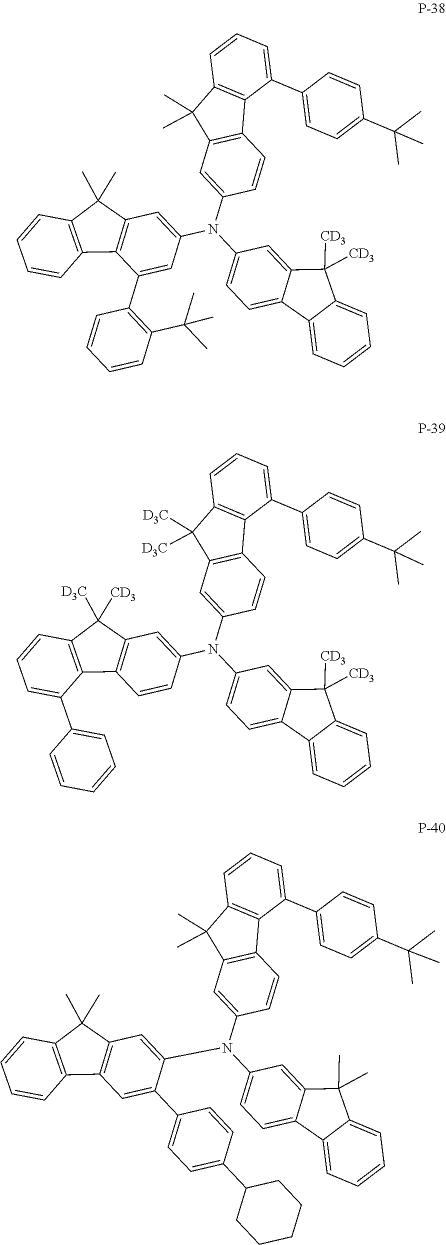 [SMILES: [2H]C([2H])([2H])C1(C([2H])([2H])[2H])c2ccccc2-c2ccc(N(c3ccc4c(c3)C(C)(C)c3cccc(-c5ccc(C(C)(C)C)cc5)c3-4)c3cc(-c4ccccc4C(C)(C)C)c4c(c3)C(C)(C)c3ccccc3-4)cc21 ; [2H]C([2H])([2H])C1(C([2H])([2H])[2H])c2ccccc2-c2ccc(N(c3ccc4c(c3)C(C([2H])([2H])[2H])(C([2H])([2H])[2H])c3cccc(-c5ccccc5)c3-4)c3ccc4c(c3)C(C([2H])([2H])[2H])(C([2H])([2H])[2H])c3cccc(-c5ccc(C(C)(C)C)cc5)c3-4)cc21 ; CC(C)(C)c1ccc(-c2cccc3c2-c2ccc(N(c4ccc5c(c4)C(C)(C)c4ccccc4-5)c4cc5c(cc4-c4ccc(C6CCCCC6)cc4)-c4ccccc4C5(C)C)cc2C3(C)C)cc1]

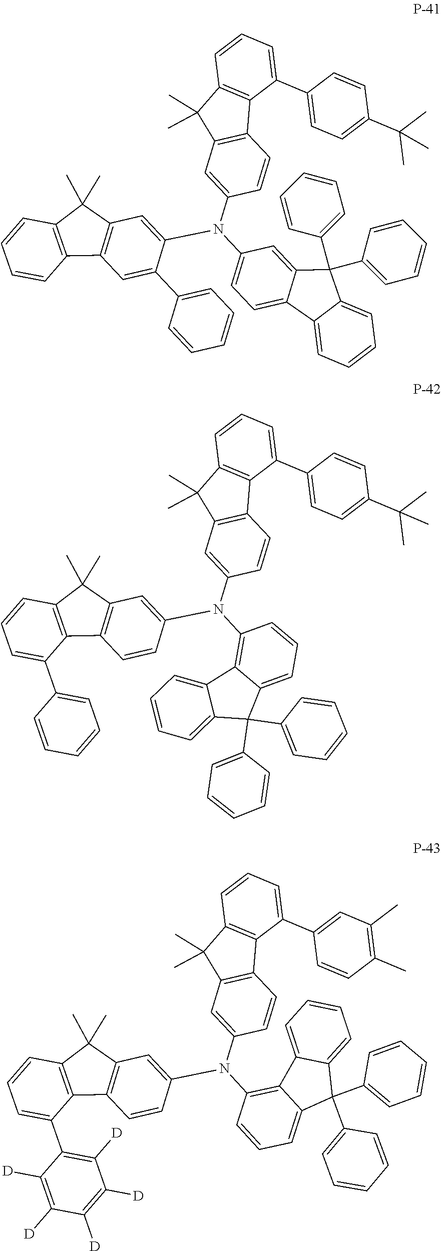 [SMILES: CC(C)(C)c1ccc(-c2cccc3c2-c2ccc(N(c4ccc5c(c4)C(c4ccccc4)(c4ccccc4)c4ccccc4-5)c4cc5c(cc4-c4ccccc4)-c4ccccc4C5(C)C)cc2C3(C)C)cc1 ; CC(C)(C)c1ccc(-c2cccc3c2-c2ccc(N(c4ccc5c(c4)C(C)(C)c4cccc(-c6ccccc6)c4-5)c4cccc5c4-c4ccccc4C5(c4ccccc4)c4ccccc4)cc2C3(C)C)cc1 ; [2H]c1c([2H])c([2H])c(-c2cccc3c2-c2ccc(N(c4ccc5c(c4)C(C)(C)c4cccc(-c6ccc(C)c(C)c6)c4-5)c4cccc5c4-c4ccccc4C5(c4ccccc4)c4ccccc4)cc2C3(C)C)c([2H])c1[2H]]

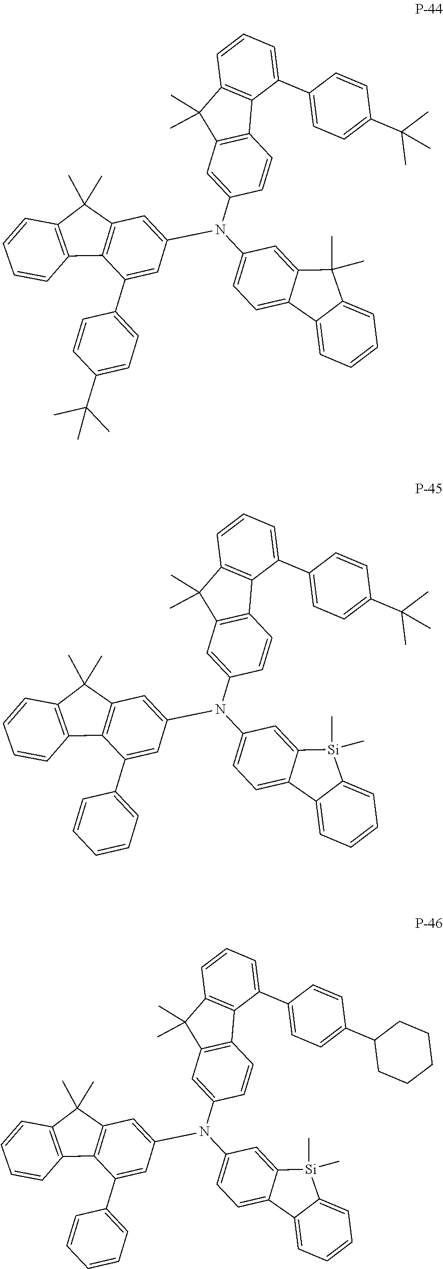 [SMILES: CC(C)(C)c1ccc(-c2cccc3c2-c2ccc(N(c4ccc5c(c4)C(C)(C)c4ccccc4-5)c4cc(-c5ccc(C(C)(C)C)cc5)c5c(c4)C(C)(C)c4ccccc4-5)cc2C3(C)C)cc1 ; CC(C)(C)c1ccc(-c2cccc3c2-c2ccc(N(c4cc(-c5ccccc5)c5c(c4)C(C)(C)c4ccccc4-5)c4ccc5c(c4)[Si](C)(C)c4ccccc4-5)cc2C3(C)C)cc1 ; CC1(C)c2cc(N(c3cc(-c4ccccc4)c4c(c3)C(C)(C)c3ccccc3-4)c3ccc4c(c3)[Si](C)(C)c3ccccc3-4)ccc2-c2c(-c3ccc(C4CCCCC4)cc3)cccc21]

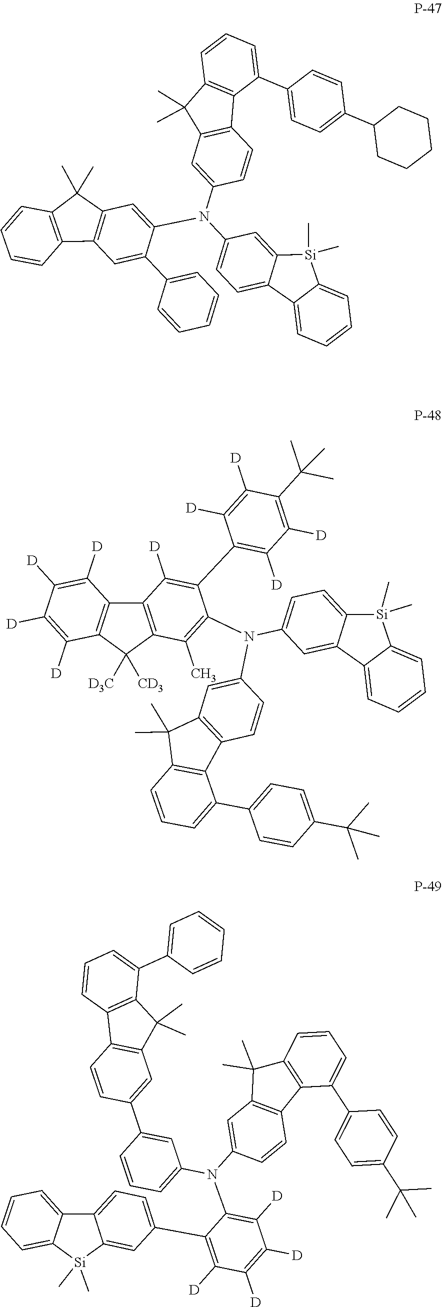

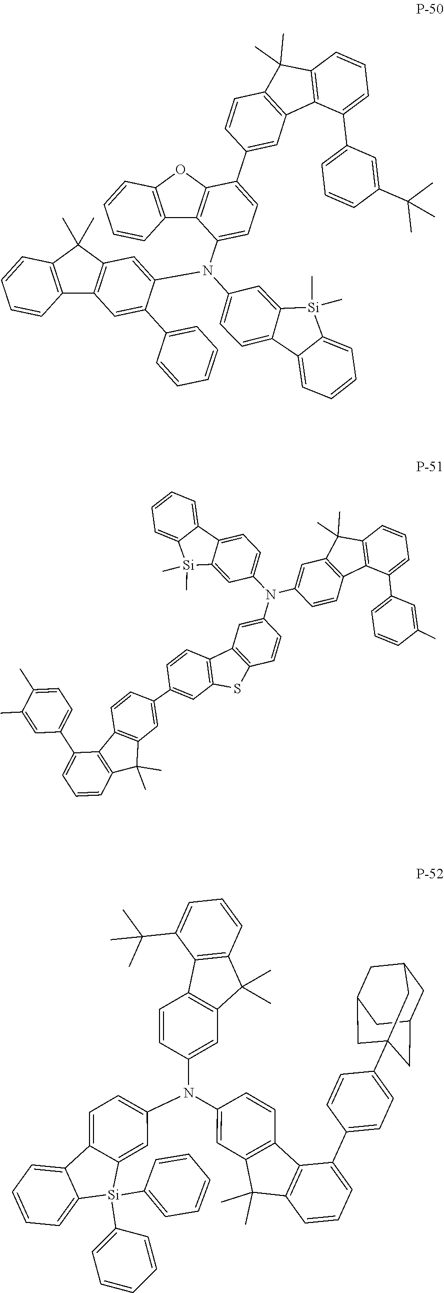

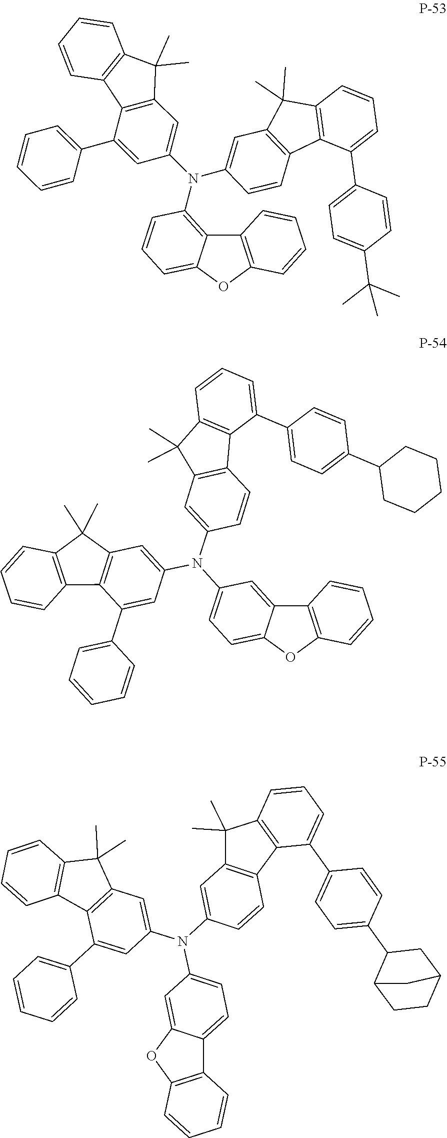 [SMILES: CC(C)(C)c1ccc(-c2cccc3c2-c2ccc(N(c4cc(-c5ccccc5)c5c(c4)C(C)(C)c4ccccc4-5)c4cccc5oc6ccccc6c45)cc2C3(C)C)cc1 ; CC1(C)c2cc(N(c3cc(-c4ccccc4)c4c(c3)C(C)(C)c3ccccc3-4)c3ccc4oc5ccccc5c4c3)ccc2-c2c(-c3ccc(C4CCCCC4)cc3)cccc21 ; CC1(C)c2cc(N(c3cc(-c4ccccc4)c4c(c3)C(C)(C)c3ccccc3-4)c3ccc4c(c3)oc3ccccc34)ccc2-c2c(-c3ccc(C4CC5CCC4C5)cc3)cccc21]

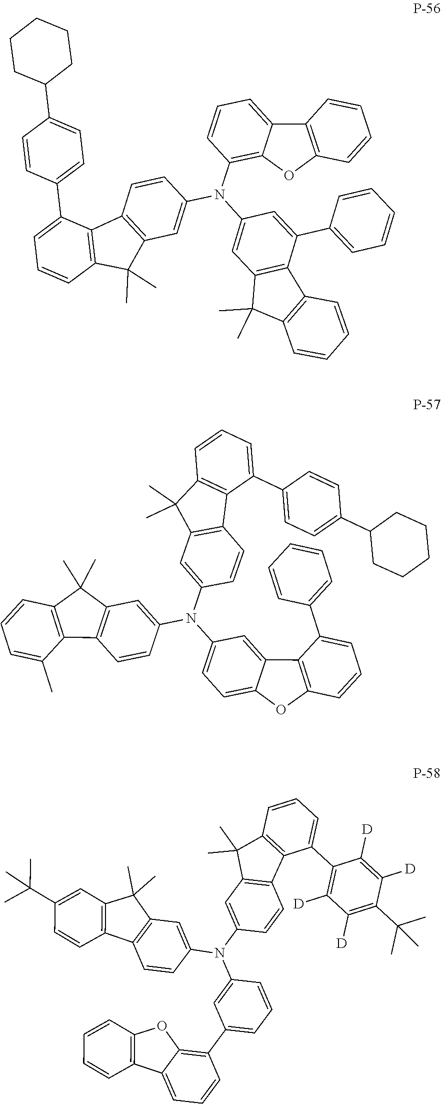 [SMILES: CC1(C)c2cc(N(c3cc(-c4ccccc4)c4c(c3)C(C)(C)c3ccccc3-4)c3cccc4c3oc3ccccc34)ccc2-c2c(-c3ccc(C4CCCCC4)cc3)cccc21 ; Cc1cccc2c1-c1ccc(N(c3ccc4c(c3)C(C)(C)c3cccc(-c5ccc(C6CCCCC6)cc5)c3-4)c3ccc4oc5cccc(-c6ccccc6)c5c4c3)cc1C2(C)C ; [2H]c1c([2H])c(C(C)(C)C)c([2H])c([2H])c1-c1cccc2c1-c1ccc(N(c3cccc(-c4cccc5c4oc4ccccc45)c3)c3ccc4c(c3)C(C)(C)c3cc(C(C)(C)C)ccc3-4)cc1C2(C)C]

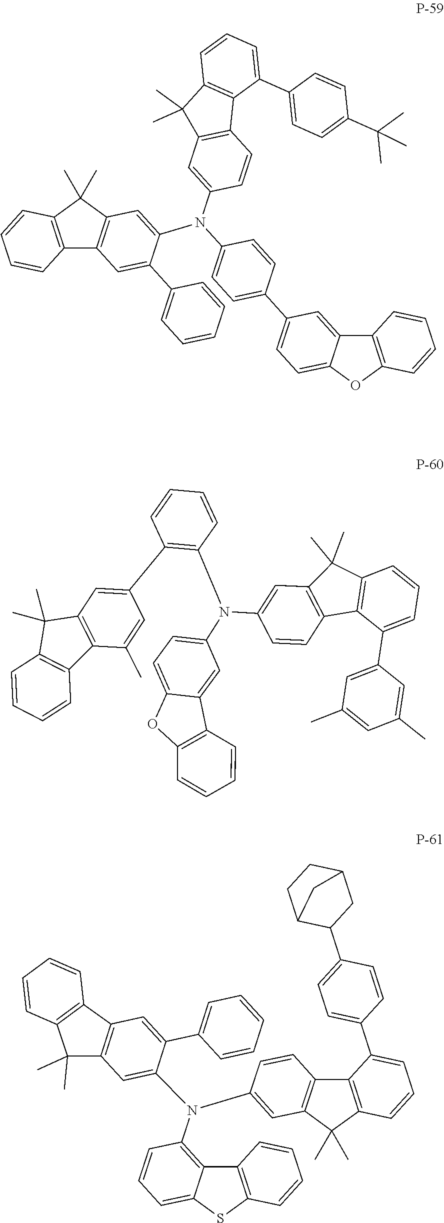 [SMILES: CC(C)(C)c1ccc(-c2cccc3c2-c2ccc(N(c4ccc(-c5ccc6oc7ccccc7c6c5)cc4)c4cc5c(cc4-c4ccccc4)-c4ccccc4C5(C)C)cc2C3(C)C)cc1 ; Cc1cc(C)cc(-c2cccc3c2-c2ccc(N(c4ccc5oc6ccccc6c5c4)c4ccccc4-c4cc(C)c5c(c4)C(C)(C)c4ccccc4-5)cc2C3(C)C)c1 ; CC1(C)c2ccccc2-c2cc(-c3ccccc3)c(N(c3ccc4c(c3)C(C)(C)c3cccc(-c5ccc(C6CC7CCC6C7)cc5)c3-4)c3cccc4sc5ccccc5c34)cc21]

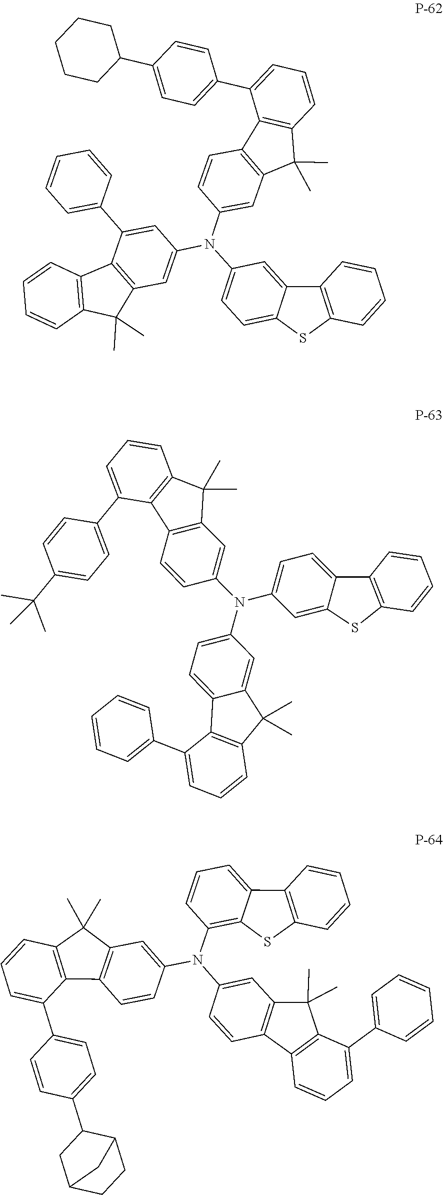 [SMILES: CC1(C)c2cc(N(c3cc(-c4ccccc4)c4c(c3)C(C)(C)c3ccccc3-4)c3ccc4sc5ccccc5c4c3)ccc2-c2c(-c3ccc(C4CCCCC4)cc3)cccc21 ; CC(C)(C)c1ccc(-c2cccc3c2-c2ccc(N(c4ccc5c(c4)C(C)(C)c4cccc(-c6ccccc6)c4-5)c4ccc5c(c4)sc4ccccc45)cc2C3(C)C)cc1 ; CC1(C)c2cc(N(c3ccc4c(c3)C(C)(C)c3c(-c5ccccc5)cccc3-4)c3cccc4c3sc3ccccc34)ccc2-c2c(-c3ccc(C4CC5CCC4C5)cc3)cccc21]

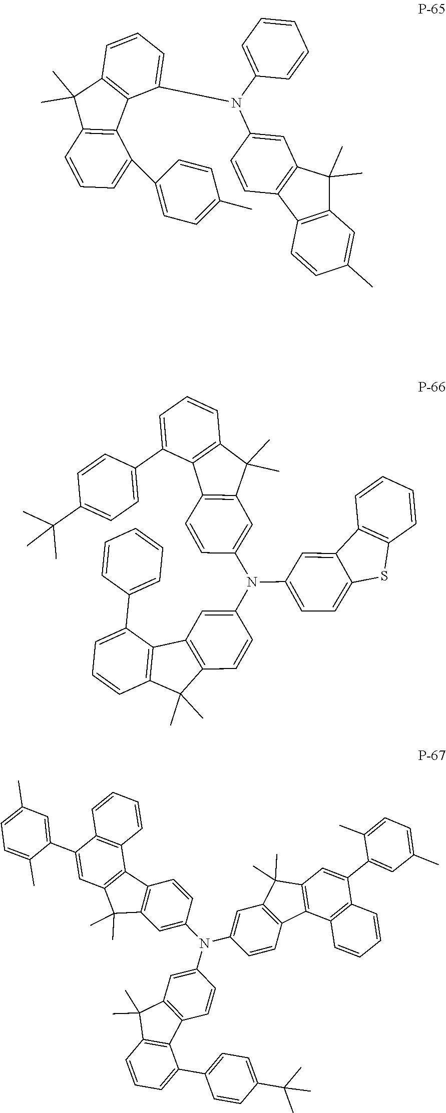

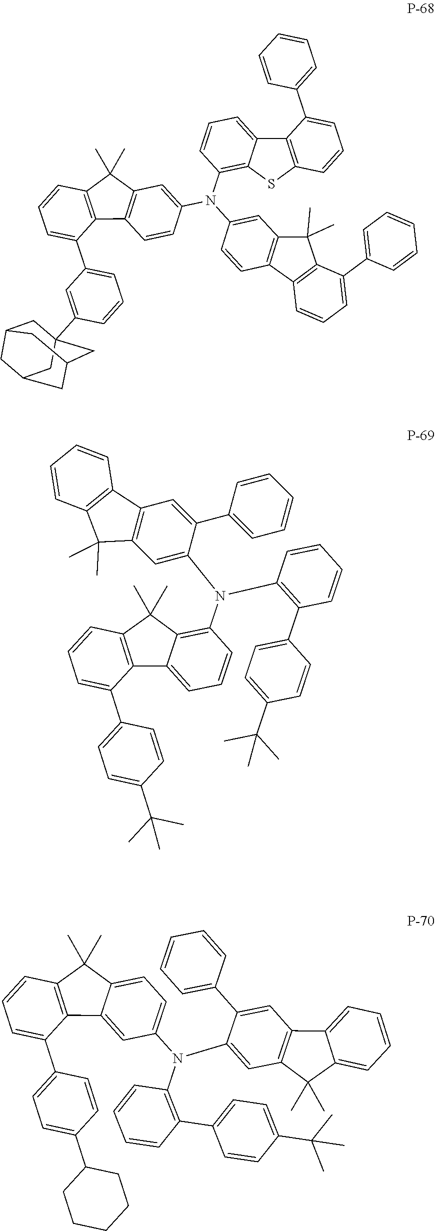 [SMILES: CC1(C)c2cc(N(c3ccc4c(c3)C(C)(C)c3c(-c5ccccc5)cccc3-4)c3cccc4c3sc3cccc(-c5ccccc5)c34)ccc2-c2c(-c3cccc(C45CC6CC(CC(C6)C4)C5)c3)cccc21 ; CC(C)(C)c1ccc(-c2ccccc2N(c2cc3c(cc2-c2ccccc2)-c2ccccc2C3(C)C)c2cccc3c2C(C)(C)c2cccc(-c4ccc(C(C)(C)C)cc4)c2-3)cc1 ; CC(C)(C)c1ccc(-c2ccccc2N(c2ccc3c(c2)-c2c(-c4ccc(C5CCCCC5)cc4)cccc2C3(C)C)c2cc3c(cc2-c2ccccc2)-c2ccccc2C3(C)C)cc1]

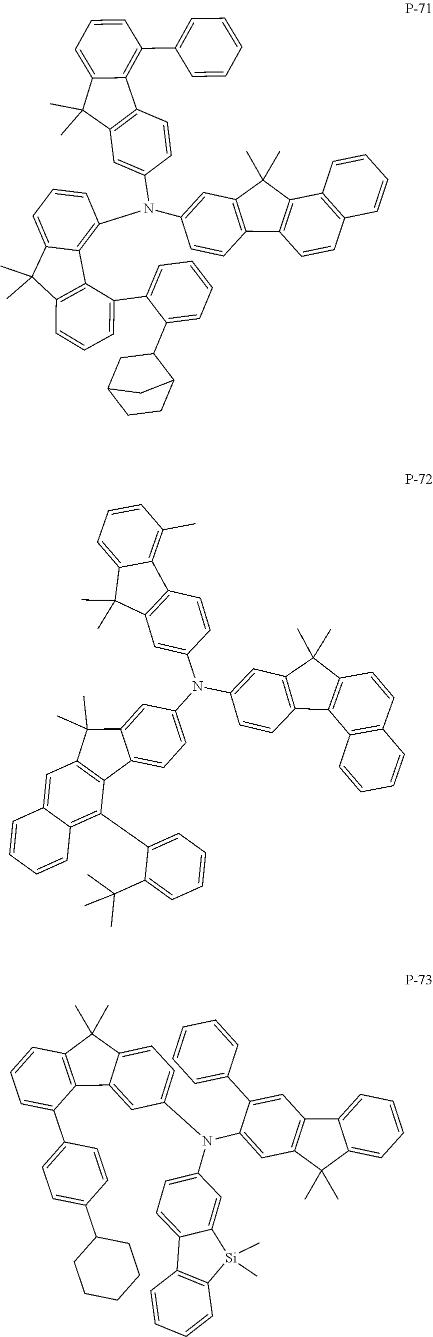 [SMILES: CC1(C)c2cc(N(c3ccc4c(c3)C(C)(C)c3c-4ccc4ccccc34)c3cccc4c3-c3c(-c5ccccc5C5CC6CCC5C6)cccc3C4(C)C)ccc2-c2c(-c3ccccc3)cccc21 ; Cc1cccc2c1-c1ccc(N(c3ccc4c(c3)C(C)(C)c3cc5ccccc5c(-c5ccccc5C(C)(C)C)c3-4)c3ccc4c(c3)C(C)(C)c3ccc5ccccc5c3-4)cc1C2(C)C ; CC1(C)c2ccccc2-c2cc(-c3ccccc3)c(N(c3ccc4c(c3)-c3c(-c5ccc(C6CCCCC6)cc5)cccc3C4(C)C)c3ccc4c(c3)[Si](C)(C)c3ccccc3-4)cc21]

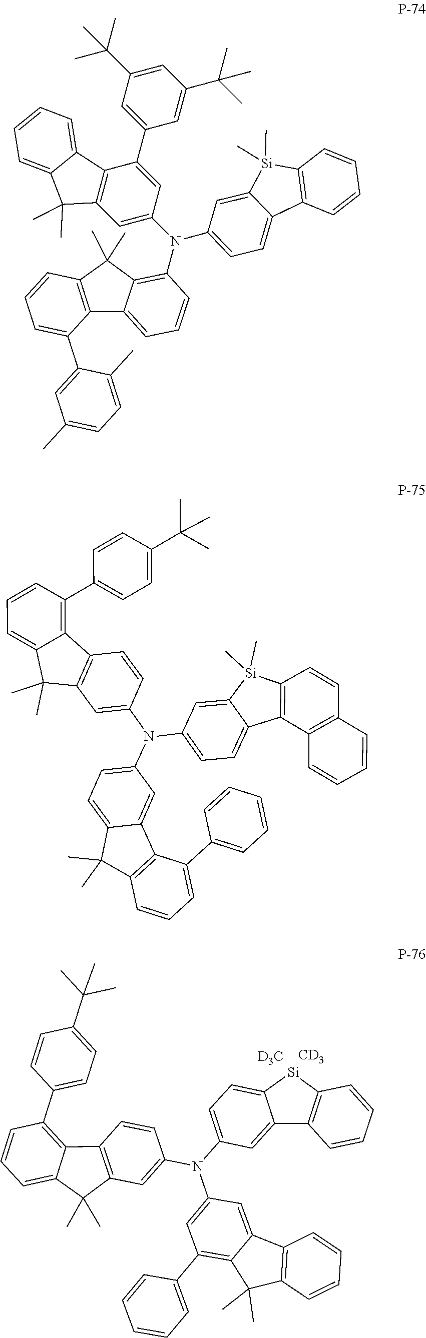

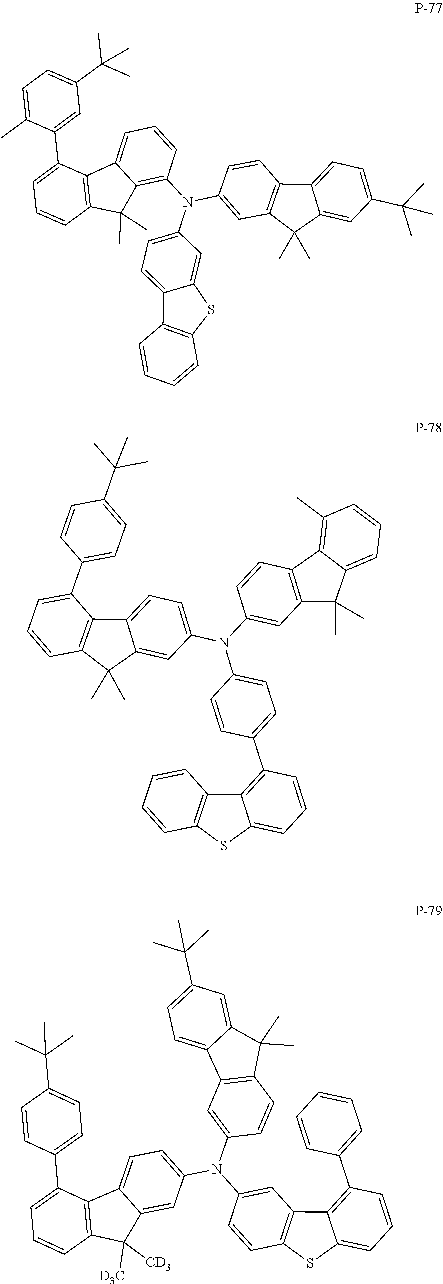

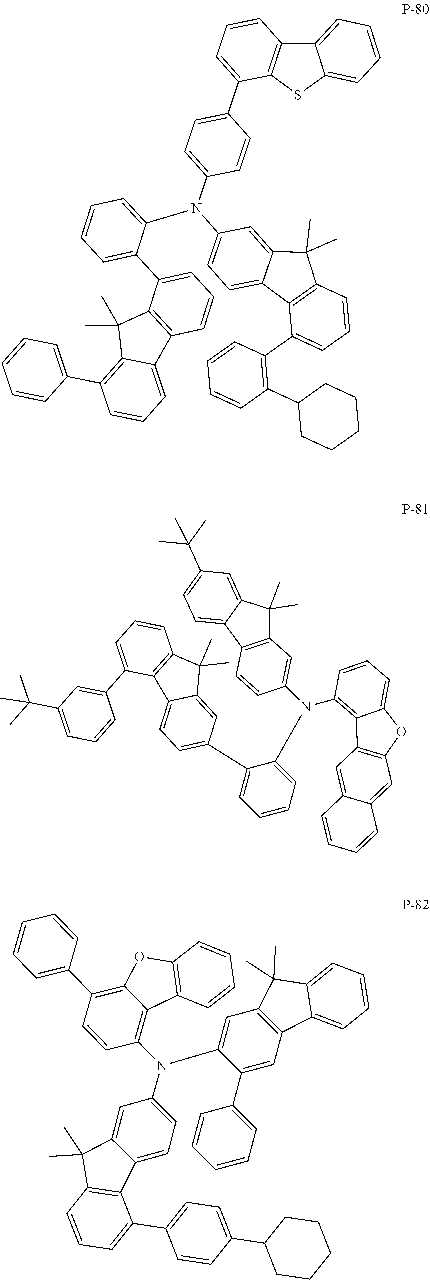 [SMILES: CC1(C)c2cc(N(c3ccc(-c4cccc5c4sc4ccccc45)cc3)c3ccccc3-c3cccc4c3C(C)(C)c3c(-c5ccccc5)cccc3-4)ccc2-c2c(-c3ccccc3C3CCCCC3)cccc21 ; CC(C)(C)c1cccc(-c2cccc3c2-c2ccc(-c4ccccc4N(c4ccc5c(c4)C(C)(C)c4cc(C(C)(C)C)ccc4-5)c4cccc5oc6cc7ccccc7cc6c45)cc2C3(C)C)c1 ; CC1(C)c2ccccc2-c2cc(-c3ccccc3)c(N(c3ccc4c(c3)C(C)(C)c3cccc(-c5ccc(C6CCCCC6)cc5)c3-4)c3ccc(-c4ccccc4)c4oc5ccccc5c34)cc21]

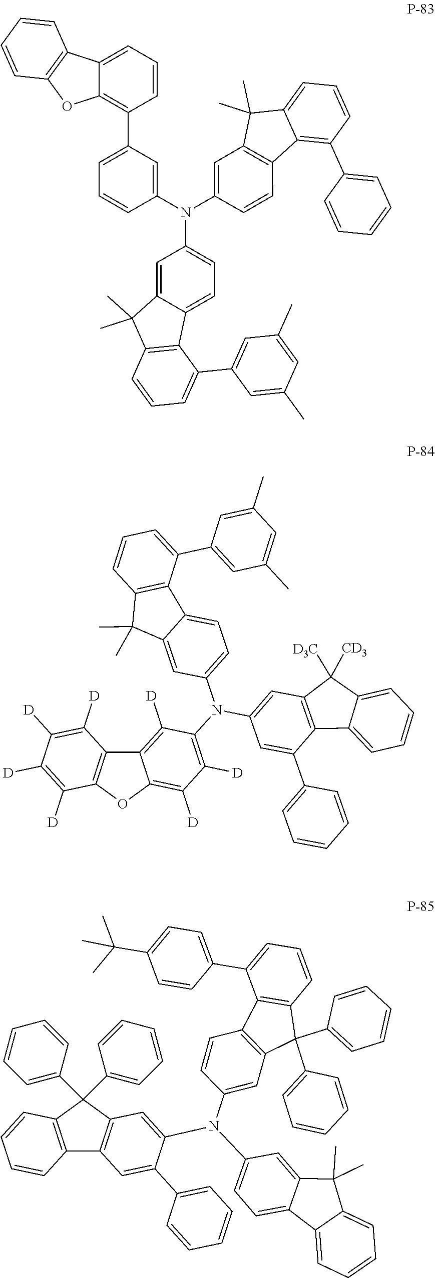 [SMILES: Cc1cc(C)cc(-c2cccc3c2-c2ccc(N(c4cccc(-c5cccc6c5oc5ccccc56)c4)c4ccc5c(c4)C(C)(C)c4cccc(-c6ccccc6)c4-5)cc2C3(C)C)c1 ; [2H]c1c([2H])c([2H])c2c(oc3c([2H])c([2H])c(N(c4ccc5c(c4)C(C)(C)c4cccc(-c6cc(C)cc(C)c6)c4-5)c4cc(-c5ccccc5)c5c(c4)C(C([2H])([2H])[2H])(C([2H])([2H])[2H])c4ccccc4-5)c([2H])c32)c1[2H] ; CC(C)(C)c1ccc(-c2cccc3c2-c2ccc(N(c4ccc5c(c4)C(C)(C)c4ccccc4-5)c4cc5c(cc4-c4ccccc4)-c4ccccc4C5(c4ccccc4)c4ccccc4)cc2C3(c2ccccc2)c2ccccc2)cc1]

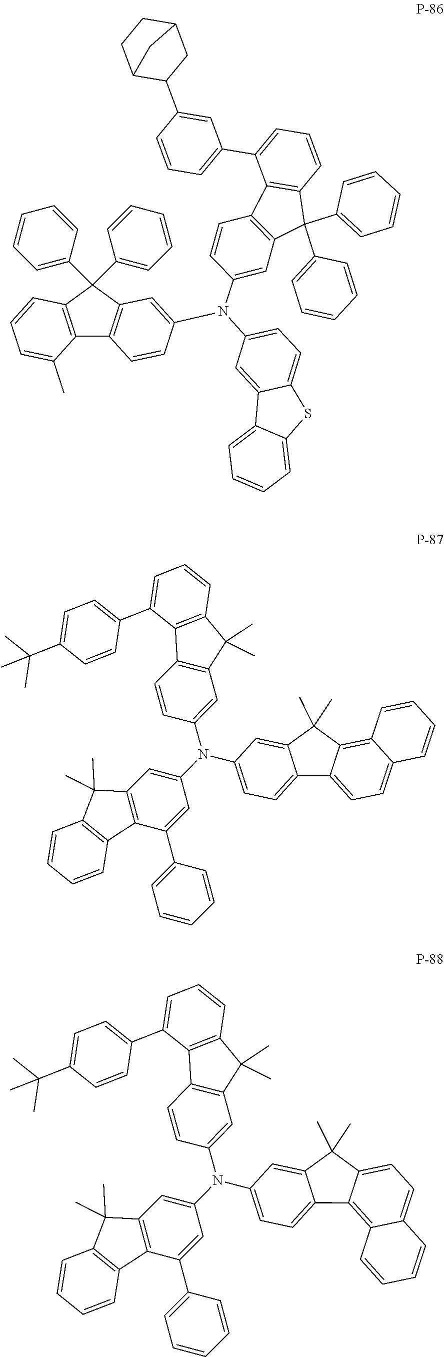 [SMILES: Cc1cccc2c1-c1ccc(N(c3ccc4c(c3)C(c3ccccc3)(c3ccccc3)c3cccc(-c5cccc(C6CC7CCC6C7)c5)c3-4)c3ccc4sc5ccccc5c4c3)cc1C2(c1ccccc1)c1ccccc1 ; CC(C)(C)c1ccc(-c2cccc3c2-c2ccc(N(c4ccc5c(c4)C(C)(C)c4c-5ccc5ccccc45)c4cc(-c5ccccc5)c5c(c4)C(C)(C)c4ccccc4-5)cc2C3(C)C)cc1 ; CC(C)(C)c1ccc(-c2cccc3c2-c2ccc(N(c4ccc5c(c4)C(C)(C)c4ccc6ccccc6c4-5)c4cc(-c5ccccc5)c5c(c4)C(C)(C)c4ccccc4-5)cc2C3(C)C)cc1]

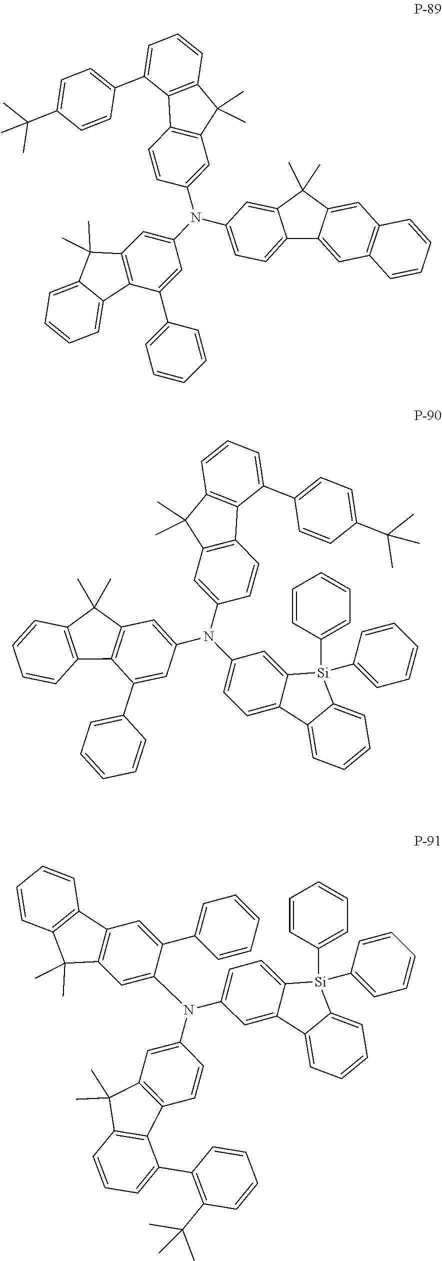 [SMILES: CC(C)(C)c1ccc(-c2cccc3c2-c2ccc(N(c4ccc5c(c4)C(C)(C)c4cc6ccccc6cc4-5)c4cc(-c5ccccc5)c5c(c4)C(C)(C)c4ccccc4-5)cc2C3(C)C)cc1 ; CC(C)(C)c1ccc(-c2cccc3c2-c2ccc(N(c4cc(-c5ccccc5)c5c(c4)C(C)(C)c4ccccc4-5)c4ccc5c(c4)[Si](c4ccccc4)(c4ccccc4)c4ccccc4-5)cc2C3(C)C)cc1 ; CC(C)(C)c1ccccc1-c1cccc2c1-c1ccc(N(c3ccc4c(c3)-c3ccccc3[Si]4(c3ccccc3)c3ccccc3)c3cc4c(cc3-c3ccccc3)-c3ccccc3C4(C)C)cc1C2(C)C]

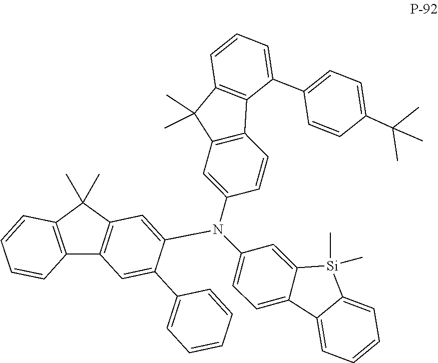 [SMILES: CC(C)(C)c1ccc(-c2cccc3c2-c2ccc(N(c4ccc5c(c4)[Si](C)(C)c4ccccc4-5)c4cc5c(cc4-c4ccccc4)-c4ccccc4C5(C)C)cc2C3(C)C)cc1]

In another aspect, the present invention provides an organic electronic device including a first electrode, a second electrode, and an organic layer formed between the first electrode and the second electrode, wherein the organic layer comprises the compound of Formula 1, and preferably the compound is included in a light-emitting auxiliary layer.

In another aspect, the present invention provides an electronic apparatus comprising a display device comprising an organic electronic device, and a control unit configured to drive the display device, wherein the organic electronic device comprises compound represented by Formula 1.

In yet another aspect, the present invention provides a compound represented by Formula 1, which is obtained by recovering and purifying the material of the organic layer from the deposition equipment after depositing the organic layer in the manufacturing process of the organic electronic device. The purity of the compound obtained by recovery and purification is 99.9% or more.

In another aspect, the present invention provides a method for recovering a compound represented by Formula 1, the method comprising: depositing an organic layer material comprising the compound represented by Formula 1; recovering the organic layer material that is attached to a deposition apparatus; and purifying the recovered organic layer material to obtain the compound represented by Formula 1 having a purity of 99.9% or higher.

The purification step may comprise: a recrystallization step in which the recovered material of an organic layer is recrystallized using a recrystallization solvent; an adsorption and separation step using an adsorbent; and a sublimation and purification step.

The recrystallization step may comprise a preliminary purification process in which a compound represented by Formula 1 having a purity of 98% is obtained using a recrystallization solvent.

As the recrystallization solvent, a polar solvent having a polarity index (PI) of 5.5 to 7.2 is preferably used, or a mixed solvent comprising a polar solvent having a polarity index of 5.5 to 7.2 and a non-polar solvent having a polarity index of 2.0 to 4.7 may be used.

When a mixed solvent of a polar solvent and a non-polar solvent is used as the recrystallization solvent, the non-polar solvent may be used in an amount of 15% (v/v) or less relative to the volume of the polar solvent.

As the recrystallization solvent, a single solvent of methylpyrrolidone (N-methylpyrrolidone; NMP) is preferably used. Alternatively, a mixed polar solvent in which methylpyrrolidone is mixed with at least one solvent selected from the group consisting of 1,3-dimethyl-2-imidazolidinone, 2-pyrrolidone, N,N-dimethylformamide, dimethylacetamide, and dimethyl sulfoxide may be used. In addition, a single or mixed non-polar solvent selected from the group consisting of toluene, dichloromethane (DCM), dichloroethane (DCE), tetrahydrofuran (THF), chloroform, ethyl acetate, and butanone, or a mixture of the polar solvent and the non-polar solvent may also be used.

The preliminary purification process may comprise dissolving the unpurified organic electroluminescent material, which has been recovered from a deposition apparatus, in a polar solvent at a temperature of 90° C. to 120° C., and cooling the solution to a temperature of 0° C. to 5° C. to precipitate crystals.

The preliminary purification process may comprise dissolving the unpurified organic electroluminescent material, recovered from a deposition apparatus, in a polar solvent at a temperature of 90° C. to 120° C.; cooling the solution to a temperature of 35° C. to 40° C.; adding a non-polar solvent to the cooled solution; and further cooling the resulting mixture to a temperature of 0° C. to 5° C. to precipitate crystals.

The preliminary purification process may comprise dissolving the unpurified organic electroluminescent material, recovered from a deposition apparatus, in a non-polar solvent, and concentrating the solution to remove the non-polar solvent while precipitating crystals.

The preliminary purification process may comprise a step of recrystallization using a polar solvent, followed by a subsequent recrystallization step using a non-polar solvent.

In the adsorption separation step using an adsorbent, the adsorbent may be selected from the group consisting of activated carbon, silica gel, alumina, and other materials known for use in adsorption.

Hereinafter, the present invention will be described in further detail with reference to specific examples regarding the synthesis of compound represented by Formula 1 and the fabrication of an organic electronic device. However, the present invention is not limited to the following examples.

### Synthesis Example

The compound (final products) represented by Formula 1 according to the present invention can be synthesized by reacting Sub 1 and Sub 2 as shown in Reaction Formula 1 below, but is not limited thereto.

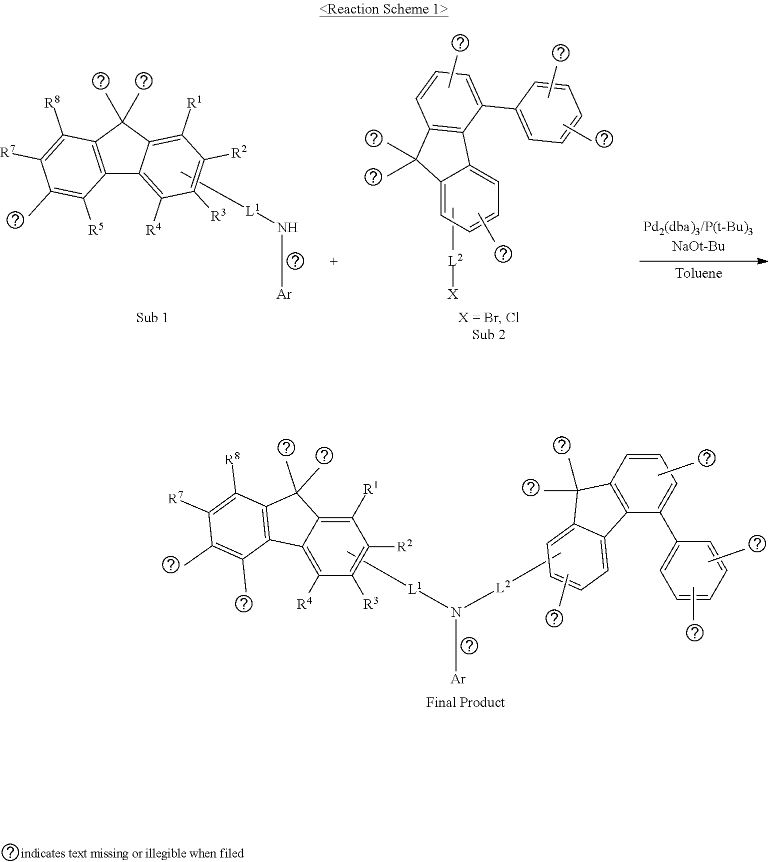

### Exemplary Compounds of Sub 1

The compound belonging to Sub 1 may be, but is not limited to, a compound such as those listed below, and the FD-MS (Field Desorption-Mass Spectrometry) values of the compounds are as shown in Table 1 below.

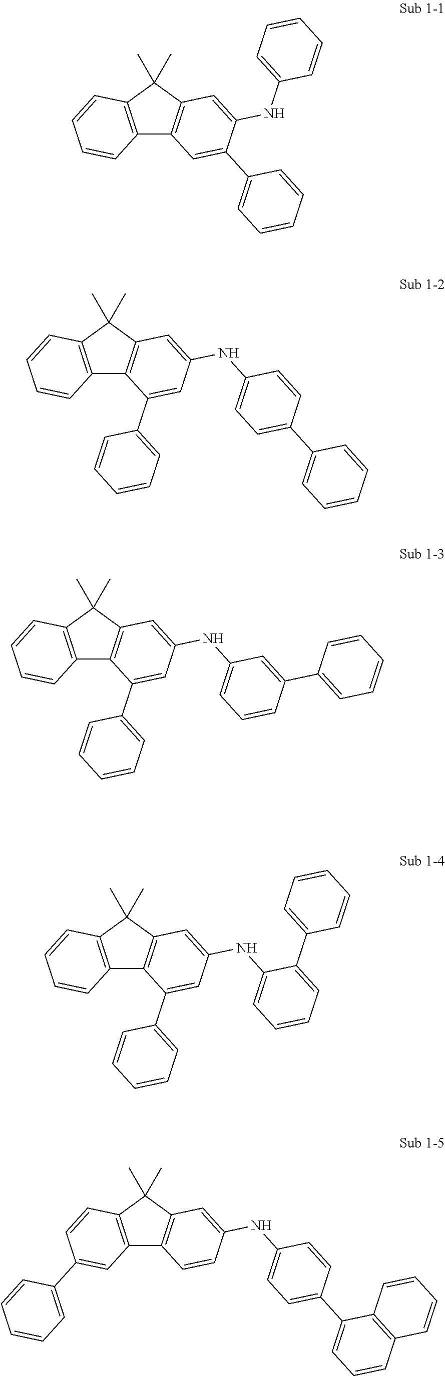 [SMILES: CC1(C)c2ccccc2-c2cc(-c3ccccc3)c(Nc3ccccc3)cc21 ; CC1(C)c2ccccc2-c2c(-c3ccccc3)cc(Nc3ccc(-c4ccccc4)cc3)cc21 ; CC1(C)c2ccccc2-c2c(-c3ccccc3)cc(Nc3cccc(-c4ccccc4)c3)cc21 ; CC1(C)c2ccccc2-c2c(-c3ccccc3)cc(Nc3ccccc3-c3ccccc3)cc21 ; CC1(C)c2ccc(-c3ccccc3)cc2-c2ccc(Nc3ccc(-c4cccc5ccccc45)cc3)cc21]

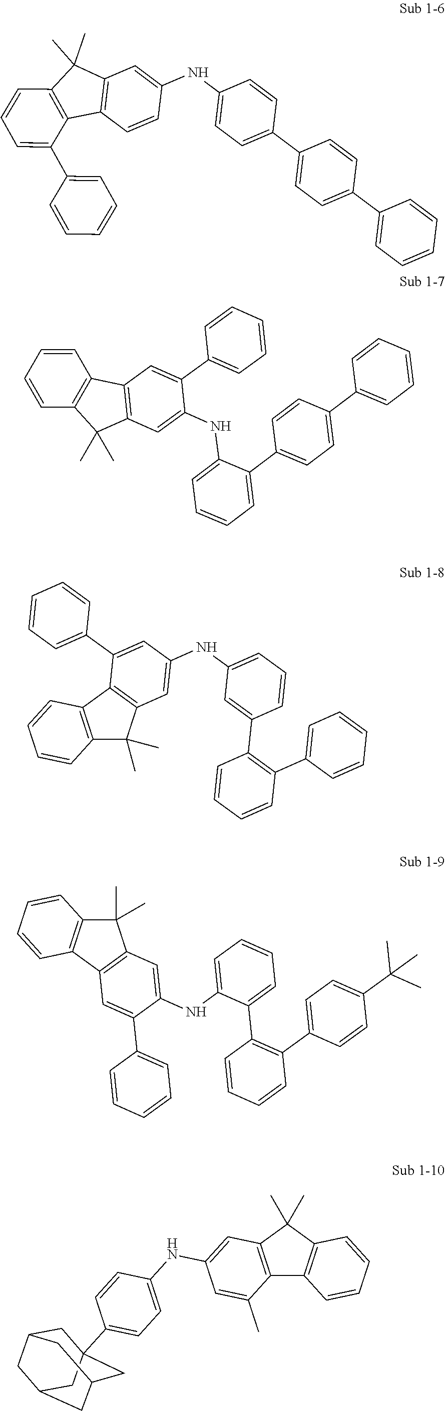 [SMILES: CC1(C)c2cc(Nc3ccc(-c4ccc(-c5ccccc5)cc4)cc3)ccc2-c2c(-c3ccccc3)cccc21 ; CC1(C)c2ccccc2-c2cc(-c3ccccc3)c(Nc3ccccc3-c3ccc(-c4ccccc4)cc3)cc21 ; CC1(C)c2ccccc2-c2c(-c3ccccc3)cc(Nc3cccc(-c4ccccc4-c4ccccc4)c3)cc21 ; CC(C)(C)c1ccc(-c2ccccc2-c2ccccc2Nc2cc3c(cc2-c2ccccc2)-c2ccccc2C3(C)C)cc1 ; Cc1cc(Nc2ccc(C34CC5CC(CC(C5)C3)C4)cc2)cc2c1-c1ccccc1C2(C)C]

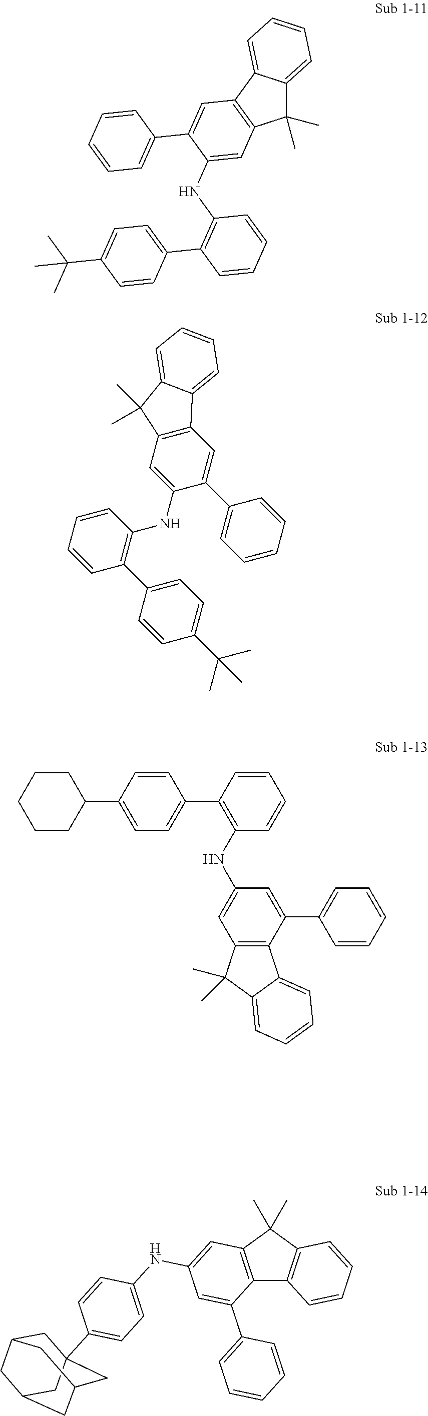 [SMILES: CC(C)(C)c1ccc(-c2ccccc2Nc2cc3c(cc2-c2ccccc2)-c2ccccc2C3(C)C)cc1 ; CC(C)(C)c1ccc(-c2ccccc2Nc2cc3c(cc2-c2ccccc2)-c2ccccc2C3(C)C)cc1 ; CC1(C)c2ccccc2-c2c(-c3ccccc3)cc(Nc3ccccc3-c3ccc(C4CCCCC4)cc3)cc21 ; CC1(C)c2ccccc2-c2c(-c3ccccc3)cc(Nc3ccc(C45CC6CC(CC(C6)C4)C5)cc3)cc21]

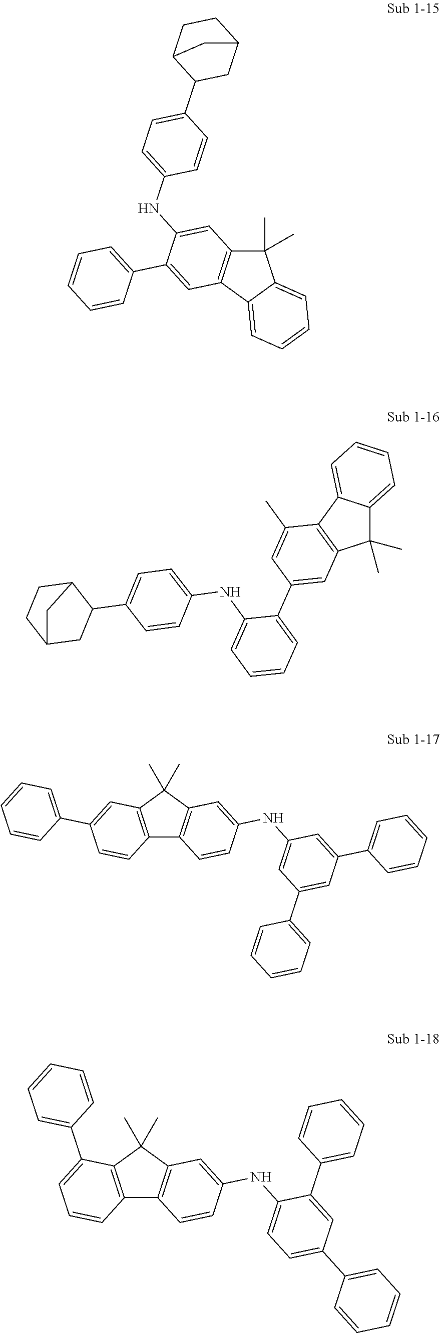 [SMILES: CC1(C)c2ccccc2-c2cc(-c3ccccc3)c(Nc3ccc(C4CC5CCC4C5)cc3)cc21 ; Cc1cc(-c2ccccc2Nc2ccc(C3CC4CCC3C4)cc2)cc2c1-c1ccccc1C2(C)C ; CC1(C)c2cc(Nc3cc(-c4ccccc4)cc(-c4ccccc4)c3)ccc2-c2ccc(-c3ccccc3)cc21 ; CC1(C)c2cc(Nc3ccc(-c4ccccc4)cc3-c3ccccc3)ccc2-c2cccc(-c3ccccc3)c21]

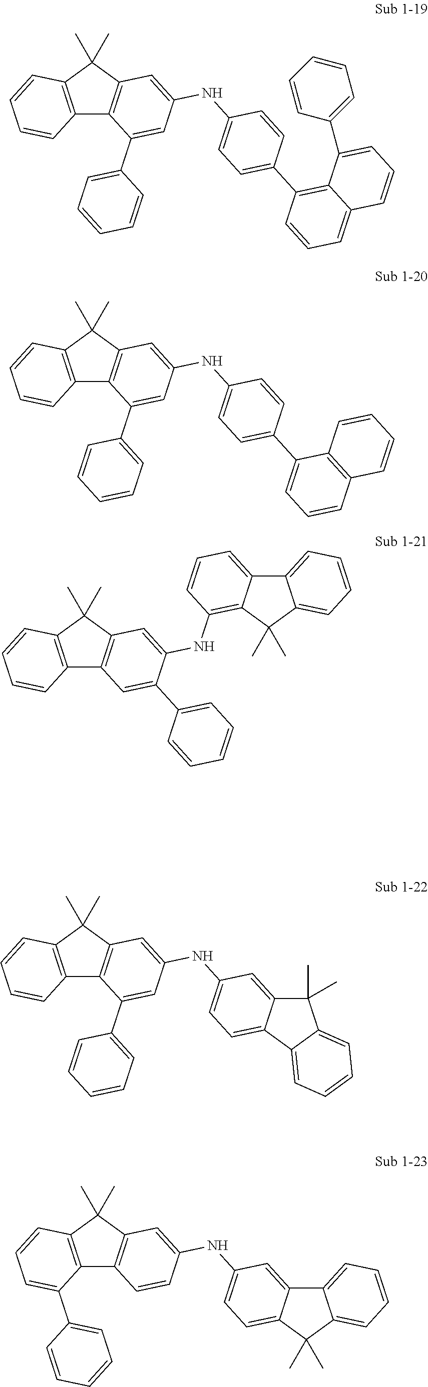

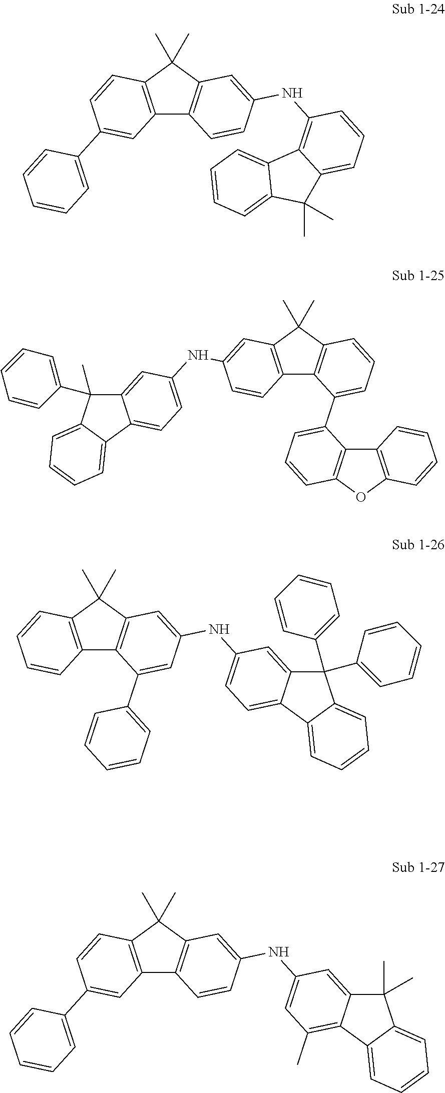 [SMILES: CC1(C)c2ccc(-c3ccccc3)cc2-c2ccc(Nc3cccc4c3-c3ccccc3C4(C)C)cc21 ; CC1(C)c2cc(Nc3ccc4c(c3)C(C)(c3ccccc3)c3ccccc3-4)ccc2-c2c(-c3cccc4oc5ccccc5c34)cccc21 ; CC1(C)c2ccccc2-c2c(-c3ccccc3)cc(Nc3ccc4c(c3)C(c3ccccc3)(c3ccccc3)c3ccccc3-4)cc21 ; Cc1cc(Nc2ccc3c(c2)C(C)(C)c2ccc(-c4ccccc4)cc2-3)cc2c1-c1ccccc1C2(C)C]

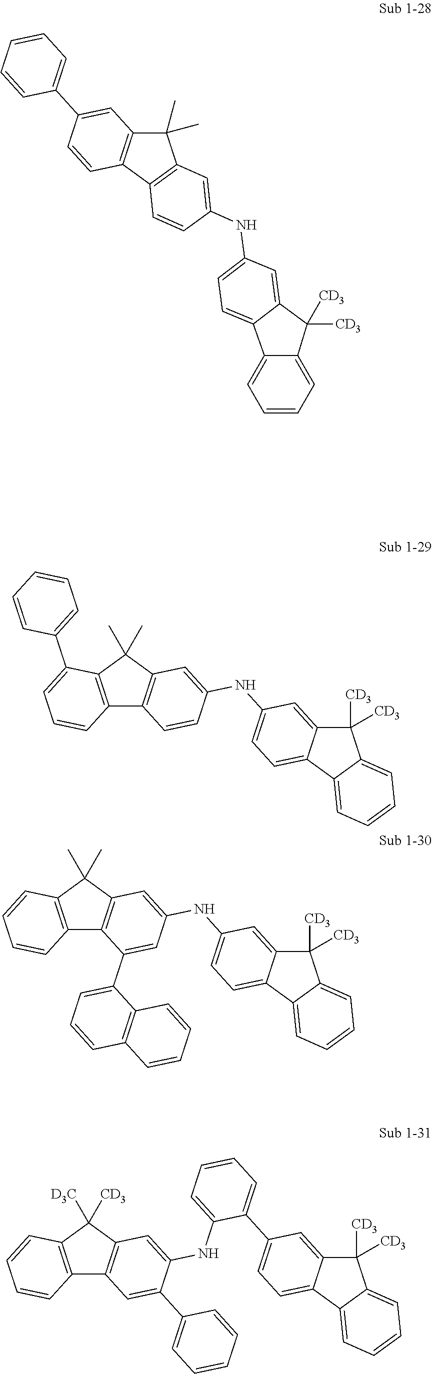 [SMILES: [2H]C([2H])([2H])C1(C([2H])([2H])[2H])c2ccccc2-c2ccc(Nc3ccc4c(c3)C(C)(C)c3cc(-c5ccccc5)ccc3-4)cc21 ; [2H]C([2H])([2H])C1(C([2H])([2H])[2H])c2ccccc2-c2ccc(Nc3ccc4c(c3)C(C)(C)c3c(-c5ccccc5)cccc3-4)cc21 ; [2H]C([2H])([2H])C1(C([2H])([2H])[2H])c2ccccc2-c2ccc(Nc3cc(-c4cccc5ccccc45)c4c(c3)C(C)(C)c3ccccc3-4)cc21 ; [2H]C([2H])([2H])C1(C([2H])([2H])[2H])c2ccccc2-c2ccc(-c3ccccc3Nc3cc4c(cc3-c3ccccc3)-c3ccccc3C4(C([2H])([2H])[2H])C([2H])([2H])[2H])cc21]

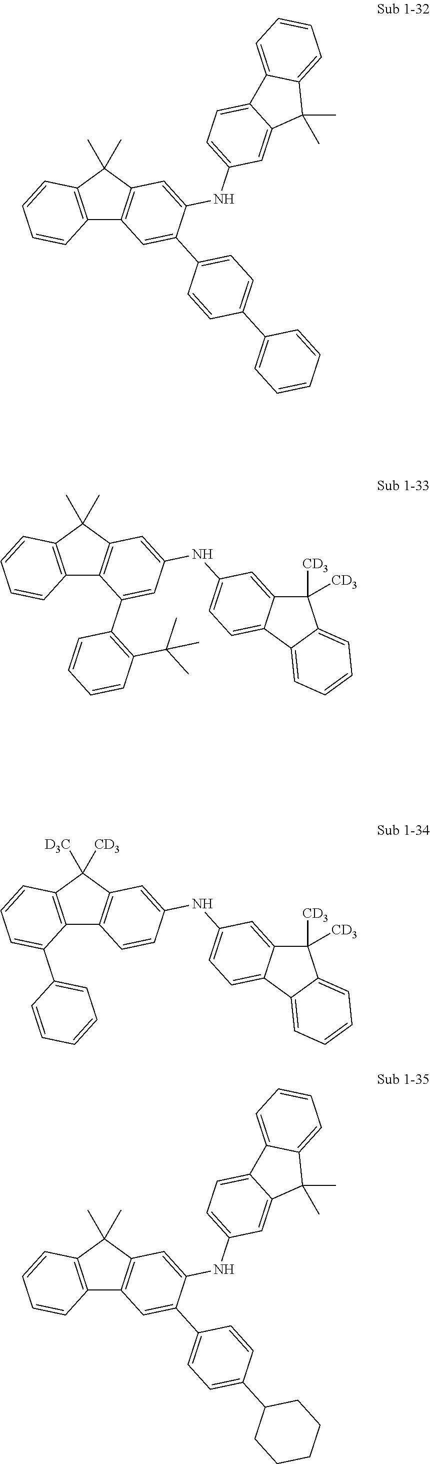 [SMILES: CC1(C)c2ccccc2-c2ccc(Nc3cc4c(cc3-c3ccc(-c5ccccc5)cc3)-c3ccccc3C4(C)C)cc21 ; [2H]C([2H])([2H])C1(C([2H])([2H])[2H])c2ccccc2-c2ccc(Nc3cc(-c4ccccc4C(C)(C)C)c4c(c3)C(C)(C)c3ccccc3-4)cc21 ; [2H]C([2H])([2H])C1(C([2H])([2H])[2H])c2ccccc2-c2ccc(Nc3ccc4c(c3)C(C([2H])([2H])[2H])(C([2H])([2H])[2H])c3cccc(-c5ccccc5)c3-4)cc21 ; CC1(C)c2ccccc2-c2ccc(Nc3cc4c(cc3-c3ccc(C5CCCCC5)cc3)-c3ccccc3C4(C)C)cc21]

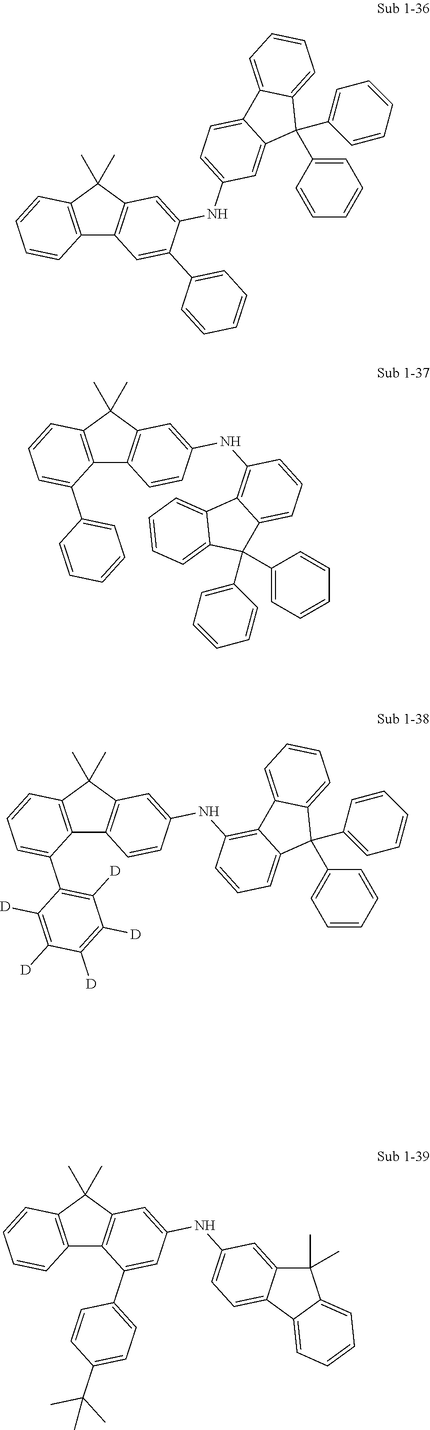 [SMILES: CC1(C)c2ccccc2-c2cc(-c3ccccc3)c(Nc3ccc4c(c3)C(c3ccccc3)(c3ccccc3)c3ccccc3-4)cc21 ; CC1(C)c2cc(Nc3cccc4c3-c3ccccc3C4(c3ccccc3)c3ccccc3)ccc2-c2c(-c3ccccc3)cccc21 ; [2H]c1c([2H])c([2H])c(-c2cccc3c2-c2ccc(Nc4cccc5c4-c4ccccc4C5(c4ccccc4)c4ccccc4)cc2C3(C)C)c([2H])c1[2H] ; CC(C)(C)c1ccc(-c2cc(Nc3ccc4c(c3)C(C)(C)c3ccccc3-4)cc3c2-c2ccccc2C3(C)C)cc1]

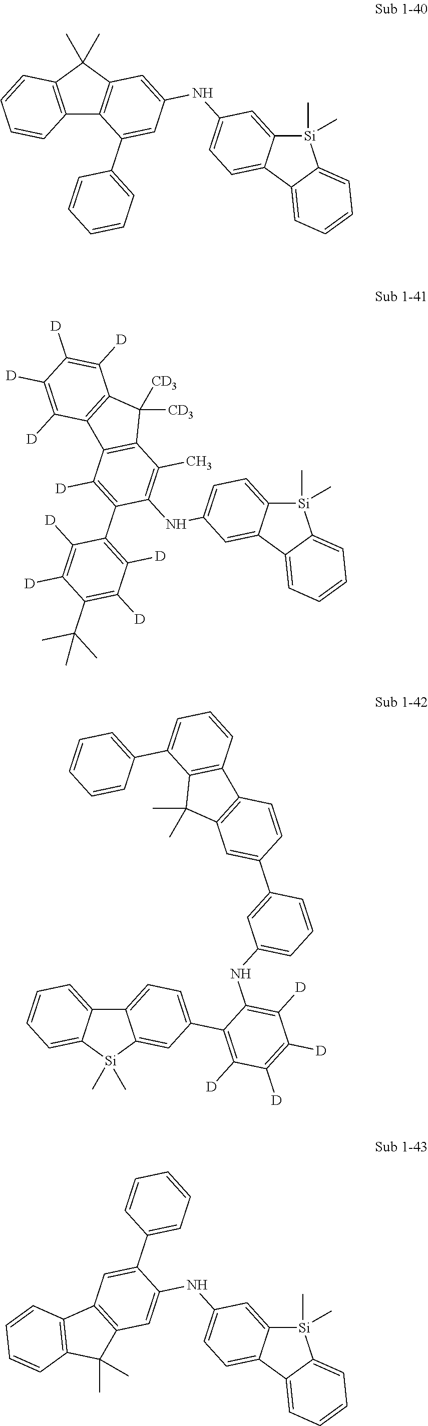 [SMILES: CC1(C)c2ccccc2-c2c(-c3ccccc3)cc(Nc3ccc4c(c3)[Si](C)(C)c3ccccc3-4)cc21 ; [2H]c1c([2H])c([2H])c2c(c1[2H])-c1c([2H])c(-c3c([2H])c([2H])c(C(C)(C)C)c([2H])c3[2H])c(Nc3ccc4c(c3)-c3ccccc3[Si]4(C)C)c(C)c1C2(C([2H])([2H])[2H])C([2H])([2H])[2H] ; [2H]c1c([2H])c([2H])c(-c2ccc3c(c2)[Si](C)(C)c2ccccc2-3)c(Nc2cccc(-c3ccc4c(c3)C(C)(C)c3c(-c5ccccc5)cccc3-4)c2)c1[2H] ; CC1(C)c2ccccc2-c2cc(-c3ccccc3)c(Nc3ccc4c(c3)[Si](C)(C)c3ccccc3-4)cc21]

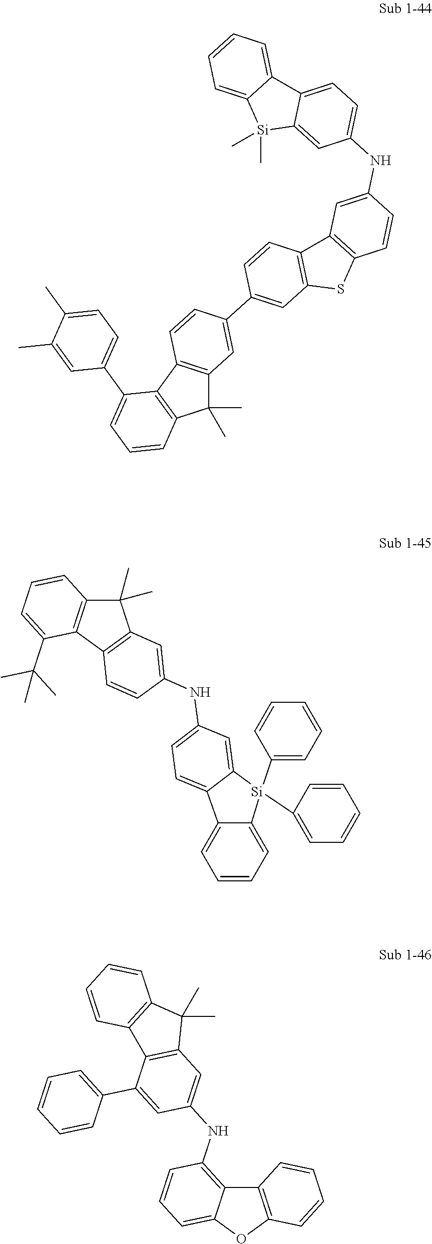 [SMILES: Cc1ccc(-c2cccc3c2-c2ccc(-c4ccc5c(c4)sc4ccc(Nc6ccc7c(c6)[Si](C)(C)c6ccccc6-7)cc45)cc2C3(C)C)cc1C ; CC(C)(C)c1cccc2c1-c1ccc(Nc3ccc4c(c3)[Si](c3ccccc3)(c3ccccc3)c3ccccc3-4)cc1C2(C)C ; CC1(C)c2ccccc2-c2c(-c3ccccc3)cc(Nc3cccc4oc5ccccc5c34)cc21]

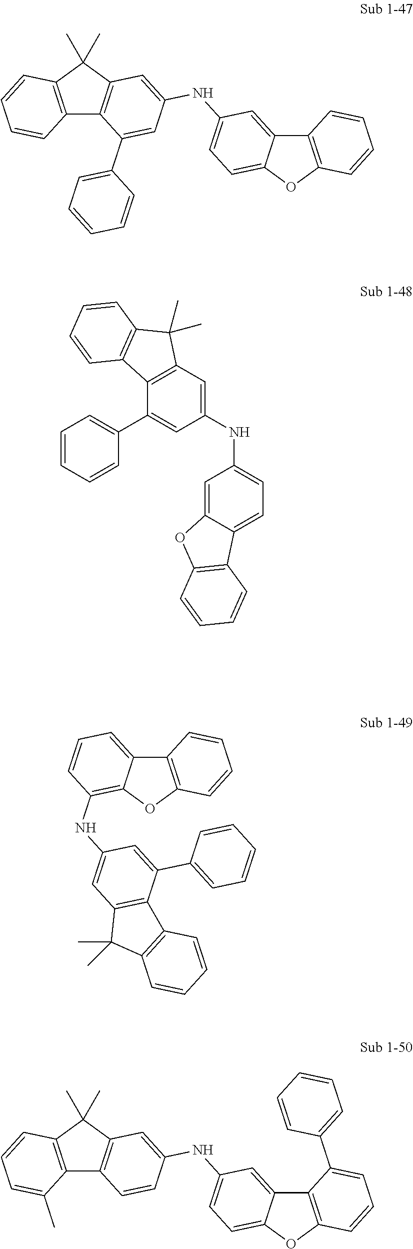 [SMILES: CC1(C)c2ccccc2-c2c(-c3ccccc3)cc(Nc3ccc4oc5ccccc5c4c3)cc21 ; CC1(C)c2ccccc2-c2c(-c3ccccc3)cc(Nc3ccc4c(c3)oc3ccccc34)cc21 ; CC1(C)c2ccccc2-c2c(-c3ccccc3)cc(Nc3cccc4c3oc3ccccc34)cc21 ; Cc1cccc2c1-c1ccc(Nc3ccc4oc5cccc(-c6ccccc6)c5c4c3)cc1C2(C)C]

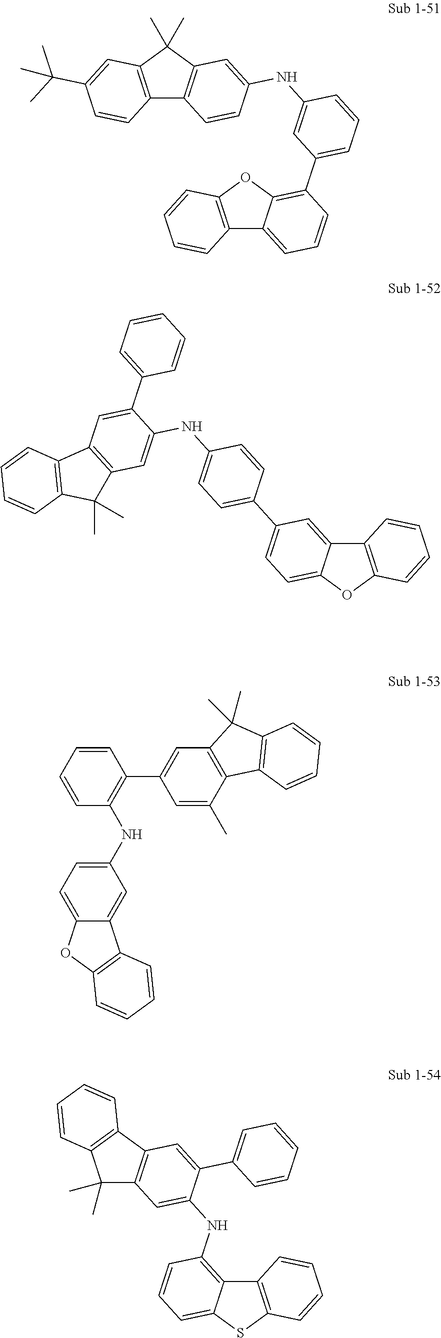 [SMILES: CC(C)(C)c1ccc2c(c1)C(C)(C)c1cc(Nc3cccc(-c4cccc5c4oc4ccccc45)c3)ccc1-2 ; CC1(C)c2ccccc2-c2cc(-c3ccccc3)c(Nc3ccc(-c4ccc5oc6ccccc6c5c4)cc3)cc21 ; Cc1cc(-c2ccccc2Nc2ccc3oc4ccccc4c3c2)cc2c1-c1ccccc1C2(C)C ; CC1(C)c2ccccc2-c2cc(-c3ccccc3)c(Nc3cccc4sc5ccccc5c34)cc21]

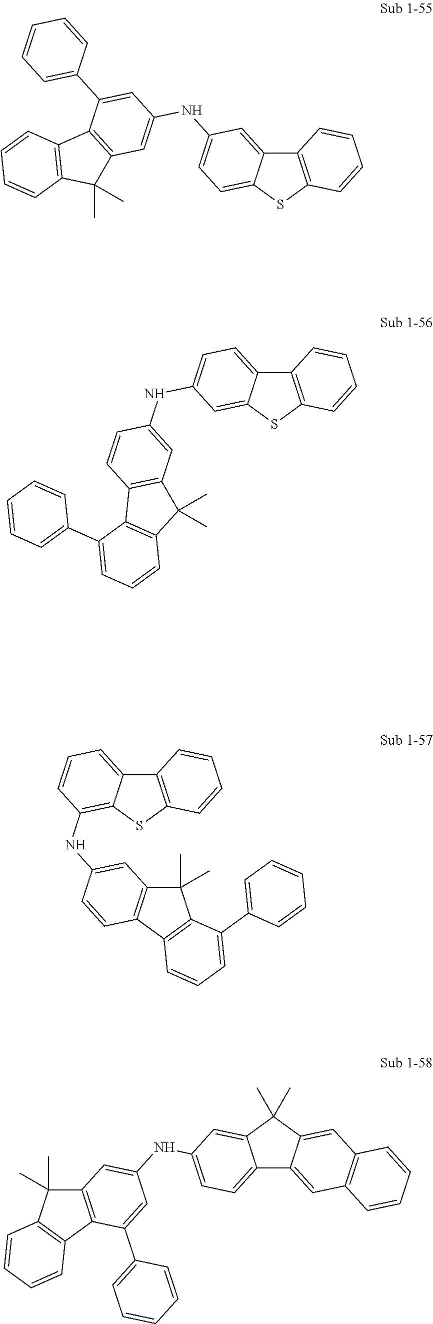 [SMILES: CC1(C)c2ccccc2-c2c(-c3ccccc3)cc(Nc3ccc4sc5ccccc5c4c3)cc21 ; CC1(C)c2cc(Nc3ccc4c(c3)sc3ccccc34)ccc2-c2c(-c3ccccc3)cccc21 ; CC1(C)c2cc(Nc3cccc4c3sc3ccccc34)ccc2-c2cccc(-c3ccccc3)c21 ; CC1(C)c2cc(Nc3cc(-c4ccccc4)c4c(c3)C(C)(C)c3ccccc3-4)ccc2-c2cc3ccccc3cc21]

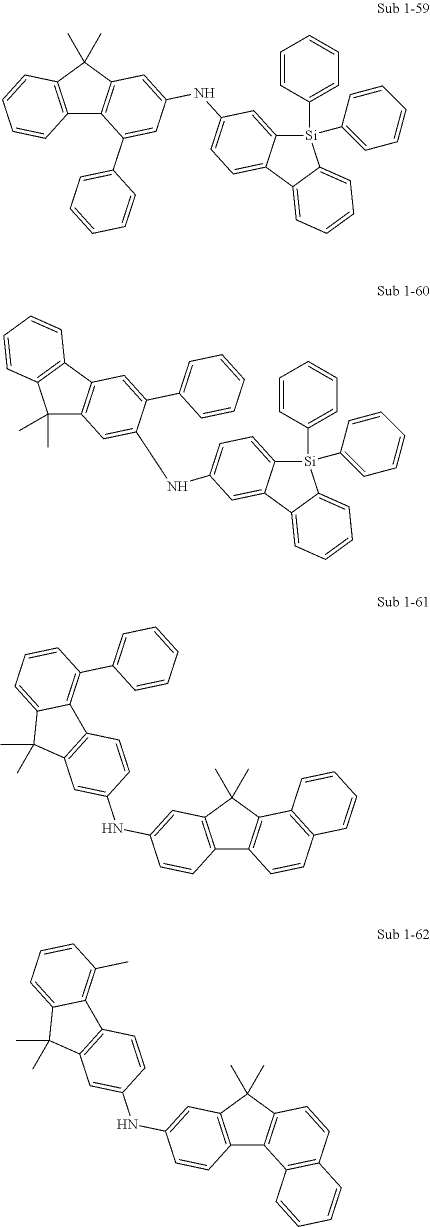 [SMILES: CC1(C)c2ccccc2-c2c(-c3ccccc3)cc(Nc3ccc4c(c3)[Si](c3ccccc3)(c3ccccc3)c3ccccc3-4)cc21 ; CC1(C)c2ccccc2-c2cc(-c3ccccc3)c(Nc3ccc4c(c3)-c3ccccc3[Si]4(c3ccccc3)c3ccccc3)cc21 ; CC1(C)c2cc(Nc3ccc4c(c3)C(C)(C)c3c-4ccc4ccccc34)ccc2-c2c(-c3ccccc3)cccc21 ; Cc1cccc2c1-c1ccc(Nc3ccc4c(c3)C(C)(C)c3ccc5ccccc5c3-4)cc1C2(C)C]

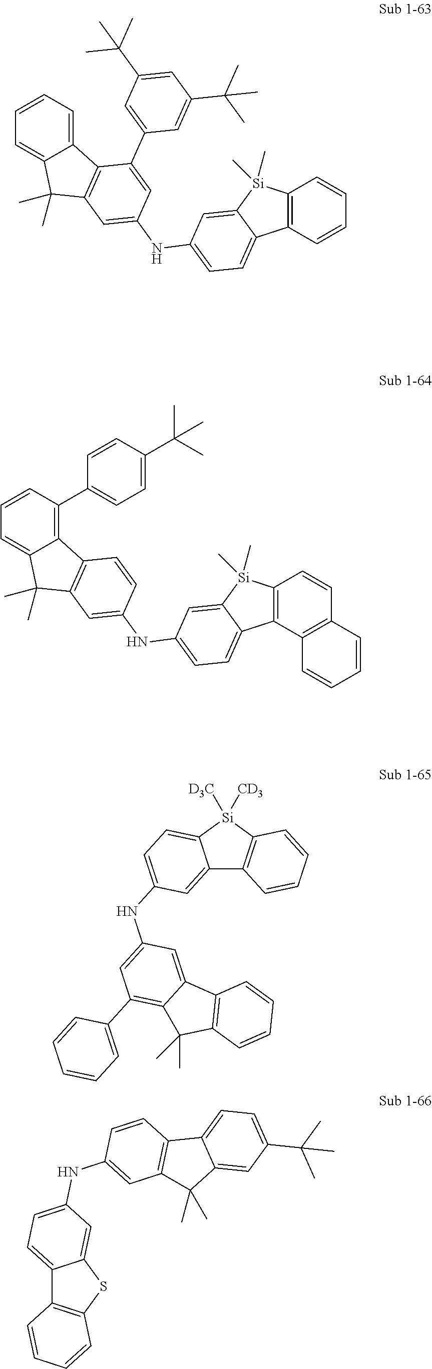 [SMILES: CC(C)(C)c1cc(-c2cc(Nc3ccc4c(c3)[Si](C)(C)c3ccccc3-4)cc3c2-c2ccccc2C3(C)C)cc(C(C)(C)C)c1 ; CC(C)(C)c1ccc(-c2cccc3c2-c2ccc(Nc4ccc5c(c4)[Si](C)(C)c4ccc6ccccc6c4-5)cc2C3(C)C)cc1 ; [2H]C([2H])([2H])[Si]1(C([2H])([2H])[2H])c2ccccc2-c2cc(Nc3cc(-c4ccccc4)c4c(c3)-c3ccccc3C4(C)C)ccc21 ; CC(C)(C)c1ccc2c(c1)C(C)(C)c1cc(Nc3ccc4c(c3)sc3ccccc34)ccc1-2]

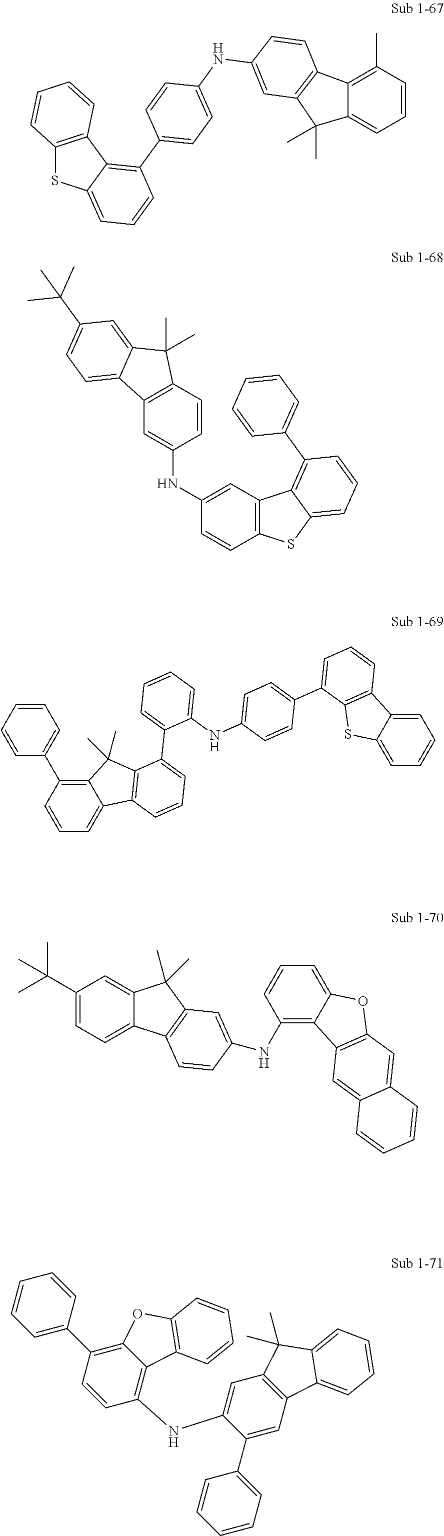 [SMILES: Cc1cccc2c1-c1ccc(Nc3ccc(-c4cccc5sc6ccccc6c45)cc3)cc1C2(C)C ; CC(C)(C)c1ccc2c(c1)C(C)(C)c1ccc(Nc3ccc4sc5cccc(-c6ccccc6)c5c4c3)cc1-2 ; CC1(C)c2c(-c3ccccc3)cccc2-c2cccc(-c3ccccc3Nc3ccc(-c4cccc5c4sc4ccccc45)cc3)c21 ; CC(C)(C)c1ccc2c(c1)C(C)(C)c1cc(Nc3cccc4oc5cc6ccccc6cc5c34)ccc1-2 ; CC1(C)c2ccccc2-c2cc(-c3ccccc3)c(Nc3ccc(-c4ccccc4)c4oc5ccccc5c34)cc21]

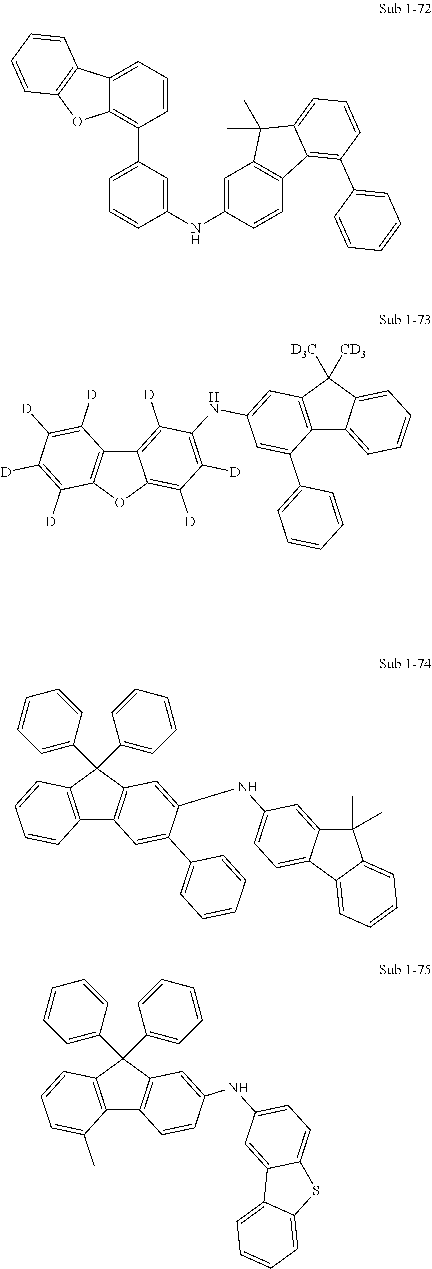 [SMILES: CC1(C)c2cc(Nc3cccc(-c4cccc5c4oc4ccccc45)c3)ccc2-c2c(-c3ccccc3)cccc21 ; [2H]c1c([2H])c([2H])c2c(oc3c([2H])c([2H])c(Nc4cc(-c5ccccc5)c5c(c4)C(C([2H])([2H])[2H])(C([2H])([2H])[2H])c4ccccc4-5)c([2H])c32)c1[2H] ; CC1(C)c2ccccc2-c2ccc(Nc3cc4c(cc3-c3ccccc3)-c3ccccc3C4(c3ccccc3)c3ccccc3)cc21 ; Cc1cccc2c1-c1ccc(Nc3ccc4sc5ccccc5c4c3)cc1C2(c1ccccc1)c1ccccc1]

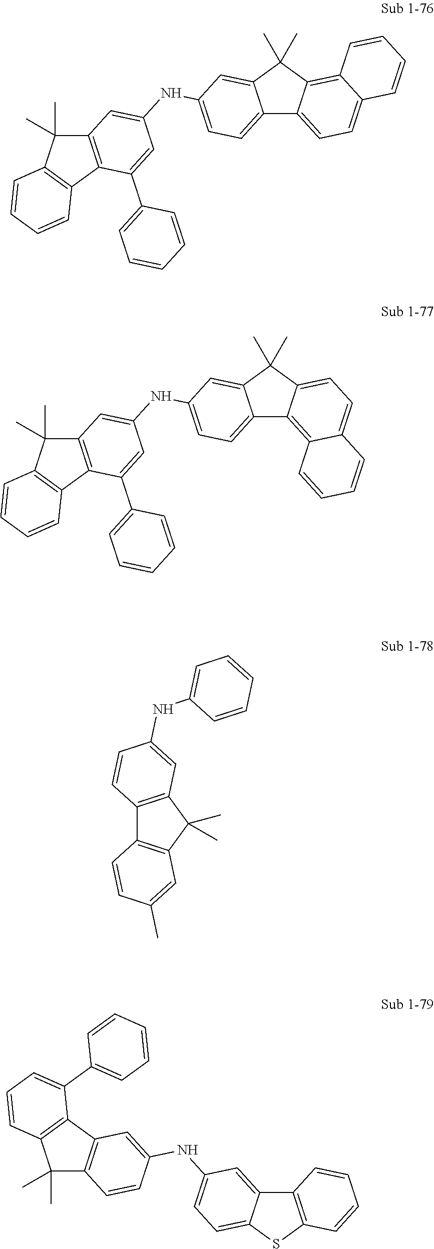 [SMILES: CC1(C)c2ccccc2-c2c(-c3ccccc3)cc(Nc3ccc4c(c3)C(C)(C)c3c-4ccc4ccccc34)cc21 ; CC1(C)c2ccccc2-c2c(-c3ccccc3)cc(Nc3ccc4c(c3)C(C)(C)c3ccc5ccccc5c3-4)cc21 ; Cc1ccc2c(c1)C(C)(C)c1cc(Nc3ccccc3)ccc1-2 ; CC1(C)c2ccc(Nc3ccc4sc5ccccc5c4c3)cc2-c2c(-c3ccccc3)cccc21]

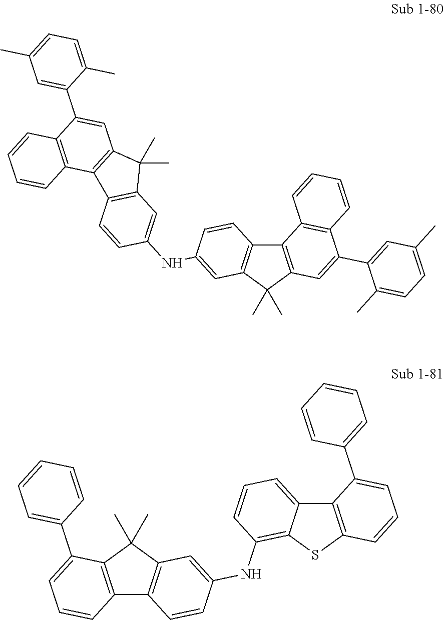

TABLE 1

| Compound | FD-MS | Compound | FD-MS |
|---|---|---|---|
| Sub 1-1 | m/z = 361.18(C₂₇H₂₃N = 361.49) | Sub 1-2 | m/z = 437.21(C₃₃H₂₇N = 437.59) |
| Sub 1-3 | m/z = 437.21(C₃₃H₂₇N = 437.59) | Sub 1-4 | m/z = 437.21(C₃₃H₂₇N = 437.59) |
| Sub 1-5 | m/z = 487.23(C₃₇H₂₉N = 487.65) | Sub 1-6 | m/z = 513.25(C₃₉H₃₁N = 513.68) |
| Sub 1-7 | m/z = 513.25(C₃₉H₃₁N = 513.68) | Sub 1-8 | m/z = 513.25(C₃₉H₃₁N = 513.68) |
| Sub 1-9 | m/z = 569.31(C₄₃H₃₉N = 569.79) | Sub 1-10 | m/z = 433.28(C₃₂H₃₅N = 433.64) |
| Sub 1-11 | m/z = 493.28(C₃₇H₃₅N = 493.69) | Sub 1-12 | m/z = 493.28(C₃₇H₃₅N = 493.69) |
| Sub 1-13 | m/z = 519.29(C₃₉H₃₇N = 519.73) | Sub 1-14 | m/z = 495.29(C₃₇H₃₇N = 495.71) |
| Sub 1-15 | m/z = 455.26(C₃₄H₃₃N = 455.65) | Sub 1-16 | m/z = 469.28(C₃₅H₃₅N = 469.67) |
| Sub 1-17 | m/z = 513.25(C₃₉H₃₁N = 513.68) | Sub 1-18 | m/z = 513.25(C₃₉H₃₁N = 513.68) |
| Sub 1-19 | m/z = 563.26(C₄₃H₃₃N = 563.74) | Sub 1-20 | m/z = 487.23(C₃₇H₂₉N = 487.65) |
| Sub 1-21 | m/z = 477.25(C₃₆H₃₁N = 477.65) | Sub 1-22 | m/z = 477.25(C₃₆H₃₁N = 477.65) |
| Sub 1-23 | m/z = 477.25(C₃₆H₃₁N = 477.65) | Sub 1-24 | m/z = 477.25(C₃₆H₃₁N = 477.65) |
| Sub 1-25 | m/z = 629.27(C₄₇H₃₅NO = 629.8) | Sub 1-26 | m/z = 601.28(C₄₆H₃₅N = 601.79) |
| Sub 1-27 | m/z = 491.26(C₃₇H₃₃N = 491.68) | Sub 1-28 | m/z = 483.28(C₃₆H₂₅D₆N = 483.69) |
| Sub 1-29 | m/z = 483.28(C₃₆H₂₅D₆N = 483.69) | Sub 1-30 | m/z = 533.3(C₄₀H₂₇D₆N = 533.75) |
| Sub 1-31 | m/z = 565.35(C₄₂H₂₃D₁₂N = 565.82) | Sub 1-32 | m/z = 553.28(C₄₂H₃₅N = 553.75) |
| Sub 1-33 | m/z = 539.35(C₄₀H₃₃D₆N = 539.8) | Sub 1-34 | m/z = 489.32(C₃₆H₁₉D₁₂N = 489.72) |
| Sub 1-35 | m/z = 559.32(C₄₂H₄₁N = 559.8) | Sub 1-36 | m/z = 601.28(C₄₆H₃₅N = 601.79) |
| Sub 1-37 | m/z = 601.28(C₄₆H₃₅N = 601.79) | Sub 1-38 | m/z = 606.31(C₄₆H₃₀D₅N = 606.82) |
| Sub 1-39 | m/z = 533.31(C₄₀H₃₉N = 533.76) | Sub 1-40 | m/z = 493.22(C₃₅H₃₁NSi = 493.73) |
| Sub 1-41 | m/z = 578.39(C₄₀H₂₆D₁₅NSi = 578.95) | Sub 1-42 | m/z = 649.31(C₄₇H₃₅D₄NSi = 649.95) |
| Sub 1-43 | m/z = 493.22(C₃₅H₃₁NSi = 493.73) | Sub 1-44 | m/z = 703.27(C₄₉H₄₁NSSi = 704.02) |
| Sub 1-45 | m/z = 597.29(C₄₃H₃₉NSi = 597.88) | Sub 1-46 | m/z = 451.19(C₃₃H₂₅NO = 451.57) |
| Sub 1-47 | m/z = 451.19(C₃₃H₂₅NO = 451.57) | Sub 1-48 | m/z = 451.19(C₃₃H₂₅NO = 451.57) |
| Sub 1-49 | m/z = 451.19(C₃₃H₂₅NO = 451.57) | Sub 1-50 | m/z = 465.21(C₃₄H₂₇NO = 465.6) |
| Sub 1-51 | m/z = 507.26(C₃₇H₃₃NO = 507.68) | Sub 1-52 | m/z = 527.22(C₃₉H₂₉NO = 527.67) |
| Sub 1-53 | m/z = 465.21(C₃₄H₂₇NO = 465.6) | Sub 1-54 | m/z = 467.17(C₃₃H₂₅NS = 467.63) |
| Sub 1-55 | m/z = 467.17(C₃₃H₂₅NS = 467.63) | Sub 1-56 | m/z = 467.17(C₃₃H₂₅NS = 467.63) |
| Sub 1-57 | m/z = 467.17(C₃₃H₂₅NS = 467.63) | Sub 1-58 | m/z = 527.26(C₄₀H₃₃N = 527.71) |
| Sub 1-59 | m/z = 617.25(C₄₅H₃₅NSi = 617.87) | Sub 1-60 | m/z = 617.25(C₄₅H₃₅NSi = 617.87) |
| Sub 1-61 | m/z = 527.26(C₄₀H₃₃N = 527.71) | Sub 1-62 | m/z = 465.25(C₃₅H₃₁N = 465.64) |
| Sub 1-63 | m/z = 605.35(C₄₃H₄₇NSi = 605.94) | Sub 1-64 | m/z = 599.3(C₄₃H₄₁NSi = 599.89) |
| Sub 1-65 | m/z = 499.26(C₃₅H₂₅D₆NSi = 499.76) | Sub 1-66 | m/z = 447.2(C₃₁H₂₉NS = 447.64) |
| Sub 1-67 | m/z = 481.19(C₃₄H₂₇NS = 481.66) | Sub 1-68 | m/z = 523.23(C₃₇H₃₃NS = 523.74) |
| Sub 1-69 | m/z = 619.23(C₄₅H₃₃NS = 619.83) | Sub 1-70 | m/z = 481.24(C₃₅H₃₁NO = 481.64) |
| Sub 1-71 | m/z = 527.22(C₃₉H₂₉NO = 527.67) | Sub 1-72 | m/z = 527.22(C₃₉H₂₉NO = 527.67) |
| Sub 1-73 | m/z = 464.65(C₃₃H₁₂D₁₃NO = 464.65) | Sub 1-74 | m/z = 601.28(C₄₆H₃₅N = 601.79) |
| Sub 1-75 | m/z = 529.19(C₃₈H₂₇NS = 529.7) | Sub 1-76 | m/z = 527.26(C₄₀H₃₃N = 527.71) |
| Sub 1-77 | m/z = 527.26(C₄₀H₃₃N = 527.71) | Sub 1-78 | m/z = 299.17(C₂₂H₂₁N = 299.42) |
| Sub 1-79 | m/z = 467.17(C₃₃H₂₅NS = 467.63) | Sub 1-80 | m/z = 709.37(C₅₄H₄₇N = 709.98) |
| Sub 1-81 | m/z = 543.2(C₃₉H₂₉NS = 543.73) | | |

### Exemplary Compounds of Sub 2

The compound belonging to Sub 2 may be, but is not limited to, a compound such as those listed below, and the FD-MS (Field Desorption-Mass Spectrometry) values of the compounds are as shown in Table 2 below.

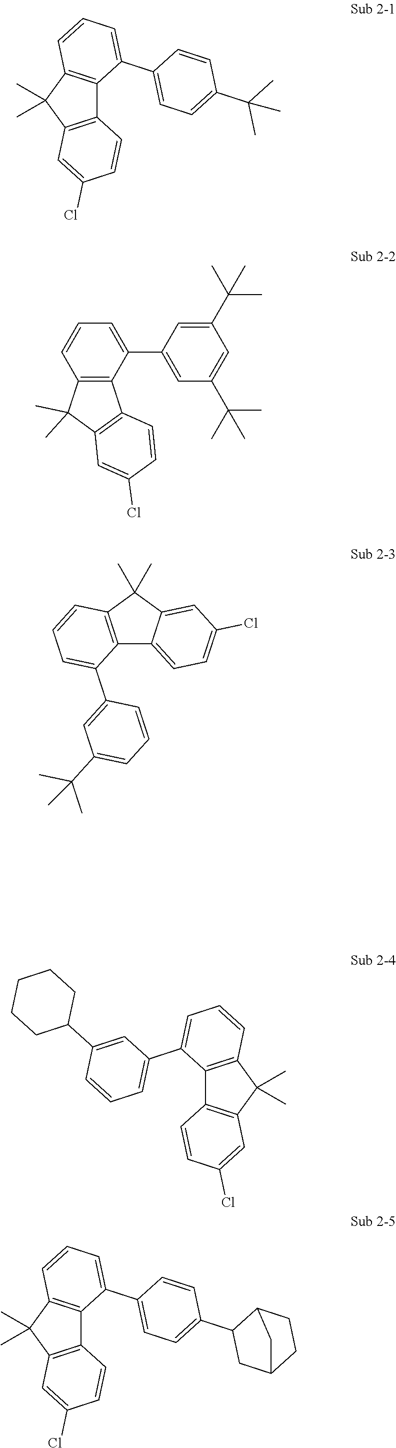

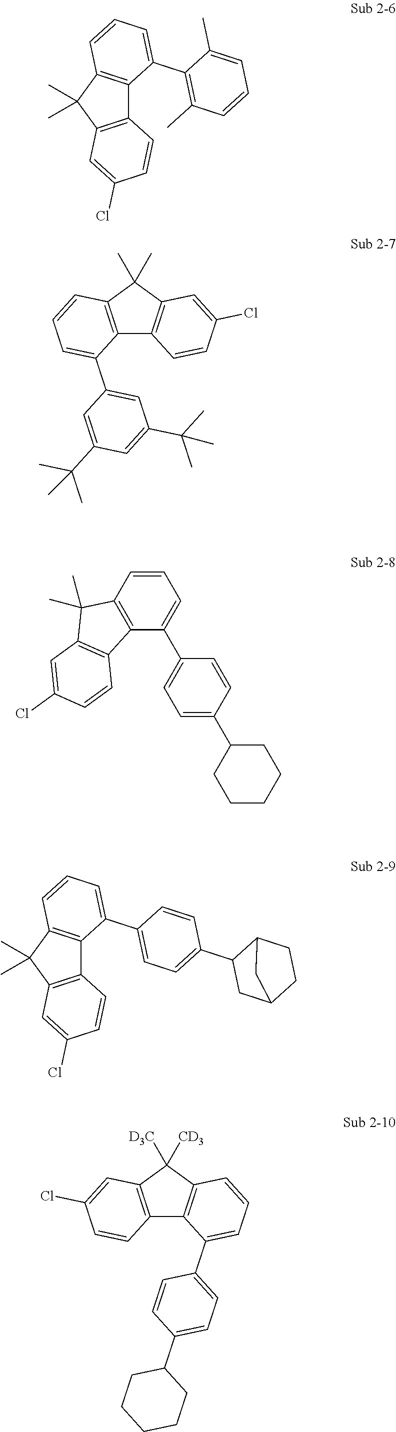

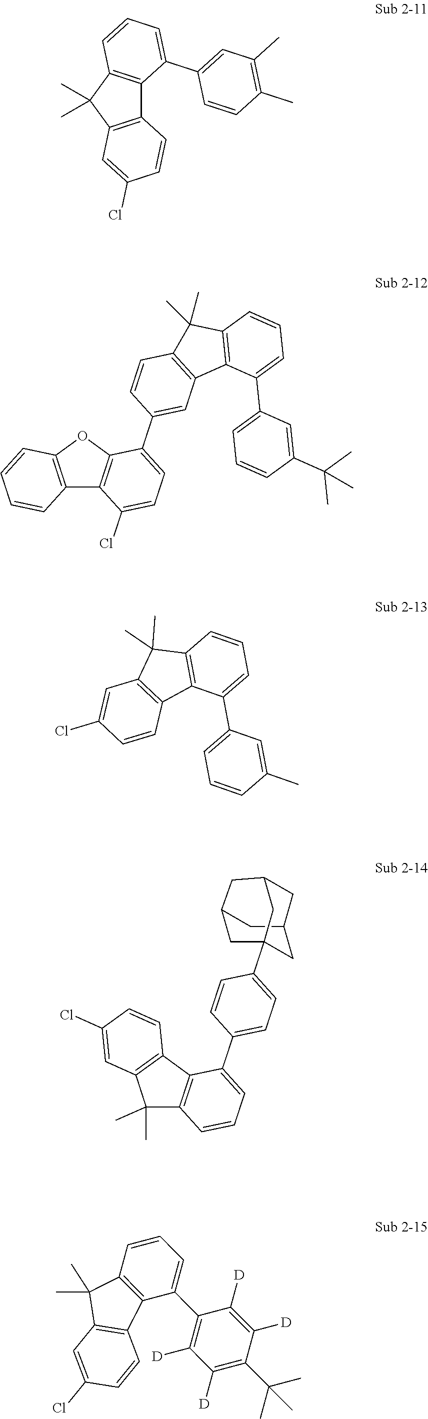

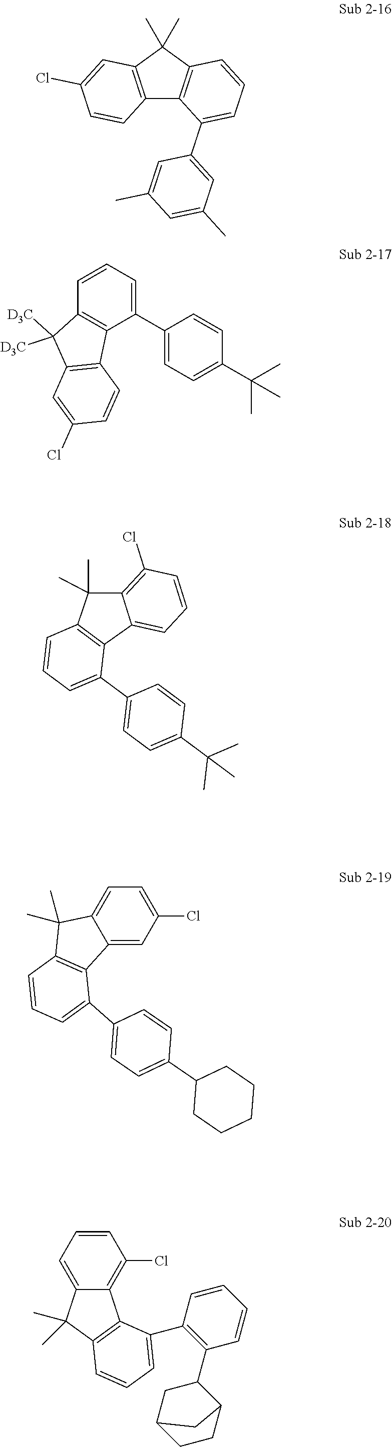

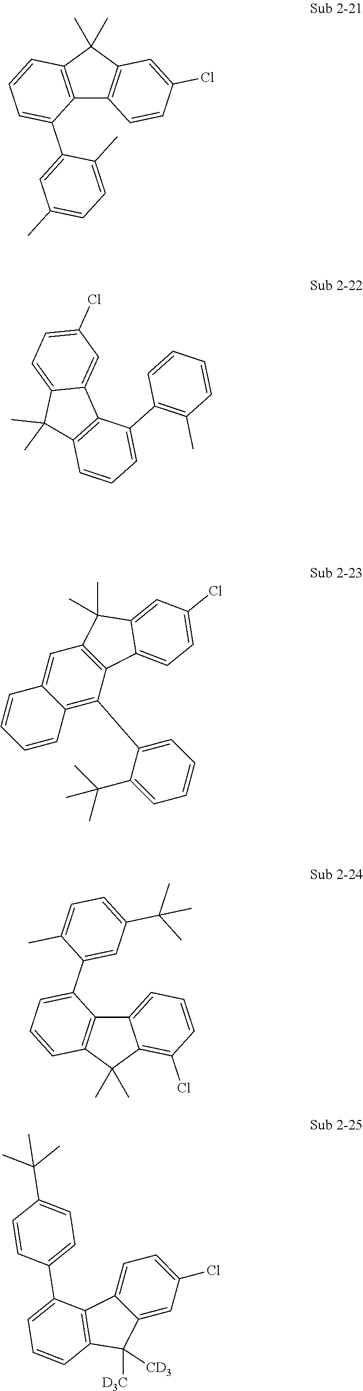 [SMILES: Cc1ccc(C)c(-c2cccc3c2-c2ccc(Cl)cc2C3(C)C)c1 ; Cc1ccccc1-c1cccc2c1-c1cc(Cl)ccc1C2(C)C ; CC(C)(C)c1ccccc1-c1c2c(cc3ccccc13)C(C)(C)c1cc(Cl)ccc1-2 ; Cc1ccc(C(C)(C)C)cc1-c1cccc2c1-c1cccc(Cl)c1C2(C)C ; [2H]C([2H])([2H])C1(C([2H])([2H])[2H])c2cc(Cl)ccc2-c2c(-c3ccc(C(C)(C)C)cc3)cccc21]

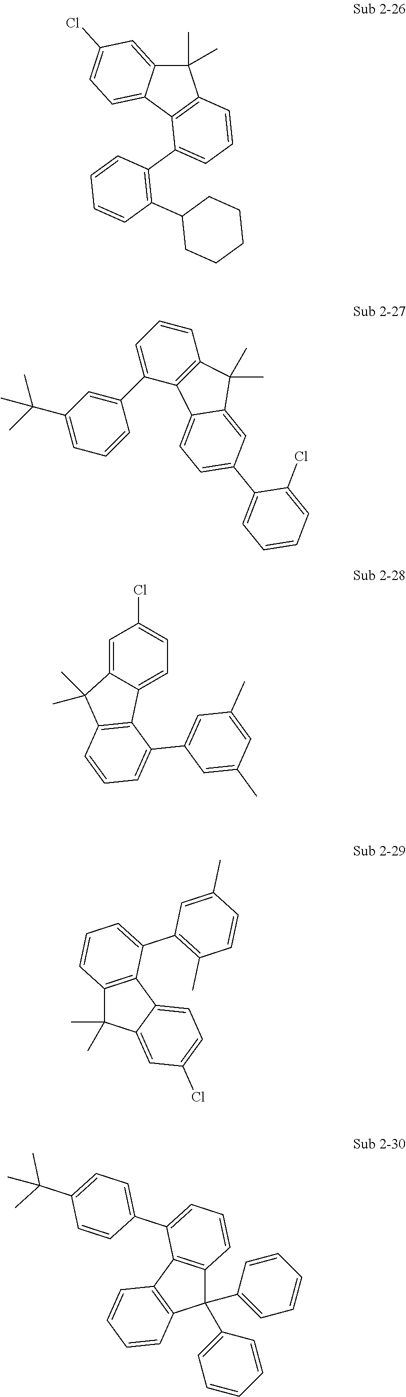

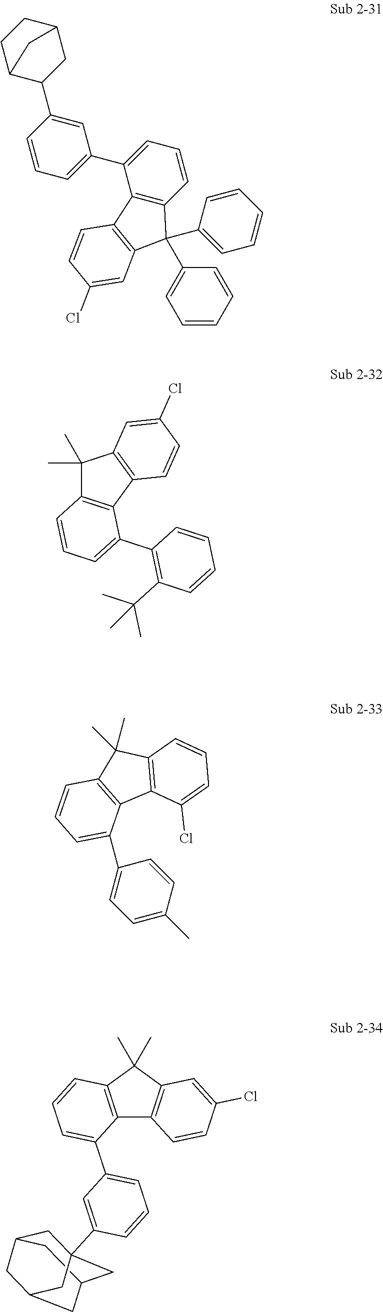 [SMILES: Clc1ccc2c(c1)C(c1ccccc1)(c1ccccc1)c1cccc(-c3cccc(C4CC5CCC4C5)c3)c1-2 ; CC(C)(C)c1ccccc1-c1cccc2c1-c1ccc(Cl)cc1C2(C)C ; Cc1ccc(-c2cccc3c2-c2c(Cl)cccc2C3(C)C)cc1 ; CC1(C)c2cc(Cl)ccc2-c2c(-c3cccc(C45CC6CC(CC(C6)C4)C5)c3)cccc21]

TABLE 2

| Compound | FD-MS | Compound | FD-MS |
|---|---|---|---|
| Sub 2-1 | m/z = 360.16(C₂₅H₂₅Cl = 360.92) | Sub 2-2 | m/z = 416.23(C₂₉H₃₃Cl = 417.03) |
| Sub 2-3 | m/z = 360.16(C₂₅H₂₅Cl = 360.92) | Sub 2-4 | m/z = 386.18(C₂₇H₂₇Cl = 386.96) |
| Sub 2-5 | m/z = 398.18(C₂₈H₂₇Cl = 398.97) | Sub 2-6 | m/z = 332.13(C₂₃H₂₁Cl = 332.87) |
| Sub 2-7 | m/z = 396.28(C₃₀H₃₆ = 396.62) | Sub 2-8 | m/z = 386.18(C₂₇H₂₇Cl = 386.96) |
| Sub 2-9 | m/z = 398.18(C₂₈H₂₇Cl = 398.97) | Sub 2-10 | m/z = 392.22(C₂₇H₂₁D₆Cl = 393) |
| Sub 2-11 | m/z = 332.13(C₂₃H₂₁Cl = 332.87) | Sub 2-12 | m/z = 526.21(C₃₇H₃₁ClO = 527.1) |
| Sub 2-13 | m/z = 318.12(C₂₂H₁₉Cl = 318.84) | Sub 2-14 | m/z = 438.21(C₃₁H₃₁Cl = 439.04) |
| Sub 2-15 | m/z = 364.19(C₂₅H₂₁D₄Cl = 364.95) | Sub 2-16 | m/z = 332.13(C₂₃H₂₁Cl = 332.87) |
| Sub 2-17 | m/z = 366.2(C₂₅H₁₉D₆Cl = 366.96) | Sub 2-18 | m/z = 360.16(C₂₅H₂₅Cl = 360.92) |
| Sub 2-19 | m/z = 386.18(C₂₇H₂₇Cl = 386.96) | Sub 2-20 | m/z = 398.18(C₂₈H₂₇Cl = 398.97) |
| Sub 2-21 | m/z = 332.13(C₂₃H₂₁Cl = 332.87) | Sub 2-22 | m/z = 318.12(C₂₂H₁₉Cl = 318.84) |
| Sub 2-23 | m/z = 410.18(C₂₉H₂₇Cl = 410.98) | Sub 2-24 | m/z = 374.18(C₂₆H₂₇Cl = 374.95) |
| Sub 2-25 | m/z = 366.2(C₂₅H₁₉D₆Cl = 366.96) | Sub 2-26 | m/z = 386.18(C₂₇H₂₇Cl = 386.96) |
| Sub 2-27 | m/z = 436.2(C₃₁H₂₉Cl = 437.02) | Sub 2-28 | m/z = 332.13(C₂₃H₂₁Cl = 332.87) |
| Sub 2-29 | m/z = 332.13(C₂₃H₂₁Cl = 332.87) | Sub 2-30 | m/z = 484.2(C₃₅H₂₉Cl = 485.07) |
| Sub 2-31 | m/z = 522.21(C₃₈H₃₁Cl = 523.12) | Sub 2-32 | m/z = 360.16(C₂₅H₂₅Cl = 360.92) |
| Sub 2-33 | m/z = 318.12(C₂₂H₁₉Cl = 318.84) | Sub 2-34 | m/z = 438.21(C₃₁H₃₁Cl = 439.04) |

### Synthesis Example of the Final Compound

### 1. Synthesis Example P-1

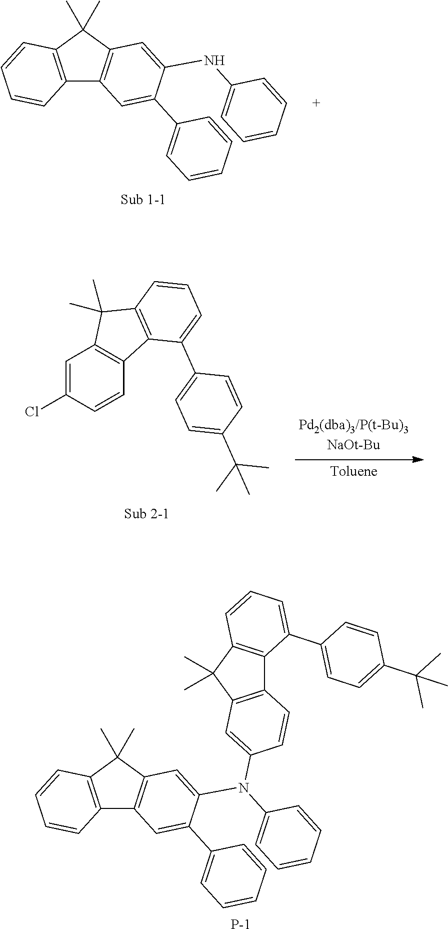

A round-bottom flask was charged with Sub 1-1 (25.0 g, 69.2 mmol), which was then dissolved in Toluene (230 mL). Subsequently, Sub 2-1 (27.5 g, 76.1 mmol), Pd₂(dba)₃ (1.9 g, 2.1 mmol), 50% P(t-Bu)₃ (1.7 mL, 4.1 mmol), and NaOt-Bu (13.3 g, 138.3 mmol) were added, and the mixture was stirred at 110° C. After the reaction was completed, the mixture was extracted with an organic solvent and water. The resulting organic layer was dried over MgSO₄ and concentrated. The concentrate was then purified by column chromatography and recrystallized to obtain the desired Product (38.9 g, Yield: 82%).

### 2. Synthesis Example P-13

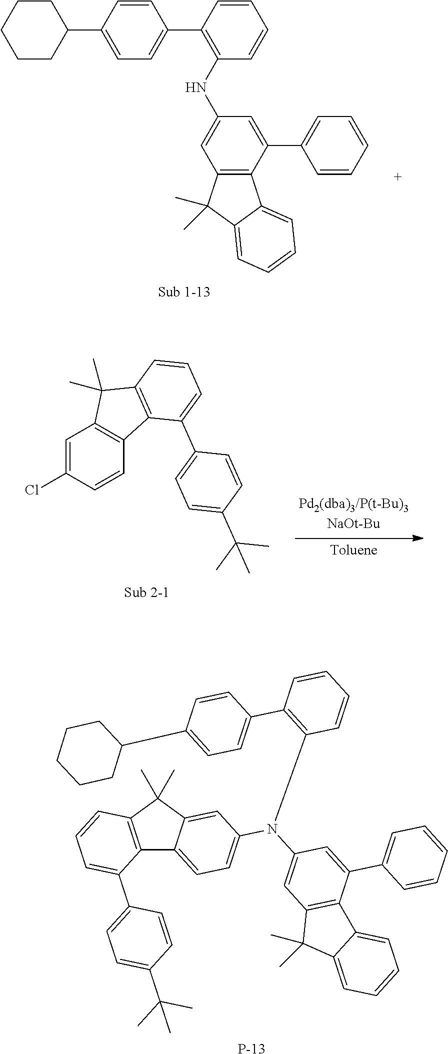

A round-bottom flask was charged with Sub 1-13 (20.0 g, 38.5 mmol), Sub 2-1 (15.3 g, 42.3 mmol), Pd₂(dba)₃ (1.1 g, 1.2 mmol), 50% P(t-Bu)₃ (0.9 mL, 2.3 mmol), and NaOt-Bu (7.4 g, 77.0 mmol). The reaction was then carried out using the same procedure as in Example of Synthesis of P-1, to obtain the desired Product (23.7 g, Yield: 73%).

### 3. Synthesis Example P-23

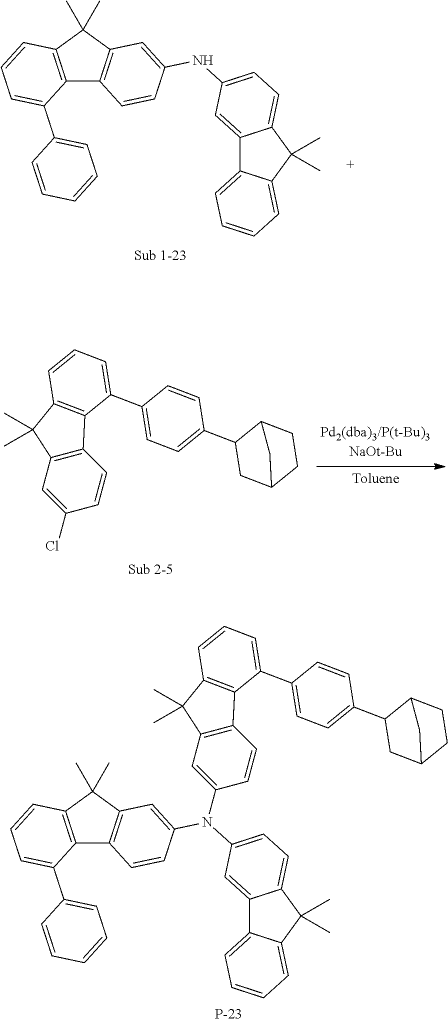

A round-bottom flask was charged with Sub 1-23 (22.0 g, 46.1 mmol), Sub 2-5 (20.2 g, 50.7 mmol), Pd₂(dba)₃ (1.3 g, 1.4 mmol), 50% P(t-Bu)₃ (1.1 mL, 2.8 mmol), and NaOt-Bu (8.9 g, 92.1 mmol). The reaction was then carried out using the same procedure as in Example of Synthesis of P-1, to obtain the desired Product (30.2 g, Yield: 78%).

### 4. Synthesis Example P-38

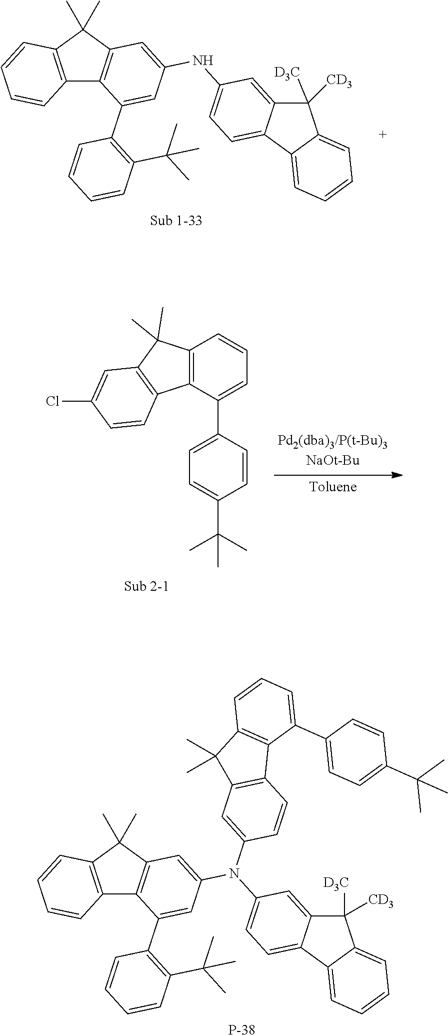

A round-bottom flask was charged with Sub 1-33 (15.0 g, 27.8 mmol), Sub 2-1 (11.0 g, 30.6 mmol), Pd₂(dba)₃ (0.8 g, 0.8 mmol), 50% P(t-Bu)₃ (0.7 mL, 1.7 mmol), and NaOt-Bu (5.3 g, 55.6 mmol). The reaction was then carried out using the same procedure as in Example of Synthesis of P-1, to obtain the desired Product (16.6 g, Yield: 69%).

### 5. Synthesis Example P-51

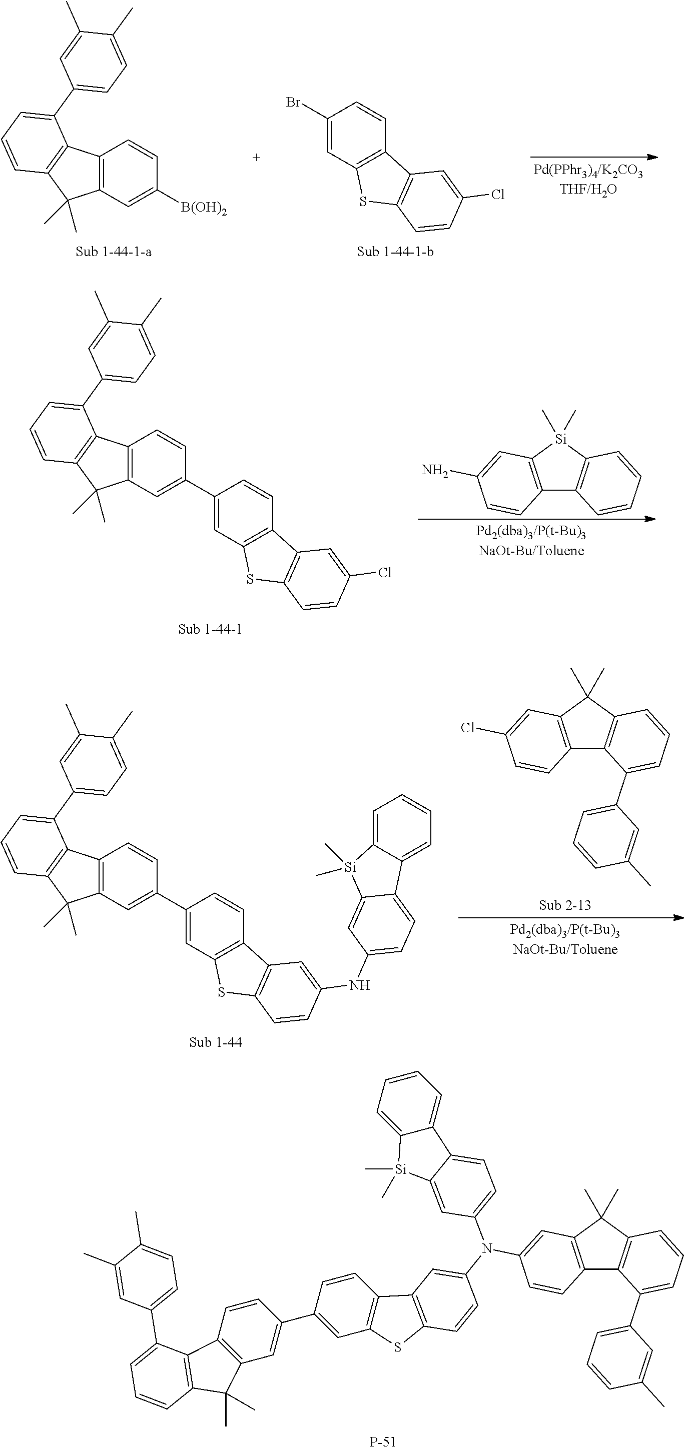

### Synthesis of Sub 1-44-1

A round-bottom flask was charged with 7-bromo-2-chlorodibenzo[b,d]thiophene (40.0 g, 134.4 mmol), which was then dissolved in THF (360 mL). Subsequently, (5-(3,4-dimethyl phenyl)-9,9-dimethyl-9H-fluoren-2-yl)boronic acid (50.6 g, 147.9 mmol), Pd(PPh₃)₄ (4.7 g, 4.0 mmol), NaOH (16.1 g, 403.2 mmol), and water (120 mL) were added, and the mixture was stirred at 75° C. After the reaction was completed, the mixture was extracted with an organic solvent and water. The resulting organic layer was dried over MgSO₄ and concentrated. The concentrate was then purified by column chromatography and recrystallized to obtain the desired Product (51.2 g, Yield: 74%).

### Synthesis of Sub 1-44

A round-bottom flask was charged with Sub 1-44-1 (30.0 g, 58.2 mmol), 5,5-dimethyl-5H-dibenzo[b,d]silol-3-amine 15.8 g, 69.9 mmol), Pd₂(dba)₃ (1.6 g, 1.7 mmol), 50% P(t-Bu)₃ (1.4 ml, 3.5 mmol), and NaOt-Bu (11.2 g, 116.5 mmol). The reaction was then carried out using the same procedure as in Example of Synthesis of P-1, to obtain the desired Product (29.5 g, Yield: 72%).

### Synthesis of P-51

A round-bottom flask was charged with Sub 1-16 (14.0 g, 19.9 mmol), Sub 2-13 (7.6 g, 23.9 mmol), Pd₂(dba)₃ (0.5 g, 0.6 mmol), 50% P(t-Bu)₃ (0.5 ml, 1.2 mmol), and NaOt-Bu (3.8 g, 39.8 mmol). The reaction was then carried out using the same procedure as in Example of Synthesis of P-1, to obtain the desired Product (12.0 g, Yield: 61%).

### 6. Synthesis Example P-58

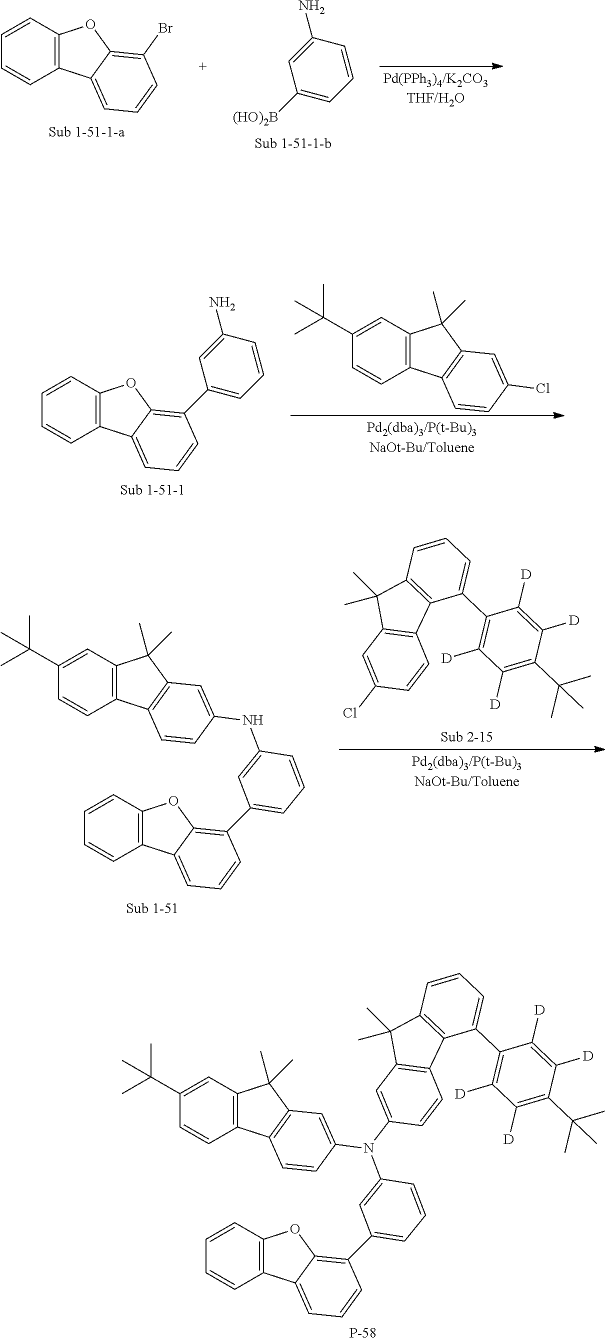

### Synthesis of Sub 1-51-1

A round-bottom flask was charged with Sub 1-51-1-a (35.0 g, 141.6 mmol), Sub 1-51-1-b (23.3 g, 170.0 mmol), Pd(PPh₃)₄ (4.9 g, 4.2 mmol), and NaOH (17.0 g, 424.9 mmol). The reaction was then carried out using the same procedure as in Example of Synthesis of Sub 1-44-1, to obtain the desired Product (21.7 g, Yield: 59%).

### Synthesis of Sub 1-51

A round-bottom flask was charged with Sub 1-44-1 (21.7 g, 76.2 mmol), 2-(tert-butyl)-7-chloro-9,9-dimethyl-9H-fluorene (19.8 g, 76.2 mmol), Pd₂(dba)₃ (2.1 g, 2.3 mmol), 50% P(t-Bu)₃ (1.8 ml, 4.6 mmol), and NaOt-Bu (14.6 g, 152.4 mmol). The reaction was then carried out using the same procedure as in Example of Synthesis of P-1, to obtain the desired Product (24.8 g, Yield: 64%).

### Synthesis of P-58

A round-bottom flask was charged with Sub 1-51 (15.0 g, 29.5 mmol), Sub 2-15 (11.9 g, 32.5 mmol), Pd₂(dba)₃ (0.8 g, 0.9 mmol), 50% P(t-Bu)₃ (0.7 ml, 1.8 mmol), and NaOt-Bu (5.7 g, 59.1 mmol). The reaction was then carried out using the same procedure as in Example of Synthesis of P-1, to obtain the desired Product (18.0 g, Yield: 73%).

### 7. Synthesis Example P-71

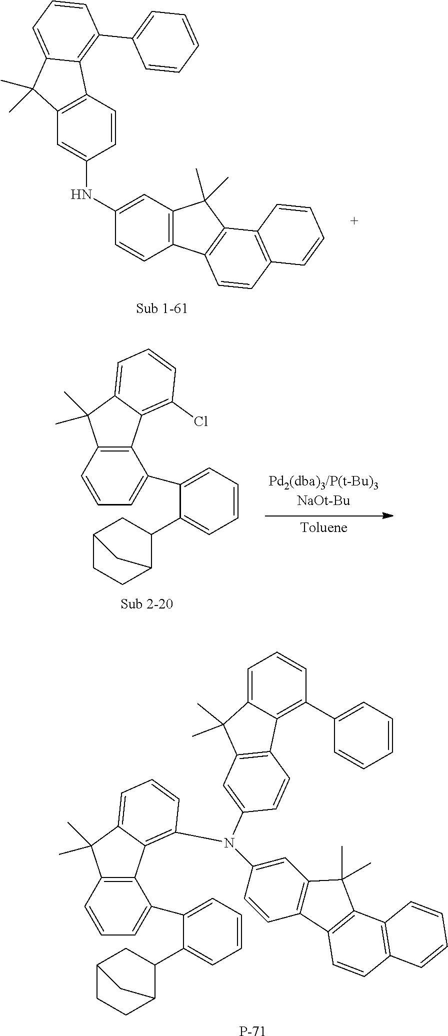

A round-bottom flask was charged with Sub 1-61 (20.0 g, 37.9 mmol), Sub 2-20 (22.7 g, 56.8 mmol), Pd₂(dba)₃ (1.0 g, 1.1 mmol), 50% P(t-Bu)₃ (0.9 mL, 2.3 mmol), and NaOt-Bu (7.3 g, 15.7 mmol). The reaction was then carried out using the same procedure as in Example of Synthesis of P-1, to obtain the desired Product (13.5 g, Yield: 40%).

### 8. Synthesis Example P-79

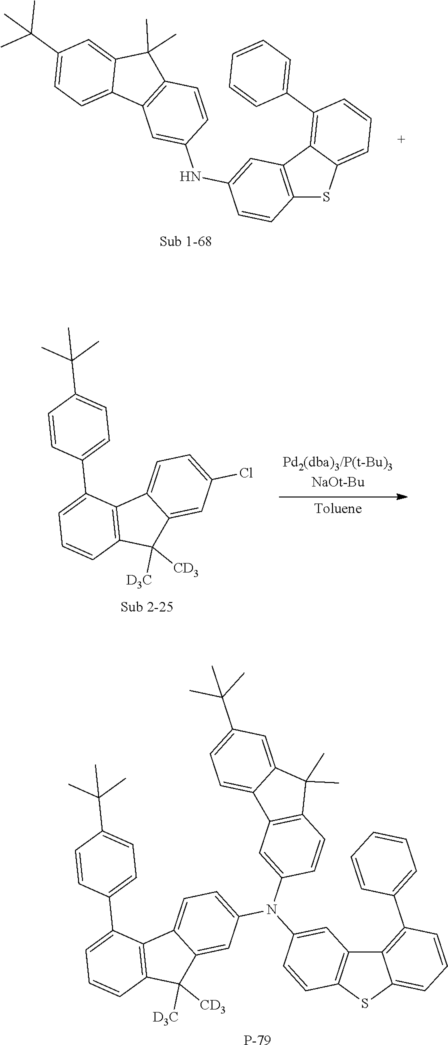

A round-bottom flask was charged with Sub 1-68 (15.0 g, 28.6 mmol), Sub 2-25 (11.6 g, 31.5 mmol), Pd₂(dba)₃ (0.8 g, 0.9 mmol), 50% P(t-Bu)₃ (0.7 mL, 1.7 mmol), and NaOt-Bu (5.5 g, 57.3 mmol). The reaction was then carried out using the same procedure as in Example of Synthesis of P-1, to obtain the desired Product (19.1 g, Yield: 78%).

### 9. Synthesis Example P-85

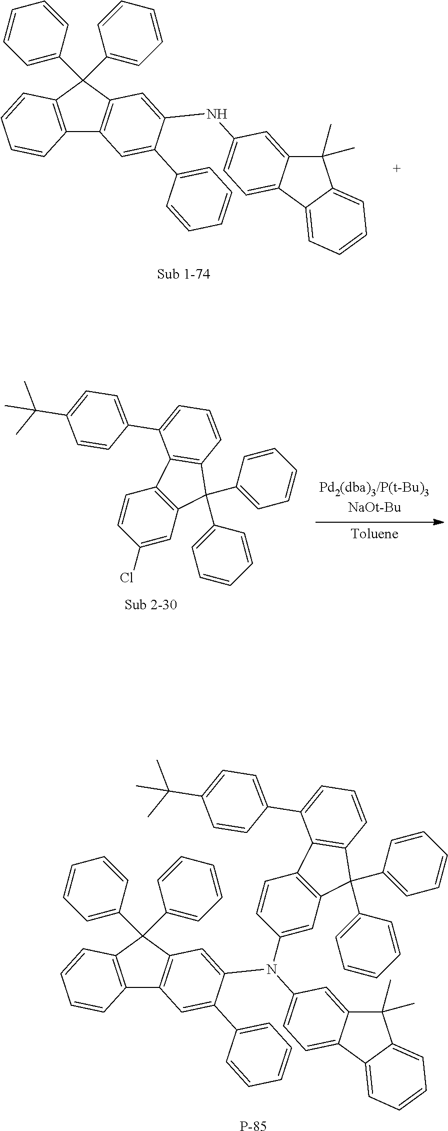

A round-bottom flask was charged with Sub 1-74 (10.0 g, 16.6 mmol), Sub 2-30 (8.9 g, 18.3 mmol), Pd₂(dba)₃ (0.5 g, 0.5 mmol), 50% P(t-Bu)₃ (0.4 mL, 1.0 mmol), and NaOt-Bu (3.2 g, 33.2 mmol). The reaction was then carried out using the same procedure as in Example of Synthesis of P-1, to obtain the desired Product (12.7 g, Yield: 73%).

The FD-MS values of the compounds P-1 to P-92 of the present invention prepared according to the synthesis examples described above are shown in Table 3 below.

TABLE 3

| Compound | FD-MS | Compound | FD-MS |
|---|---|---|---|
| P-1 | m/z = 685.37(C₅₂H₄₇N = 685.96) | P-2 | m/z = 761.4(C₅₈H₅₁N = 762.05) |
| P-3 | m/z = 761.4(C₅₈H₅₁N = 762.05) | P-4 | m/z = 761.4(C₅₈H₅₁N = 762.05) |
| P-5 | m/z = 811.42(C₆₂H₅₃N = 812.11) | P-6 | m/z = 837.43(C₆₄H₅₅N = 838.15) |
| P-7 | m/z = 837.43(C₆₄H₅₅N = 838.15) | P-8 | m/z = 837.43(C₆₄H₅₅N = 838.15) |
| P-9 | m/z = 893.5(C₆₈H₆₃N = 894.26) | P-10 | m/z = 813.53(C₆₁H₆₇N = 814.21) |
| P-11 | m/z = 817.46(C₆₂H₅₉N = 818.16) | P-12 | m/z = 817.46(C₆₂H₅₉N = 818.16) |
| P-13 | m/z = 843.48(C₆₄H₆₁N = 844.2) | P-14 | m/z = 819.48(C₆₂H₆₁N = 820.18) |
| P-15 | m/z = 779.45(C₅₉H₅₇N = 780.11) | P-16 | m/z = 793.46(C₆₀H₅₉N = 794.14) |
| P-17 | m/z = 875.45(C₆₇H₅₇N = 876.2) | P-18 | m/z = 809.4(C₆₂H₅₁N = 810.1) |
| P-19 | m/z = 887.45(C₆₈H₅₇N = 888.21) | P-20 | m/z = 837.43(C₆₄H₅₅N = 838.15) |
| P-21 | m/z = 801.43(C₆₁H₅₅N = 802.12) | P-22 | m/z = 827.45(C₆₃H₅₇N = 828.16) |
| P-23 | m/z = 839.45(C₆₄H₅₇N = 840.17) | P-24 | m/z = 773.4(C₅₉H₅₁N = 774.06) |
| P-25 | m/z = 801.43(C₆₁H₅₅N = 802.12) | P-26 | m/z = 801.43(C₆₁H₅₅N = 802.12) |
| P-27 | m/z = 801.43(C₆₁H₅₅N = 802.12) | P-28 | m/z = 801.43(C₆₁H₅₅N = 802.12) |
| P-29 | m/z = 953.46(C₇₂H₅₉NO = 954.27) | P-30 | m/z = 925.46(C₇₁H₅₉N = 926.26) |
| P-31 | m/z = 857.5(C₆₅H₆₃N = 858.23) | P-32 | m/z = 815.45(C₆₂H₅₇N = 816.14) |
| P-33 | m/z = 833.49(C₆₃H₅₁D₆N = 834.19) | P-34 | m/z = 833.49(C₆₃H₅₁D₆N = 834.19) |
| P-35 | m/z = 895.5(C₆₈H₅₃D₆N = 896.26) | P-36 | m/z = 921.59(C₆₉H₄₃D₁₈N = 922.36) |
| P-37 | m/z = 877.46(C₆₇H₅₉N = 878.22) | P-38 | m/z = 863.53(C₆₅H₅₇D₆N = 864.26) |
| P-39 | m/z = 819.55(C₆₁H₃₇D₁₈N = 820.23) | P-40 | m/z = 883.51(C₆₇H₆₅N = 884.26) |
| P-41 | m/z = 925.46(C₇₁H₅₉N = 926.26) | P-42 | m/z = 925.46(C₇₁H₅₉N = 926.26) |
| P-43 | m/z = 902.46(C₆₉H₅₀D₅N = 903.24) | P-44 | m/z = 857.5(C₆₅H₆₃N = 858.23) |
| P-45 | m/z = 817.41(C₆₀H₅₅NSi = 818.19) | P-46 | m/z = 843.43(C₆₂H₅₇NSi = 844.23) |
| P-47 | m/z = 843.43(C₆₂H₅₇NSi = 844.23) | P-48 | m/z = 902.58(C₆₅H₅₀D₁₅NSi = 903.42) |
| P-49 | m/z = 973.5(C₇₂H₅₉D₄NSi = 974.41) | P-50 | m/z = 983.45(C₇₂H₆₁NOSi = 984.37) |
| P-51 | m/z = 985.41(C₇₁H₅₉NSSi = 986.41) | P-52 | m/z = 999.52(C₇₄H₆₉NSi = 1000.46) |
| P-53 | m/z = 775.38(C₅₈H₄₉NO = 776.04) | P-54 | m/z = 801.4(C₆₀H₅₁NO = 802.07) |
| P-55 | m/z = 813.4(C₆₁H₅₁NO = 814.08) | P-56 | m/z = 801.4(C₆₀H₅₁NO = 802.07) |
| P-57 | m/z = 815.41(C₆₁H₅₃NO = 816.1) | P-58 | m/z = 835.47(C₆₂H₅₃D₄NO = 836.17) |
| P-59 | m/z = 851.41(C₆₄H₅₃NO = 852.13) | P-60 | m/z = 761.37(C₅₇H₄₇NO = 762.01) |
| P-61 | m/z = 829.37(C₆₁H₅₁NS = 830.15) | P-62 | m/z = 817.37(C₆₀H₅₁NS = 818.14) |
| P-63 | m/z = 791.36(C₅₈H₄₉NS = 792.1) | P-64 | m/z = 829.37(C₆₁H₅₁NS = 830.15) |
| P-65 | m/z = 581.31(C₄₄H₃₉N = 581.8) | P-66 | m/z = 791.36(C₅₈H₄₉NS = 792.1) |
| P-67 | m/z = 1033.56(C₇₉H₇₁N = 1034.44) | P-68 | m/z = 945.44(C₇₀H₅₉NS = 946.31) |
| P-69 | m/z = 817.46(C₆₂H₅₉N = 818.16) | P-70 | m/z = 843.48(C₆₄H₆₁N = 844.2) |
| P-71 | m/z = 889.46(C₆₈H₅₉N = 890.23) | P-72 | m/z = 839.45(C₆₄H₅₇N = 840.17) |
| P-73 | m/z = 843.43(C₆₂H₅₇NSi = 844.23) | P-74 | m/z = 901.5(C₆₆H₆₇NSi = 902.35) |
| P-75 | m/z = 867.43(C₆₄H₅₇NSi = 868.25) | P-76 | m/z = 823.45(C₆₀H₄₉D₆NSi = 824.23) |
| P-77 | m/z = 785.41(C₅₇H₅₅NS = 786.13) | P-78 | m/z = 805.37(C₅₉H₅₁NS = 806.12) |
| P-79 | m/z = 853.46(C₆₂H₅₁D₆NS = 854.24) | P-80 | m/z = 969.44(C₇₂H₅₉NS = 970.33) |
| P-81 | m/z = 881.46(C₆₆H₅₉NO = 882.2) | P-82 | m/z = 877.43(C₆₆H₅₅NO = 878.17) |
| P-83 | m/z = 823.38(C₆₂H₄₉NO = 824.08) | P-84 | m/z = 760.43(C₅₆H₃₂D₁₃NO = 761.06) |
| P-85 | m/z = 1049.5(C₈₁H₆₃N = 1050.4) | P-86 | m/z = 1015.42(C₇₆H₅₇NS = 1016.36) |
| P-87 | m/z = 851.45(C₆₅H₅₇N = 852.18) | P-88 | m/z = 851.45(C₆₅H₅₇N = 852.18) |
| P-89 | m/z = 851.45(C₆₅H₅₇N = 852.18) | P-90 | m/z = 941.44(C₇₀H₅₉NSi = 942.33) |
| P-91 | m/z = 941.44(C₇₀H₅₉NSi = 942.33) | P-92 | m/z = 817.41(C₆₀H₅₅NSi = 818.19) |

Although the above description provides synthesis examples of the compounds represented by Formula 1, these are all based on reactions such as the Buchwald-Hartwig cross-coupling reaction, Miyaura boration reaction, Suzuki cross-coupling reaction, intramolecular acid-induced cyclization (J. Mater. Chem. 1999, 9, 2095), Pd(II)-catalyzed oxidative cyclization (Org. Lett. 2011, 13, 5504), and PPh₃-mediated reductive cyclization (J. Org. Chem. 2005, 70, 5014). It will be readily understood by those skilled in the art that these reactions may proceed even when substituents other than those specifically described in the synthesis examples are introduced, as long as they fall within the scope defined for Formula 1.

### Manufacturing and Evaluation of Organic Electronic Device

### [Test Example 1] Red Organic Electroluminescent Device (Light-Emitting Auxiliary Layer)

A hole injection layer with a thickness of 10 nm is formed by vacuum-depositing N-([1,1′-biphenyl]-4-yl)-9,9-dimethyl-N-(4-(9-phenyl-9H-carbazol-3-yl)phenyl)-9H-fluoren-2-amine (hereinafter referred to as Compound A) and 4,4′,4″-((1E,1′E,1″E)-cyclopropane-1,2,3-triylidenetris(cyanomethaneylylidene))tris(2,3,5,6-tetrafluorobenzonitrile) (hereinafter referred to as Compound B) on an ITO layer (anode) formed on a glass substrate. At this time, Compound B is doped so that the weight ratio of Compound A to Compound B is 98:2.

Next, a hole transport layer with a thickness of 110 nm is formed by vacuum-depositing Compound A on the hole injection layer.

Subsequently, a light-emitting auxiliary layer with a thickness of 10 nm is formed by vacuum-depositing the Compound P-1 of the present invention on the hole transport layer.

Next, a light-emitting layer with a thickness of 30 nm is formed by vacuum-depositing host and dopant on the light-emitting auxiliary layer, wherein the host is 14-(4-phenylquinazolin-2-yl)-14H-benzo[c]benzo[4,5]thieno[2,3-a]carbazole, and the dopant is bis-(1-phenylisoquinolyl)iridium(III) acetylacetonate (hereinafter abbreviated as ‘(piq)₂Ir(acac)’). At this time, the dopant is doped such that the weight ratio of the host to the dopant is 95:5.

Next, a hole blocking layer with a thickness of 10 nm is formed by vacuum-depositing 2-(4′-(9,9-dimethyl-9H-fluoren-2-yl)-[1,1′-biphenyl]-3-yl)-4,6-diphenyl-1,3,5-triazine on the light-emitting layer.

Next, a hole blocking layer with a thickness of 10 nm is formed by vacuum-depositing 2-(4′-(9,9-dimethyl-9H-fluoren-2-yl)-[1,1′-biphenyl]-3-yl)-4,6-diphenyl-1,3,5-triazine on the light-emitting layer.

Subsequently, an electron transport layer with a thickness of 30 nm is formed by vacuum-depositing a mixture of 2,7-bis(4-(4,6-diphenyl-1,3,5-triazin-2-yl)phenyl)naphthalene and (8-quinolinolato)lithium in a 5:5 weight ratio on the hole blocking layer.

Then, an electron injection layer with a thickness of 0.2 nm is formed by vacuum-depositing (8-quinolinolato)lithium on the electron transport layer, followed by deposition of Al to form a cathode with a thickness of 150 nm.

### [Test Examples 2] to [Test Example 19]

Organic electroluminescent devices were fabricated in the same manner as in Example 1, except that the compounds of the present invention listed in Table 5 below were used as a light-emitting auxiliary layer material instead of the Compound P-1 of the present invention.

### [Comparative Examples 1] to [Comparative Examples 3]

Organic electroluminescent devices were fabricated in the same manner as in Example 1, except that one of the Comparative Compounds A to C below was used as a light-emitting auxiliary layer material instead of the Compound P-1 of the present invention.

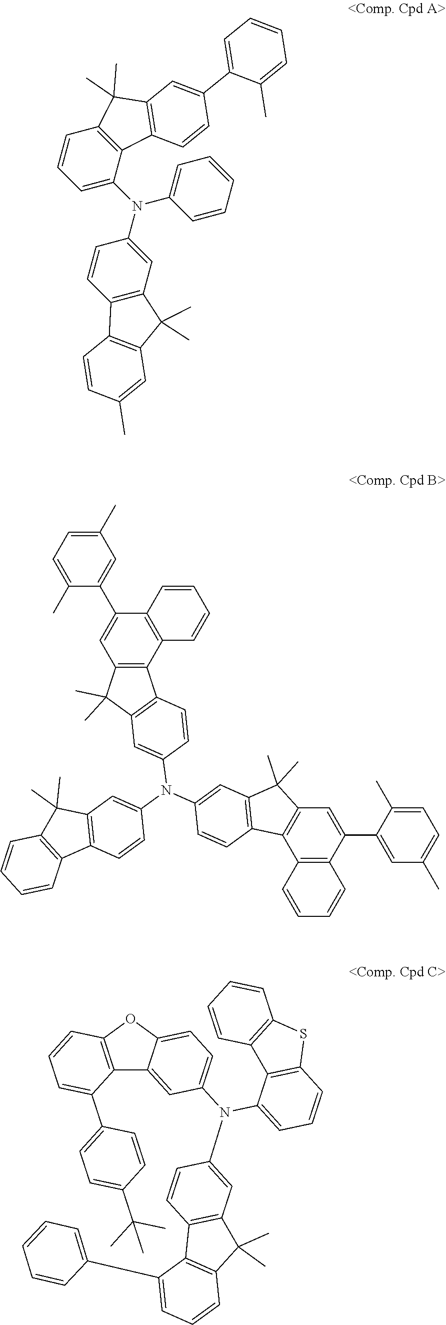 [SMILES: Cc1ccc2c(c1)C(C)(C)c1cc(N(c3ccccc3)c3cccc4c3-c3ccc(-c5ccccc5C)cc3C4(C)C)ccc1-2 ; Cc1ccc(C)c(-c2cc3c(c4ccccc24)-c2ccc(N(c4ccc5c(c4)C(C)(C)c4ccccc4-5)c4ccc5c(c4)C(C)(C)c4cc(-c6cc(C)ccc6C)c6ccccc6c4-5)cc2C3(C)C)c1 ; CC(C)(C)c1ccc(-c2cccc3oc4ccc(N(c5ccc6c(c5)C(C)(C)c5cccc(-c7ccccc7)c5-6)c5cccc6sc7ccccc7c56)cc4c23)cc1]

The electroluminescent (EL) characteristics of the organic electroluminescent devices fabricated according to the test Examples 1 to 19, and Comparative Examples 1 to 3 were measured by applying a forward DC voltage using a PR-650 photometer from Photo Research. The T₉₅ lifetime was measured at a standard luminance of 2500 cd/m² using a lifetime measurement system manufactured by Mc Science. The measurement results are shown in Table 4 below.

The measuring device is independent from possible day-to-day variations of deposition rates, vacuum quality or other tool performance parameters, and allows assessing performance of new material in comparison with comparative compound under the same conditions. At the time of assessment, each batch contained 4 identically prepared OLEDs including a comparative compound, and since the performance of each of a total of 12 OLEDs in 3 batchs is evaluated, the statistical evaluation of the obtained experimental results unequivocally showed the statistical significance.

TABLE 4

| | | | Current | Bright- | Effi- | |
|---|---|---|---|---|---|---|
| | | Voltage | Density | ness | ciency | |
| | Compound | (V) | (mA/cm²) | (cd/m²) | (cd/A) | T(95) |
| Comp. Ex | Comp. | 5.1 | 13.5 | 2500.0 | 18.5 | 94.9 |
| 1 | compd A | | | | | |
| Comp. Ex | Comp. | 5.3 | 14.5 | 2500.0 | 17.2 | 86.1 |
| 2 | compd B | | | | | |
| Comp. Ex | Comp. | 5.1 | 12.8 | 2500.0 | 19.6 | 91.1 |
| 3 | compd C | | | | | |
| Test Ex. | P-1 | 4.1 | 7.1 | 2500.0 | 35.2 | 120.1 |
| 1 | | | | | | |
| Test Ex. | P-9 | 4.1 | 7.2 | 2500.0 | 34.9 | 123.5 |
| 2 | | | | | | |
| Test Ex. | P-14 | 4.1 | 6.9 | 2500.0 | 36.1 | 121.1 |
| 3 | | | | | | |
| Test Ex. | P-15 | 4.1 | 7.2 | 2500.0 | 34.9 | 125.6 |
| 4 | | | | | | |
| Test Ex. | P-38 | 4.1 | 6.3 | 2500.0 | 39.6 | 129.5 |
| 5 | | | | | | |
| Test Ex. | P-46 | 4.5 | 7.1 | 2500.0 | 35.4 | 115.5 |
| 6 | | | | | | |
| Test Ex. | P-53 | 4.4 | 6.6 | 2500.0 | 37.7 | 114.0 |
| 7 | | | | | | |
| Test Ex. | P-56 | 4.5 | 7.1 | 2500.0 | 35.0 | 111.8 |
| 8 | | | | | | |
| Test Ex. | P-60 | 4.5 | 6.7 | 2500.0 | 37.2 | 113.5 |
| 9 | | | | | | |
| Test Ex. | P-63 | 4.5 | 7.0 | 2500.0 | 35.8 | 117.8 |
| 10 | | | | | | |
| Test Ex. | P-65 | 4.2 | 6.5 | 2500.0 | 38.5 | 120.9 |
| 11 | | | | | | |
| Test Ex. | P-66 | 4.3 | 6.8 | 2500.0 | 36.5 | 125.0 |
| 12 | | | | | | |
| Test Ex. | P-67 | 4.1 | 8.1 | 2500.0 | 30.7 | 111.6 |
| 13 | | | | | | |
| Test Ex. | P-69 | 4.2 | 6.9 | 2500.0 | 36.0 | 122.9 |
| 14 | | | | | | |
| Test Ex. | P-74 | 4.5 | 6.9 | 2500.0 | 36.3 | 120.3 |
| 15 | | | | | | |
| Test Ex. | P-79 | 4.3 | 6.8 | 2500.0 | 36.6 | 124.4 |
| 16 | | | | | | |
| Test Ex. | P-80 | 4.5 | 6.8 | 2500.0 | 36.8 | 120.9 |
| 17 | | | | | | |
| Test Ex. | P-85 | 4.2 | 7.0 | 2500.0 | 35.9 | 127.5 |
| 18 | | | | | | |
| Test Ex. | P-91 | 4.4 | 7.1 | 2500.0 | 35.4 | 120.6 |
| 19 | | | | | | |

As can be seen from the results in Table 4 above, when the compound of the present invention is used as the light-emitting auxiliary layer material, the driving voltage, luminous efficiency, and lifetime of the organic electroluminescent device can be improved compared to Comparative Examples 1 to 3 which use one of Comparative Compound A to Comparative Compound C, wherein the comparative compounds have a similar basic skeleton to the compound of the present invention.

The present invention is a tertiary amine structure which is substituted with a fluorenyl group represented by Formula A and a fluorenyl group represented by Formula B. In Formula B, the 5-position of the fluorenyl group is substituted with a phenyl group, and this phenyl group is substituted with Ak, which is an alkyl group or an alicyclic group.

First, examining Comparative Compound A and the compound of the present invention, Comparative Compound A is similar to the compound of the present invention in that it is a tertiary amine compound substituted with two fluorenyl groups, but it differs from the present invention in that the position of the phenyl group substituted on the fluorenyl group is the 7-position.

To confirm the change in the compound's energy level due to this difference, the HOMO value was measured using the DFT method (B3LYP/6-31g(D)) of the Gaussian program. The measured values are as shown in Table 5 below.

TABLE 5

| Compound | P-65 | Comp. compd A |
|---|---|---|
| HOMO(eV) | −4.74 | −4.80 |

As can be seen from the results in Table 5 above, the HOMO Energy Level of Compound P-65 of the present invention, which is structurally most similar to Comparative Compound A, is higher than that of Comparative Compound A.

Therefore, since the HOMO value of Comparative Compound A is deeper (lower), the hole injection from the light-emitting auxiliary layer to the Emitting Layer becomes excessive, causing holes to accumulate within the Emitting Layer, and consequently, the lifetime and efficiency of the device appear to decrease.

On the other hand, when Compound P-65 of the present invention, which is structurally similar to Comparative Compound A, is used as the light-emitting auxiliary layer material, holes are injected appropriately from the light-emitting auxiliary layer to the Emitting Layer. This appears to achieve charge balance in the device, thereby improving the efficiency and lifetime.

Next, Comparative Compound B is also similar to the compound of the present invention in that it is a tertiary amine compound containing two fluorene moieties, but it differs from the present invention in the presence or absence of a substituent at the 5-position of the fluorene. That is, in the present invention, the 5-position of the fluorene is substituted with a phenyl group substituted with Ak, whereas Comparative Compound B does not include such a fluorene moiety.

Furthermore, Comparative Compound C differs from the compound of the present invention in that it has only one fluorene moiety in its molecule.

Due to these differences, Comparative Compound B and Comparative Compound C have a relatively higher refractive index compared to the compound of the present invention, and consequently, the efficiency of the device appears to differ significantly.

As can be seen through the results of Table 4 and Table 5 above, even compounds having similar configurations, the compound of the present invention-which satisfies complex factors such as the type of a specific substituent and the substitution position of the substituent-shows a remarkable effect in an organic electronic device compared to other comparative compounds. Through this, it can be understood that the compound of the present invention will exhibit a remarkable effect in an organic electronic device compared to simple structural isomers or compounds having similar configurations that are not described herein.

These results suggest that, even among compounds having similar molecular components, the properties of the compound-such as hole characteristics, luminous efficiency characteristics, energy level, hole injection and mobility characteristics, charge balance of holes and electrons, volume density, and intermolecular distance—can be significantly and unpredictably changed depending on the type of substituent and the position of substitution. Furthermore, it suggests that the performance of the device can change not just due to a single component, but due to complex factors.

In the case of a light-emitting auxiliary layer, the interrelationship between a hole transport layer and a light-emitting layer (host) must be understood. It would be very difficult, even for a person skilled in the art, to infer the characteristics exhibited by a light-emitting auxiliary layer using the compound of the present invention, even when using compounds having similar core structure.

Furthermore, although the evaluation results of the previously described device fabrication explained the device characteristics when the compound of the present invention was applied only to a light-emitting auxiliary layer, the compound of the present invention can also be used by applying it to a hole transport layer or by applying it to both a hole transport layer and a light-emitting auxiliary layer.

The foregoing description is merely illustrative of the present invention, and it will be possible for one of ordinary skill in the art to make various modifications without departing from the essential characteristics of the present invention. The scope of protection of the present invention should be construed by the accompanying claims, and all equivalents falling within the scope thereof should be construed as being included in the scope of the rights of the present invention.

Description of Reference Numerals

| | |
|---|---|
| 100, 200, 300: organic electronic | 110: first electrode |
| device | |
| 120: hole injection layer | 130: hole transport layer |
| 140: light-emitting layer | 150: electron transport layer |
| 160: electron injection layer | 170: second electrode |
| 180: layer for improving light | 210: buffer layer |
| efficiency | |
| 220: light-emitting auxiliary layer | 320: first hole injection layer |
| 330: first hole transport layer | 340: first light-emitting layer |
| 350: first electron transport layer | 360: first charge-generation layer |
| 361: second charge-generation layer | 420: second hole injection layer |
| 430: second hole transport layer | 440: second light-emitting layer |
| 450: second electron transport layer | CGL: charge generation layer |
| ST1: first stack | ST2: second stack |

1. A compound of Formula 1: 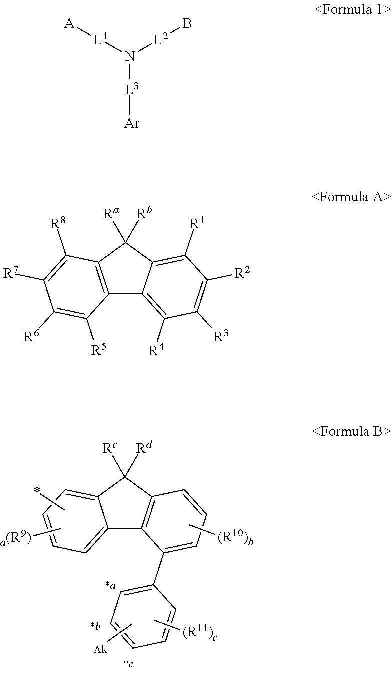
in Formula 1,
A is Formula A and is bonded to L¹ through one of R¹ to R⁸,
B is Formula B and is bonded to L² through the asterisk (*),
Ar is selected from the group consisting of a C₆-C₆₀ aryl group, a fluorenyl group, C₂-C₆₀ heterocyclic group containing at least one heteroatom of O, N, S, Si and P, a C₃-C₆₀ alicyclic group, and a fused ring of a C₆-C₆₀ aromatic ring and a C₃-C₆₀ alicyclic group,
L¹ to L³ are each independently selected from the group consisting of a sing bond, a C₆-C₆₀ arylene group, a fluorenylene group, a C₂-C₆₀ heterocyclic group containing at least one heteroatom of O, N, S, Si and P, a C₃-C₆₀ alicyclic group, and a fused ring of a C₆-C₆₀ aromatic ring and a C₃-C₆₀ alicyclic group,
R¹ to R¹¹, R^{a} to R^{d} are each independently selected from the group consisting of hydrogen, deuterium, halogen, a cyano group, a C₆-C₆₀ aryl group, a fluorenyl group, a C₂-C₆₀ heterocyclic group containing at least one heteroatom of O, N, S, Si and P, a C₃-C₆₀ alicyclic group, a fused ring of a C₆-C₆₀ aromatic ring and a C₃-C₆₀ alicyclic group, a C₁-C₂₀ alkyl group, a C₂-C₂₀ alkenyl group, a C₂-C₂₀ alkynyl group, a C₁-C₂₀ alkoxyl group, and a C₆-C₆₀ aryloxy group, and adjacent groups may combine with each other to form a ring, and with the proviso that cases where R¹ to R⁸ are all hydrogen or deuterium are excluded,
Ak is a C₁-C₂₀ alkyl group or a C₃-C₆₀ alicyclic group, and Ak is bonded to any one position among *a, *b, and *c,
a and b are each an integer from 0 to 3, and c is an integer from 0 to 4, and
the aryl group, the arylene group, the fluorenyl group, the fluorenylene group, the heterocyclic group, the alicyclic group, the fused ring, the alkyl group, the alkenyl group, the alkynyl group, the alkoxyl group, the aryloxyl group, and the ring formed by adjacent groups may be each substituted with one or more substituents selected from the group consisting of deuterium, halogen, a silane group unsubstituted or substituted with a C₁-C₂₀ alkyl group or a C₆-C₂₀ aryl group, a phosphine oxide substituted or unsubstituted with a C₁-C₂₀ alkyl group or a C₆-C₂₀ aryl group, a cyano group, a C₁-C₂₀ alkoxy group, a C₆-C₃₀ aryloxy group, a C₁-C₂₀ alkyl group, a C₂-C₂₀ alkenyl group, a C₂-C₂₀ alkynyl group, a C₆-C₃₀ aryl group, a fluorenyl group, a C₂-C₃₀ heterocyclic group comprising at least one heteroatom selected from the group consisting of O, N, S, Si and P, a C₃-C₃₀ alicyclic group, and a fused ring of a C₆-C₃₀ aromatic ring and a C₃-C₃₀ alicyclic group, and wherein the adjacent substituents may be bonded to each other to form a ring, and hydrogen of the substituents may be replaced with deuterium.

2. The compound of claim 1, wherein Formula A is represented by one of Formula A-1 to Formula A-4: 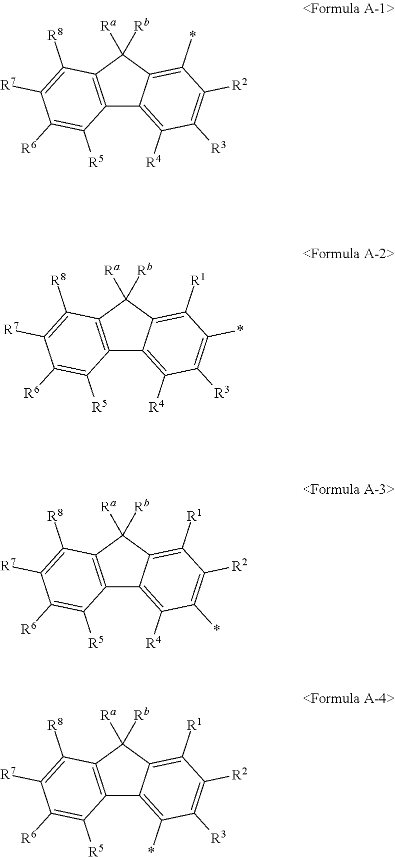
in Formula A-1 to Formula A-4, R¹ to R⁸, R^{a}, R^{b} are the same as defined in claim 1.

3. The compound of claim 1, wherein Formula B is represented by one of Formula B-1 to Formula B-4: 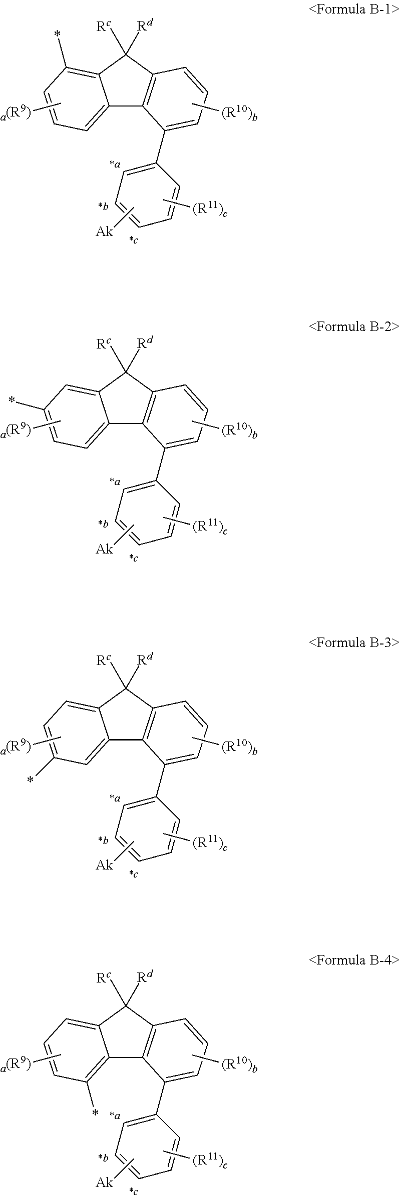
in Formula B-1 to Formula B-4, R⁹ to R¹¹, R^{c}, R^{d}, Ak, a to c are the same as defined in claim 1.

4. The compound of claim 1, wherein Ak is selected from the group consisting of Formula Ak-1 to Formula Ak-8: 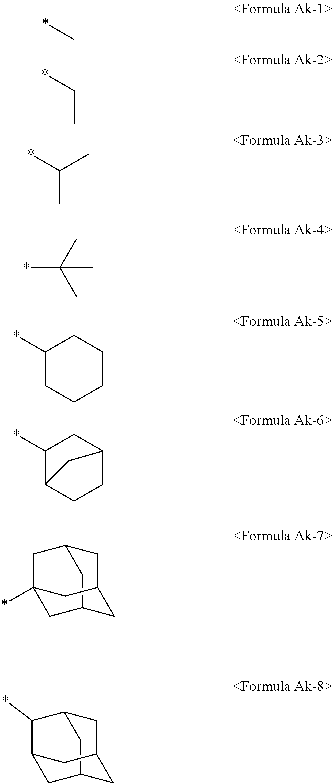
in Formula Ak-1 to Formula Ak-8, the asterisk (*) indicates the position of attachment, and hydrogen may be substituted with deuterium.

5. The compound of claim 1, wherein Ar is selected from the group consisting of Formula Ar-1 to Ar-11: 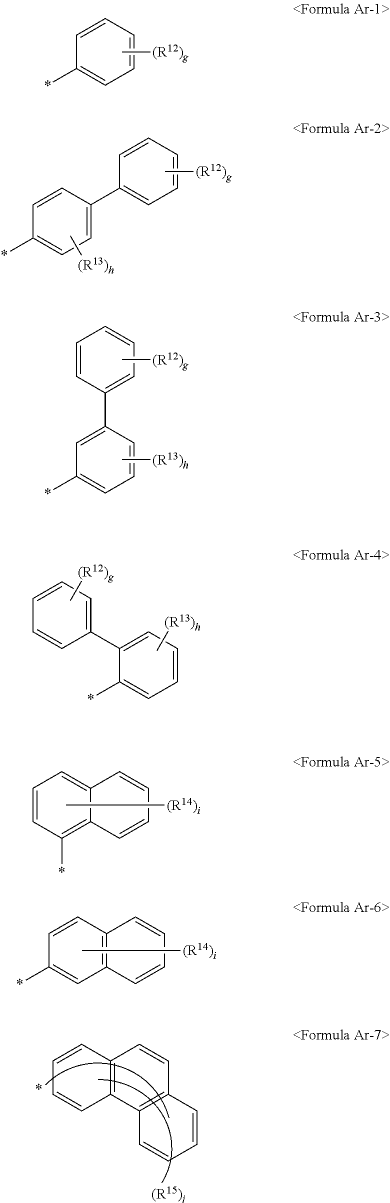 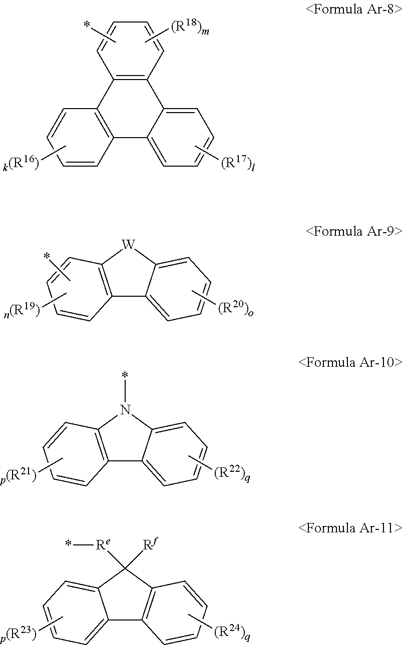
in Formula Ar-1 to Formula Ar-11,
W is O, S, C(R²⁵)(R²⁶) or N(R²⁷),
R¹² to R²⁶ and R^{f} are each independently selected from the group consisting of hydrogen, deuterium, halogen, a silane group unsubstituted or substituted with a C₁-C₂₀ alkyl group or a C₆-C₂₀ aryl group, a phosphine oxide substituted or unsubstituted with a C₁-C₂₀ alkyl group or a C₆-C₂₀ aryl group, a cyano group, a C₁-C₂₀ alkoxy group, a C₆-C₃₀ aryloxy group, a C₁-C₂₀ alkyl group, a C₂-C₂₀ alkenyl group, a C₂-C₂₀ alkynyl group, a C₆-C₃₀ aryl group, a fluorenyl group, a C₂-C₃₀ heterocyclic group comprising at least one heteroatom selected from the group consisting of O, N, S, Si and P, a C₃-C₃₀ alicyclic group, and a fused ring of a C₆-C₃₀ aromatic ring and a C₃-C₃₀ alicyclic group, and wherein the adjacent groups may be bonded to each other to form a ring, and hydrogen of the substituents may be replaced with deuterium,
R^{e} is selected from the group consisting of a single bond, a C₁-C₂₀ alkylene group, a C₆-C₃₀ arylene group, a fluorenylene group, a C₂-C₃₀ heterocyclic group comprising at least one heteroatom selected from the group consisting of O, N, S, Si and P, a C₃-C₃₀ alicyclic group, and a fused ring of a C₆-C₃₀ aromatic ring and a C₃-C₃₀ alicyclic group, and wherein the adjacent groups may be bonded to each other to form a ring, and R^{e} and R^{f} may combine with each other to form a ring,
R²⁷ is selected from the group consisting of a C₁-C₂₀ alkyl group, a C₆-C₃₀ aryl group, a fluorenyl group, a C₂-C₃₀ heterocyclic group comprising at least one heteroatom selected from the group consisting of O, N, S, Si and P, a C₃-C₃₀ alicyclic group, and a fused ring of a C₆-C₃₀ aromatic ring and a C₃-C₃₀ alicyclic group, and
g is an integer from 0 to 5, h, k, l, o, p and q are each an integer from 0 to 4, i is an integer from 0 to 7, j is an integer from 0 to 9; and n and m are each an integer from 0 to 3.

6. The compound of claim 1, wherein at least one of L¹ to L³ is a single bond or is selected from the group consisting of Formula L-1 to Formula L-27: 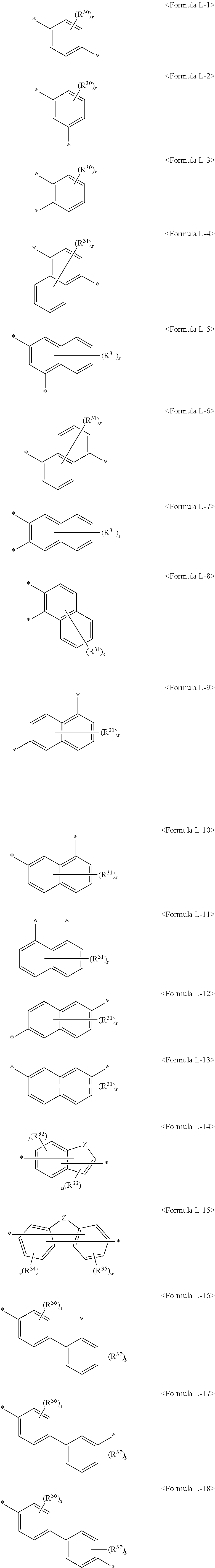 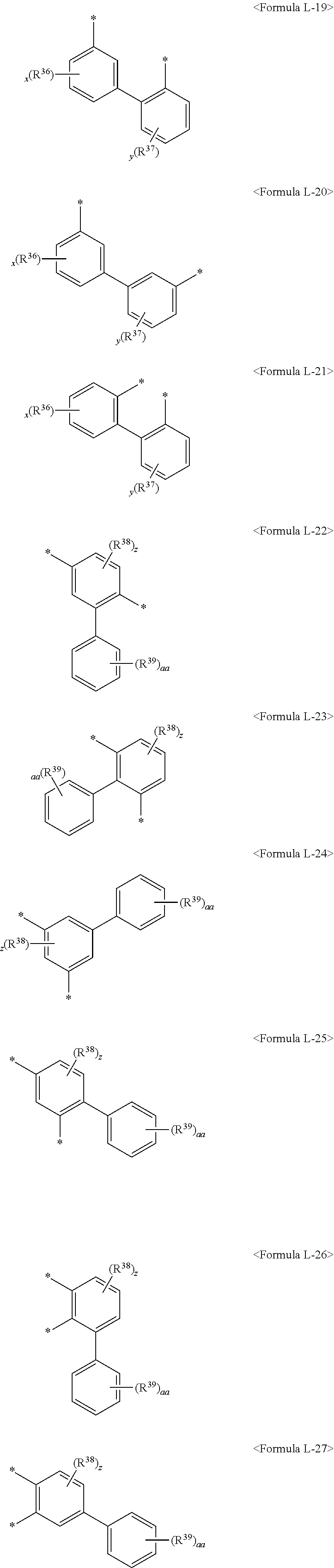
in Formula L-1 to Formula L-27,
Z is O, S, C(R⁴⁰), C(R⁴⁰)(R⁴¹), N or N(R⁴²), with the proviso that when connected through Z, Z is C(R⁴⁰) or N,
R³⁰ to R³⁹, R⁴⁰, R⁴¹ are each independently selected from the group consisting of hydrogen, deuterium, halogen, a silane group unsubstituted or substituted with a C₁-C₂₀ alkyl group or a C₆-C₂₀ aryl group, a phosphine oxide substituted or unsubstituted with a C₁-C₂₀ alkyl group or a C₆-C₂₀ aryl group, a cyano group, a nitro group, a C₁-C₂₀ alkylthio group, a C₁-C₂₀ alkoxy group, a C₆-C₃₀ aryloxy group, a C₆-C₃₀ arylthio, a C₁-C₂₀ alkyl group, a C₂-C₂₀ alkenyl group, a C₂-C₂₀ alkynyl group, a C₆-C₃₀ aryl group, a C₆-C₃₀ aryl group substituted with deuterium, a C₃-C₃₀ alicyclic group, a fused ring of a C₆-C₃₀ aromatic ring and a C₃-C₃₀ alicyclic group, and a C₂-C₃₀ heterocyclic group comprising at least one heteroatom selected from the group consisting of O, N, S, Si and P, and the adjacent groups may combine with each other to form a ring,
R⁴² is selected from the group consisting of a C₁-C₂₀ alkyl group, a C₆-C₃₀ aryl group, a C₆-C₃₀ aryl group substituted with deuterium, a C₃-C₃₀ alicyclic group, a fused ring of a C₆-C₃₀ aromatic ring and a C₃-C₃₀ alicyclic group, and a C₂-C₃₀ heterocyclic group comprising at least one heteroatom selected from the group consisting of O, N, S, Si and P, and
r, t, v, w, x and y are each an integer from 0 to 4, s is an integer from 0 to 6, u is an integer from 0 to 2, z is an integer from 0 to 4, and aa is an integer from 0 to 5.

7. The compound of claim 1, wherein the compound represented by Formula 1 is one of the following compounds:

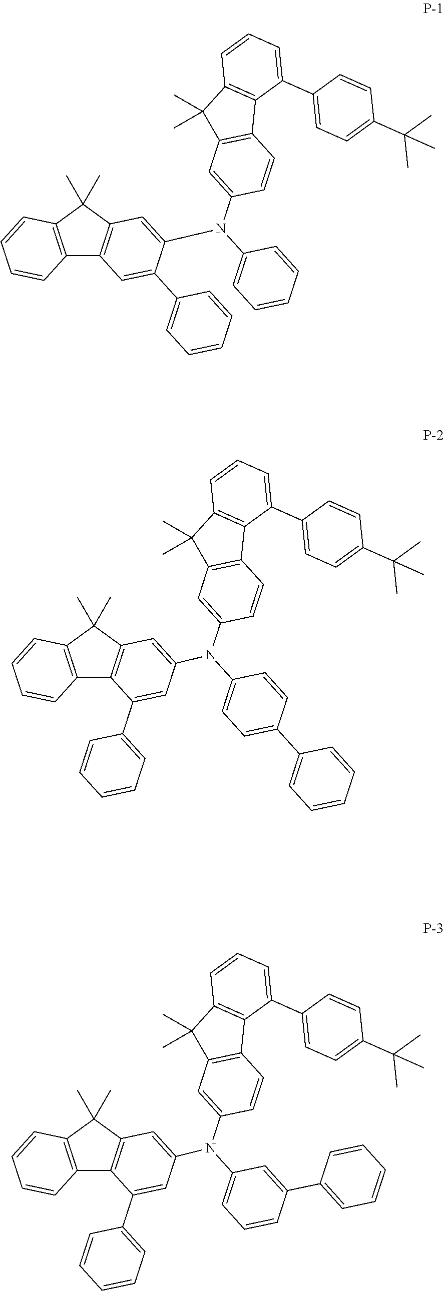 [SMILES: CC(C)(C)c1ccc(-c2cccc3c2-c2ccc(N(c4ccccc4)c4cc5c(cc4-c4ccccc4)-c4ccccc4C5(C)C)cc2C3(C)C)cc1 ; CC(C)(C)c1ccc(-c2cccc3c2-c2ccc(N(c4ccc(-c5ccccc5)cc4)c4cc(-c5ccccc5)c5c(c4)C(C)(C)c4ccccc4-5)cc2C3(C)C)cc1 ; CC(C)(C)c1ccc(-c2cccc3c2-c2ccc(N(c4cccc(-c5ccccc5)c4)c4cc(-c5ccccc5)c5c(c4)C(C)(C)c4ccccc4-5)cc2C3(C)C)cc1]

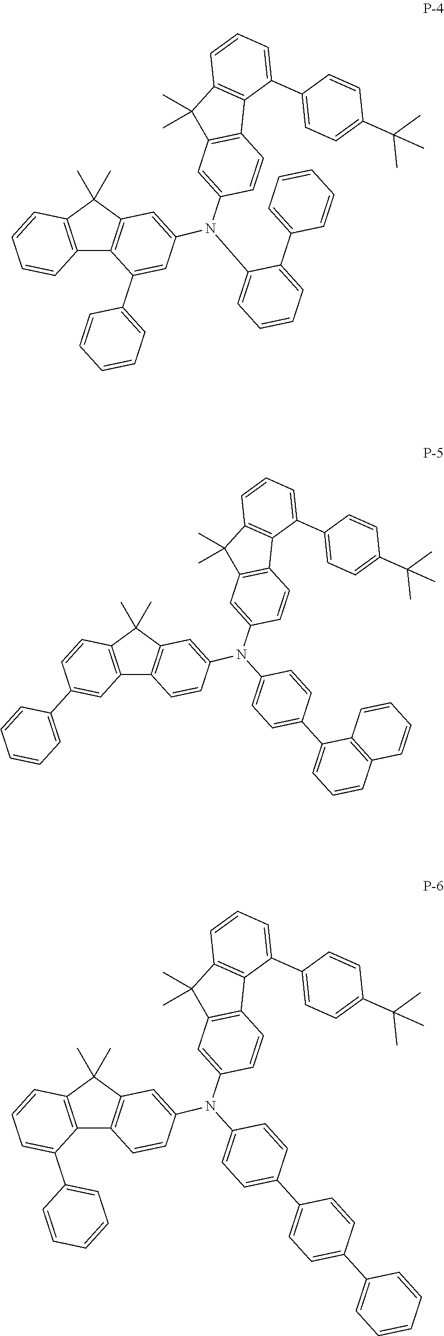 [SMILES: CC(C)(C)c1ccc(-c2cccc3c2-c2ccc(N(c4cc(-c5ccccc5)c5c(c4)C(C)(C)c4ccccc4-5)c4ccccc4-c4ccccc4)cc2C3(C)C)cc1 ; CC(C)(C)c1ccc(-c2cccc3c2-c2ccc(N(c4ccc(-c5cccc6ccccc56)cc4)c4ccc5c(c4)C(C)(C)c4ccc(-c6ccccc6)cc4-5)cc2C3(C)C)cc1 ; CC(C)(C)c1ccc(-c2cccc3c2-c2ccc(N(c4ccc(-c5ccc(-c6ccccc6)cc5)cc4)c4ccc5c(c4)C(C)(C)c4cccc(-c6ccccc6)c4-5)cc2C3(C)C)cc1]

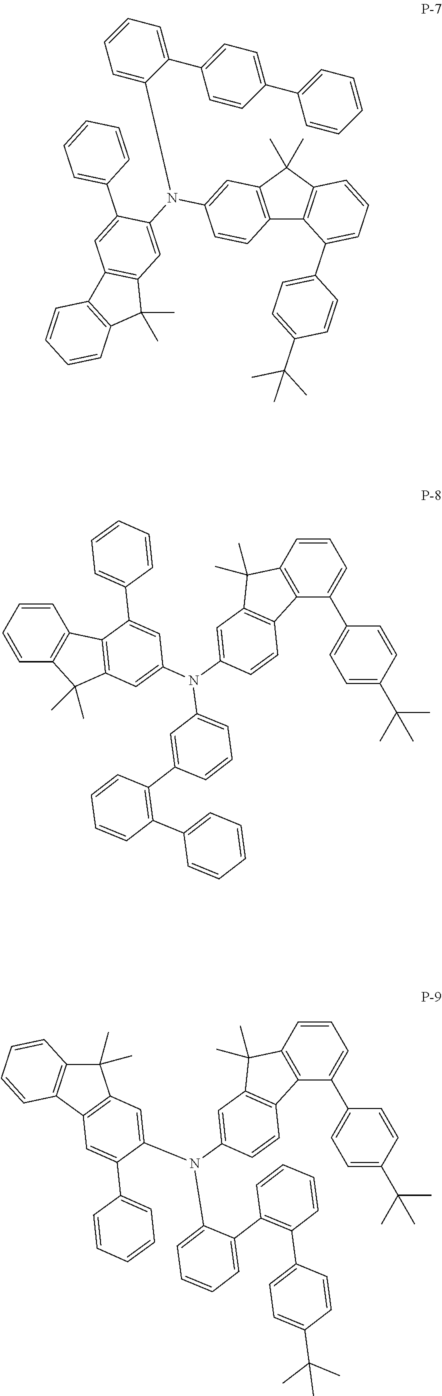 [SMILES: CC(C)(C)c1ccc(-c2cccc3c2-c2ccc(N(c4ccccc4-c4ccc(-c5ccccc5)cc4)c4cc5c(cc4-c4ccccc4)-c4ccccc4C5(C)C)cc2C3(C)C)cc1 ; CC(C)(C)c1ccc(-c2cccc3c2-c2ccc(N(c4cccc(-c5ccccc5-c5ccccc5)c4)c4cc(-c5ccccc5)c5c(c4)C(C)(C)c4ccccc4-5)cc2C3(C)C)cc1 ; CC(C)(C)c1ccc(-c2ccccc2-c2ccccc2N(c2ccc3c(c2)C(C)(C)c2cccc(-c4ccc(C(C)(C)C)cc4)c2-3)c2cc3c(cc2-c2ccccc2)-c2ccccc2C3(C)C)cc1]

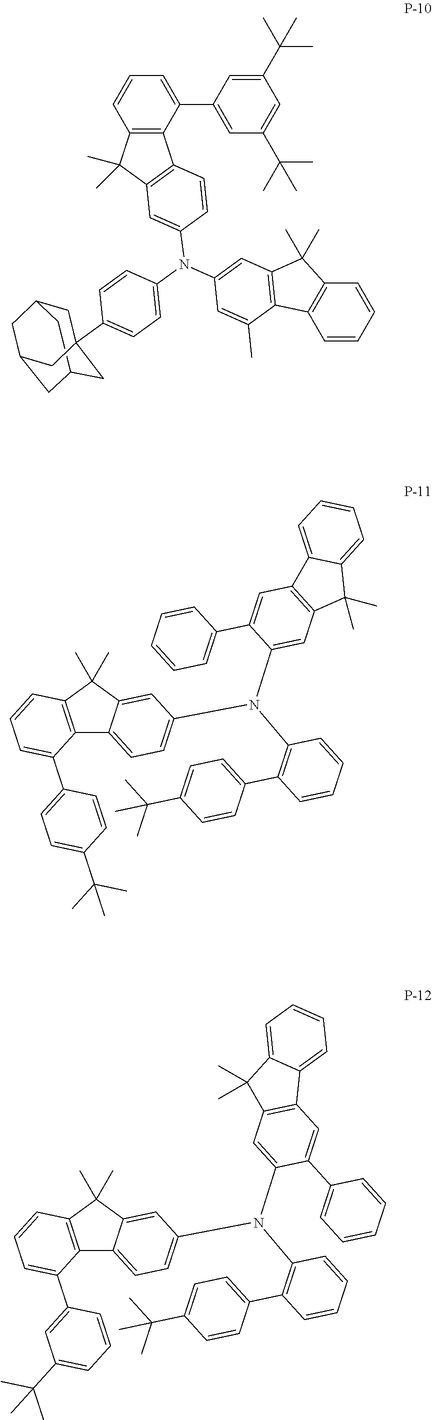

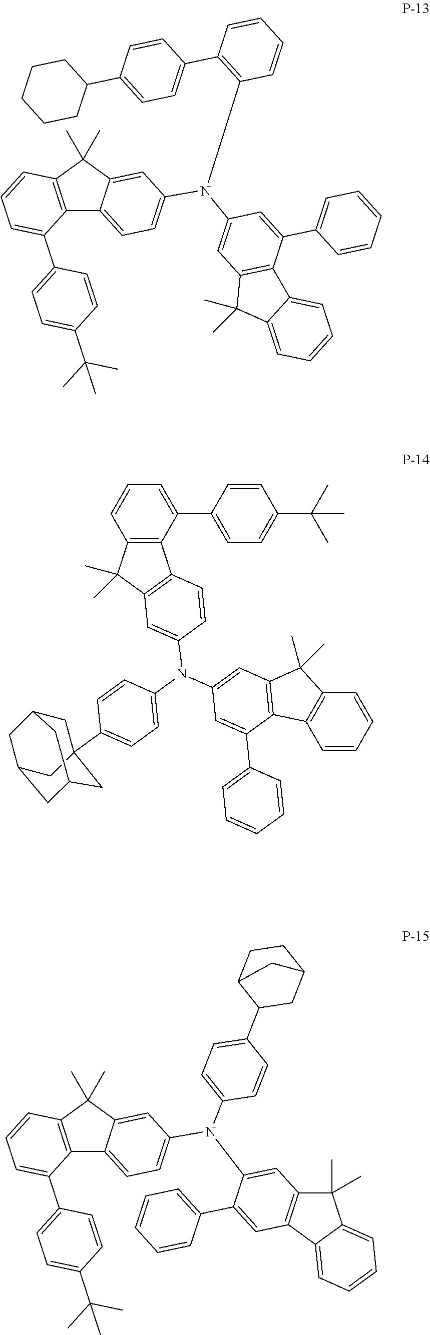 [SMILES: CC(C)(C)c1ccc(-c2cccc3c2-c2ccc(N(c4cc(-c5ccccc5)c5c(c4)C(C)(C)c4ccccc4-5)c4ccccc4-c4ccc(C5CCCCC5)cc4)cc2C3(C)C)cc1 ; CC(C)(C)c1ccc(-c2cccc3c2-c2ccc(N(c4ccc(C56CC7CC(CC(C7)C5)C6)cc4)c4cc(-c5ccccc5)c5c(c4)C(C)(C)c4ccccc4-5)cc2C3(C)C)cc1 ; CC(C)(C)c1ccc(-c2cccc3c2-c2ccc(N(c4ccc(C5CC6CCC5C6)cc4)c4cc5c(cc4-c4ccccc4)-c4ccccc4C5(C)C)cc2C3(C)C)cc1]

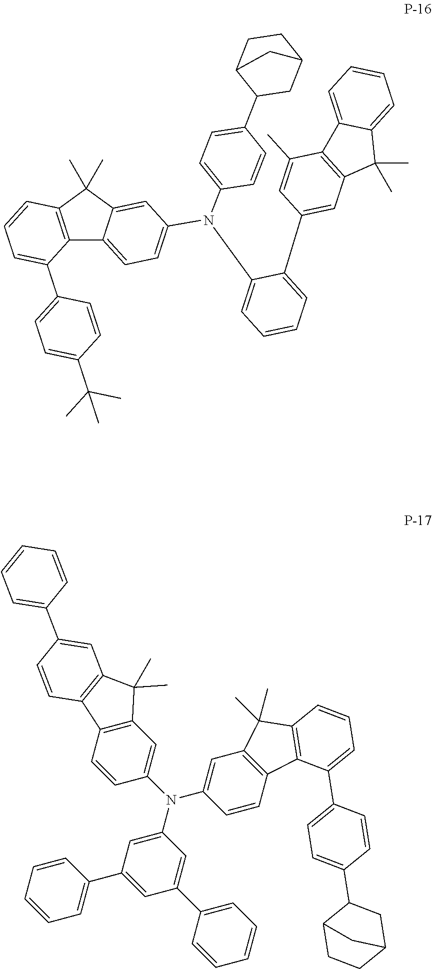 [SMILES: Cc1cc(-c2ccccc2N(c2ccc(C3CC4CCC3C4)cc2)c2ccc3c(c2)C(C)(C)c2cccc(-c4ccc(C(C)(C)C)cc4)c2-3)cc2c1-c1ccccc1C2(C)C ; CC1(C)c2cc(-c3ccccc3)ccc2-c2ccc(N(c3cc(-c4ccccc4)cc(-c4ccccc4)c3)c3ccc4c(c3)C(C)(C)c3cccc(-c5ccc(C6CC7CCC6C7)cc5)c3-4)cc21]

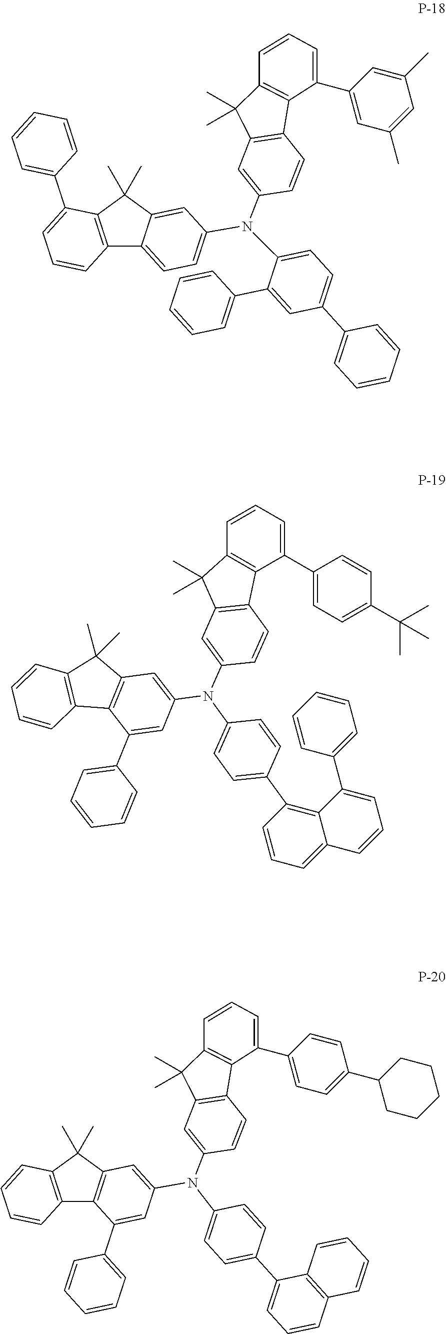 [SMILES: Cc1cc(C)cc(-c2cccc3c2-c2ccc(N(c4ccc5c(c4)C(C)(C)c4c(-c6ccccc6)cccc4-5)c4ccc(-c5ccccc5)cc4-c4ccccc4)cc2C3(C)C)c1 ; CC(C)(C)c1ccc(-c2cccc3c2-c2ccc(N(c4ccc(-c5cccc6cccc(-c7ccccc7)c56)cc4)c4cc(-c5ccccc5)c5c(c4)C(C)(C)c4ccccc4-5)cc2C3(C)C)cc1 ; CC1(C)c2cc(N(c3ccc(-c4cccc5ccccc45)cc3)c3cc(-c4ccccc4)c4c(c3)C(C)(C)c3ccccc3-4)ccc2-c2c(-c3ccc(C4CCCCC4)cc3)cccc21]

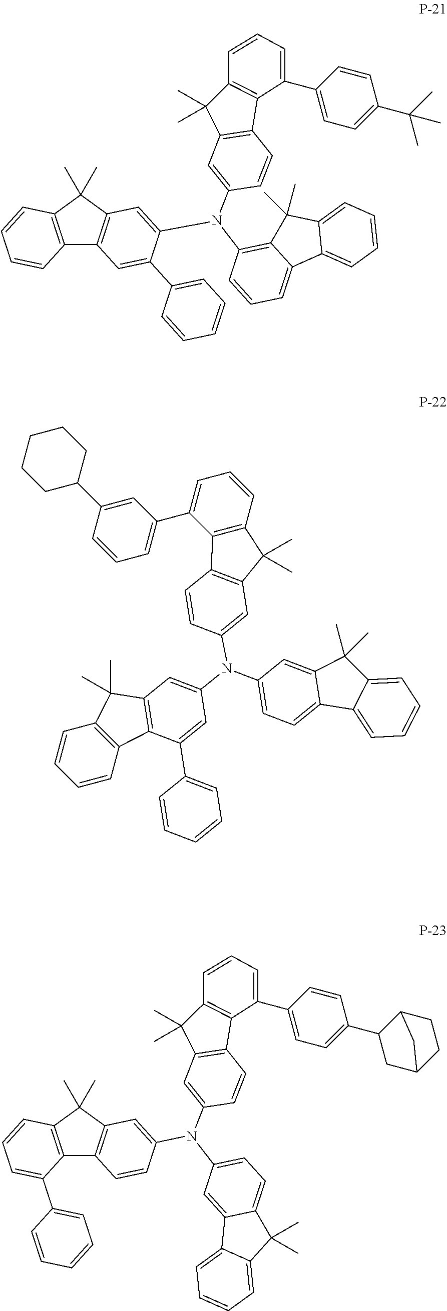 [SMILES: CC(C)(C)c1ccc(-c2cccc3c2-c2ccc(N(c4cc5c(cc4-c4ccccc4)-c4ccccc4C5(C)C)c4cccc5c4C(C)(C)c4ccccc4-5)cc2C3(C)C)cc1 ; CC1(C)c2ccccc2-c2ccc(N(c3ccc4c(c3)C(C)(C)c3cccc(-c5cccc(C6CCCCC6)c5)c3-4)c3cc(-c4ccccc4)c4c(c3)C(C)(C)c3ccccc3-4)cc21 ; CC1(C)c2ccccc2-c2cc(N(c3ccc4c(c3)C(C)(C)c3cccc(-c5ccccc5)c3-4)c3ccc4c(c3)C(C)(C)c3cccc(-c5ccc(C6CC7CCC6C7)cc5)c3-4)ccc21]

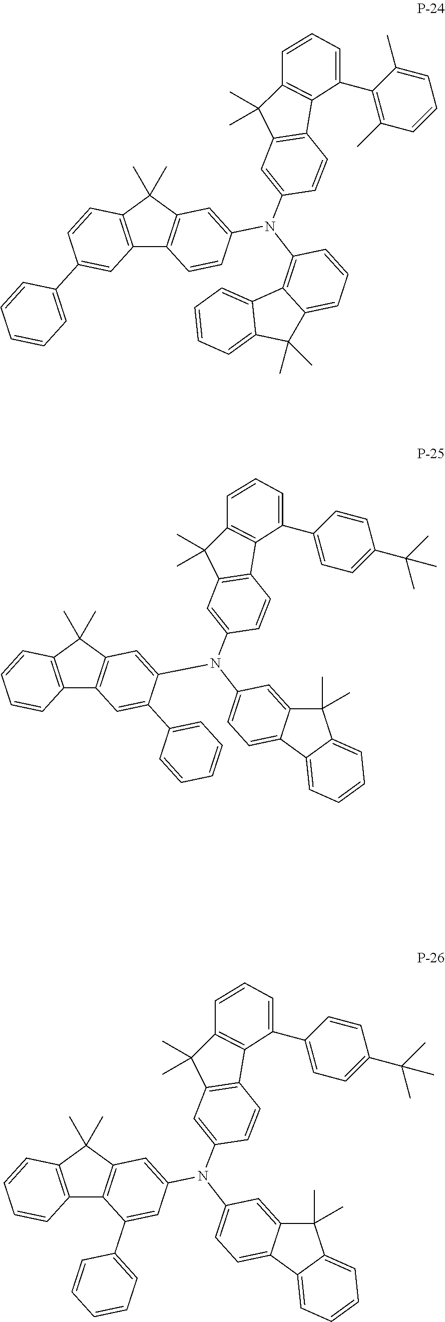 [SMILES: Cc1cccc(C)c1-c1cccc2c1-c1ccc(N(c3ccc4c(c3)C(C)(C)c3ccc(-c5ccccc5)cc3-4)c3cccc4c3-c3ccccc3C4(C)C)cc1C2(C)C ; CC(C)(C)c1ccc(-c2cccc3c2-c2ccc(N(c4ccc5c(c4)C(C)(C)c4ccccc4-5)c4cc5c(cc4-c4ccccc4)-c4ccccc4C5(C)C)cc2C3(C)C)cc1 ; CC(C)(C)c1ccc(-c2cccc3c2-c2ccc(N(c4ccc5c(c4)C(C)(C)c4ccccc4-5)c4cc(-c5ccccc5)c5c(c4)C(C)(C)c4ccccc4-5)cc2C3(C)C)cc1]

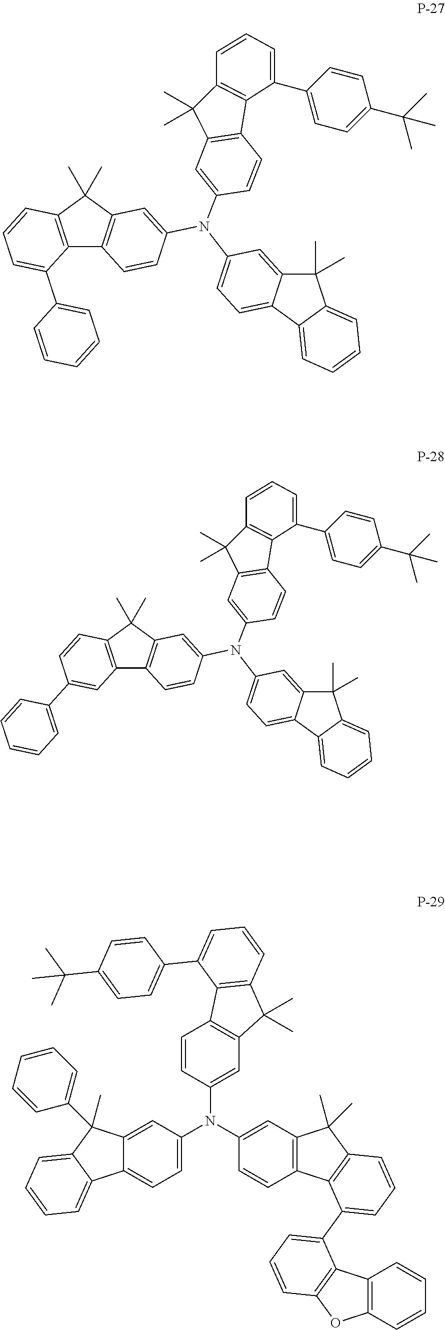 [SMILES: CC(C)(C)c1ccc(-c2cccc3c2-c2ccc(N(c4ccc5c(c4)C(C)(C)c4ccccc4-5)c4ccc5c(c4)C(C)(C)c4cccc(-c6ccccc6)c4-5)cc2C3(C)C)cc1 ; CC(C)(C)c1ccc(-c2cccc3c2-c2ccc(N(c4ccc5c(c4)C(C)(C)c4ccccc4-5)c4ccc5c(c4)C(C)(C)c4ccc(-c6ccccc6)cc4-5)cc2C3(C)C)cc1 ; CC(C)(C)c1ccc(-c2cccc3c2-c2ccc(N(c4ccc5c(c4)C(C)(C)c4cccc(-c6cccc7oc8ccccc8c67)c4-5)c4ccc5c(c4)C(C)(c4ccccc4)c4ccccc4-5)cc2C3(C)C)cc1]

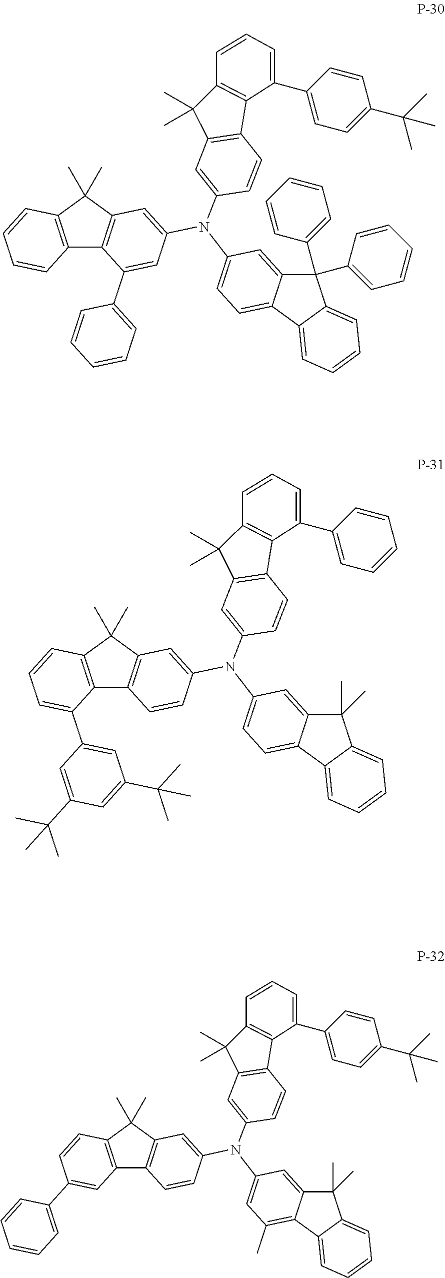

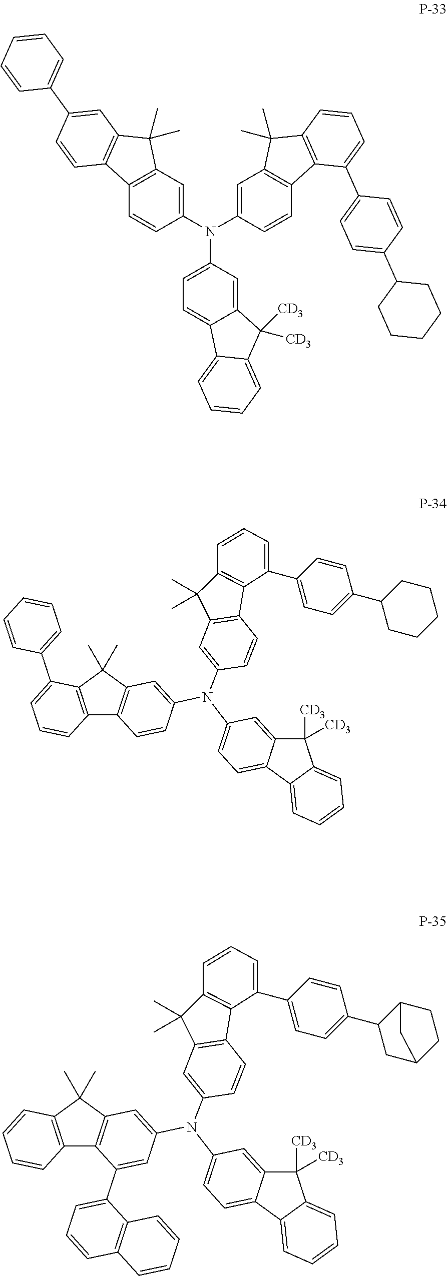 [SMILES: [2H]C([2H])([2H])C1(C([2H])([2H])[2H])c2ccccc2-c2ccc(N(c3ccc4c(c3)C(C)(C)c3cc(-c5ccccc5)ccc3-4)c3ccc4c(c3)C(C)(C)c3cccc(-c5ccc(C6CCCCC6)cc5)c3-4)cc21 ; [2H]C([2H])([2H])C1(C([2H])([2H])[2H])c2ccccc2-c2ccc(N(c3ccc4c(c3)C(C)(C)c3cccc(-c5ccc(C6CCCCC6)cc5)c3-4)c3ccc4c(c3)C(C)(C)c3c(-c5ccccc5)cccc3-4)cc21 ; [2H]C([2H])([2H])C1(C([2H])([2H])[2H])c2ccccc2-c2ccc(N(c3ccc4c(c3)C(C)(C)c3cccc(-c5ccc(C6CC7CCC6C7)cc5)c3-4)c3cc(-c4cccc5ccccc45)c4c(c3)C(C)(C)c3ccccc3-4)cc21]

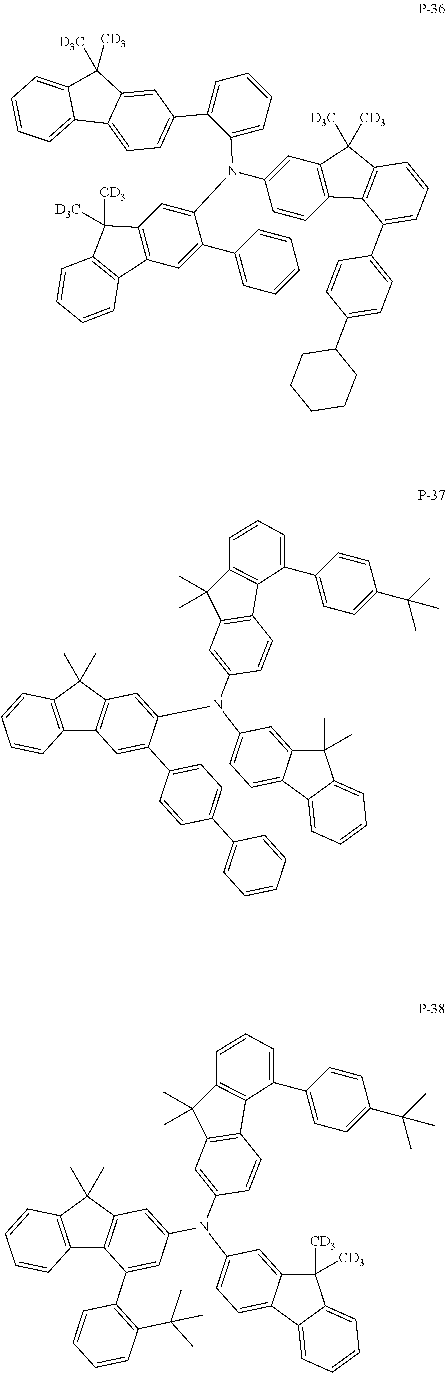

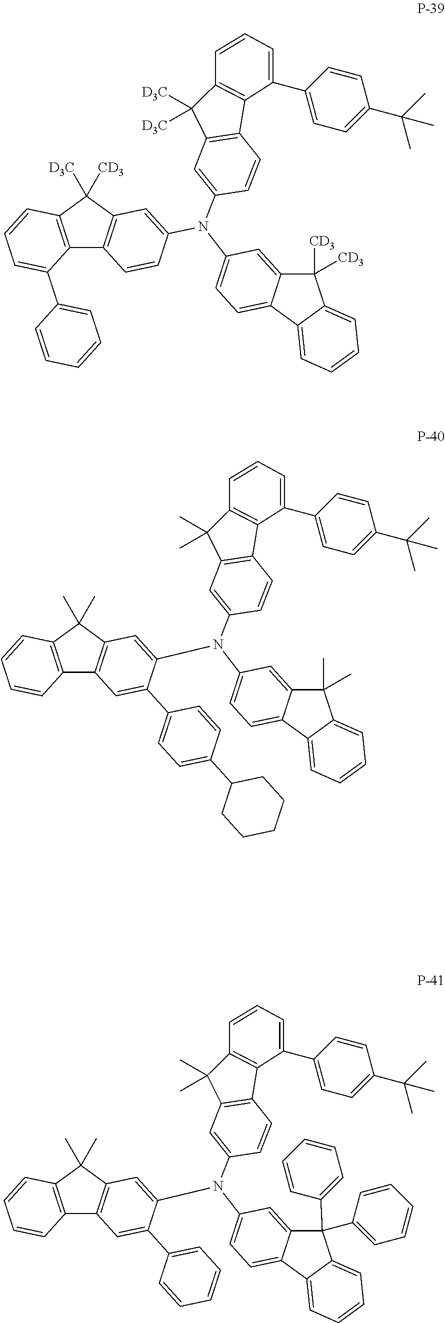 [SMILES: [2H]C([2H])([2H])C1(C([2H])([2H])[2H])c2ccccc2-c2ccc(N(c3ccc4c(c3)C(C([2H])([2H])[2H])(C([2H])([2H])[2H])c3cccc(-c5ccccc5)c3-4)c3ccc4c(c3)C(C([2H])([2H])[2H])(C([2H])([2H])[2H])c3cccc(-c5ccc(C(C)(C)C)cc5)c3-4)cc21 ; CC(C)(C)c1ccc(-c2cccc3c2-c2ccc(N(c4ccc5c(c4)C(C)(C)c4ccccc4-5)c4cc5c(cc4-c4ccc(C6CCCCC6)cc4)-c4ccccc4C5(C)C)cc2C3(C)C)cc1 ; CC(C)(C)c1ccc(-c2cccc3c2-c2ccc(N(c4ccc5c(c4)C(c4ccccc4)(c4ccccc4)c4ccccc4-5)c4cc5c(cc4-c4ccccc4)-c4ccccc4C5(C)C)cc2C3(C)C)cc1]

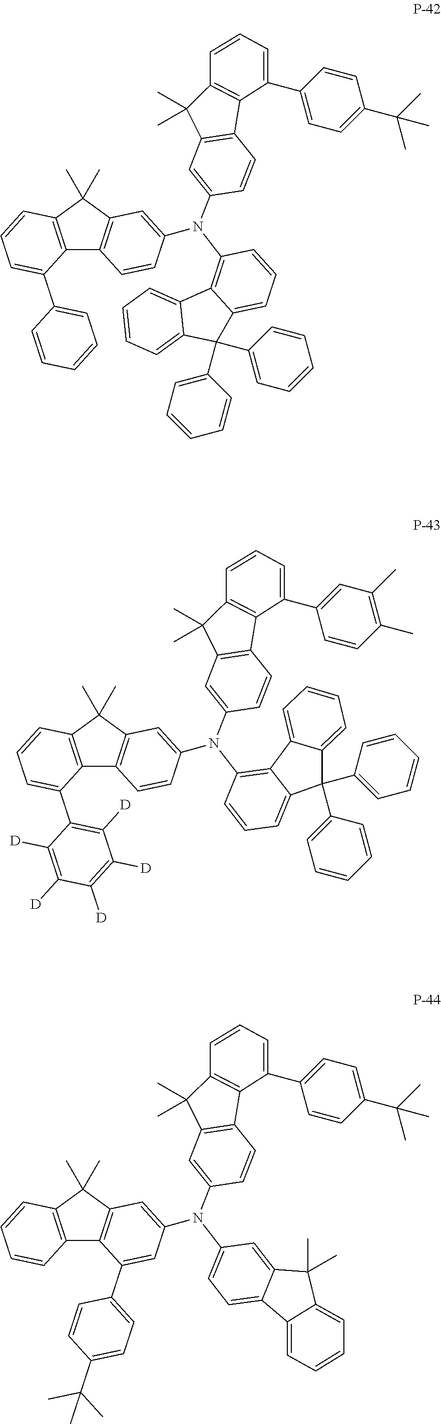 [SMILES: CC(C)(C)c1ccc(-c2cccc3c2-c2ccc(N(c4ccc5c(c4)C(C)(C)c4cccc(-c6ccccc6)c4-5)c4cccc5c4-c4ccccc4C5(c4ccccc4)c4ccccc4)cc2C3(C)C)cc1 ; [2H]c1c([2H])c([2H])c(-c2cccc3c2-c2ccc(N(c4ccc5c(c4)C(C)(C)c4cccc(-c6ccc(C)c(C)c6)c4-5)c4cccc5c4-c4ccccc4C5(c4ccccc4)c4ccccc4)cc2C3(C)C)c([2H])c1[2H] ; CC(C)(C)c1ccc(-c2cccc3c2-c2ccc(N(c4ccc5c(c4)C(C)(C)c4ccccc4-5)c4cc(-c5ccc(C(C)(C)C)cc5)c5c(c4)C(C)(C)c4ccccc4-5)cc2C3(C)C)cc1]

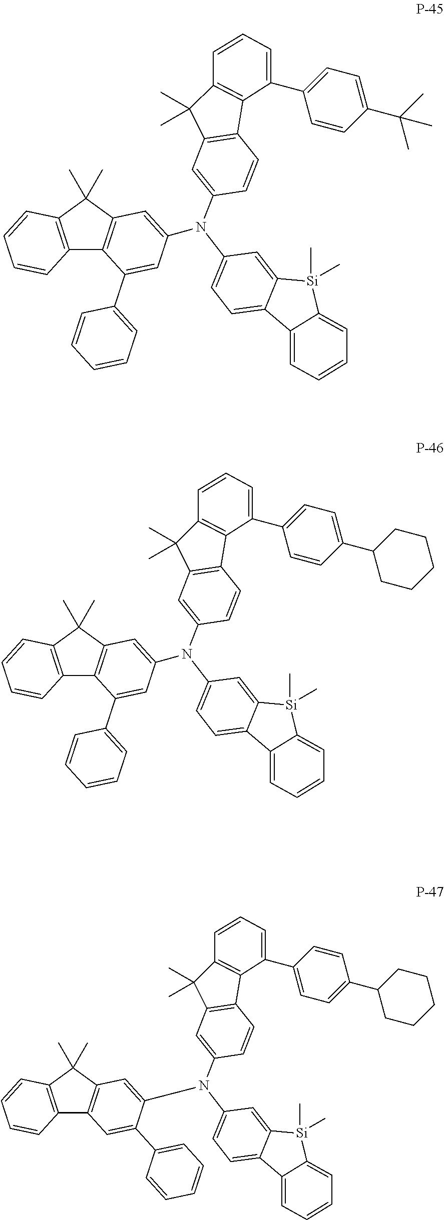 [SMILES: CC(C)(C)c1ccc(-c2cccc3c2-c2ccc(N(c4cc(-c5ccccc5)c5c(c4)C(C)(C)c4ccccc4-5)c4ccc5c(c4)[Si](C)(C)c4ccccc4-5)cc2C3(C)C)cc1 ; CC1(C)c2cc(N(c3cc(-c4ccccc4)c4c(c3)C(C)(C)c3ccccc3-4)c3ccc4c(c3)[Si](C)(C)c3ccccc3-4)ccc2-c2c(-c3ccc(C4CCCCC4)cc3)cccc21 ; CC1(C)c2ccccc2-c2cc(-c3ccccc3)c(N(c3ccc4c(c3)C(C)(C)c3cccc(-c5ccc(C6CCCCC6)cc5)c3-4)c3ccc4c(c3)[Si](C)(C)c3ccccc3-4)cc21]

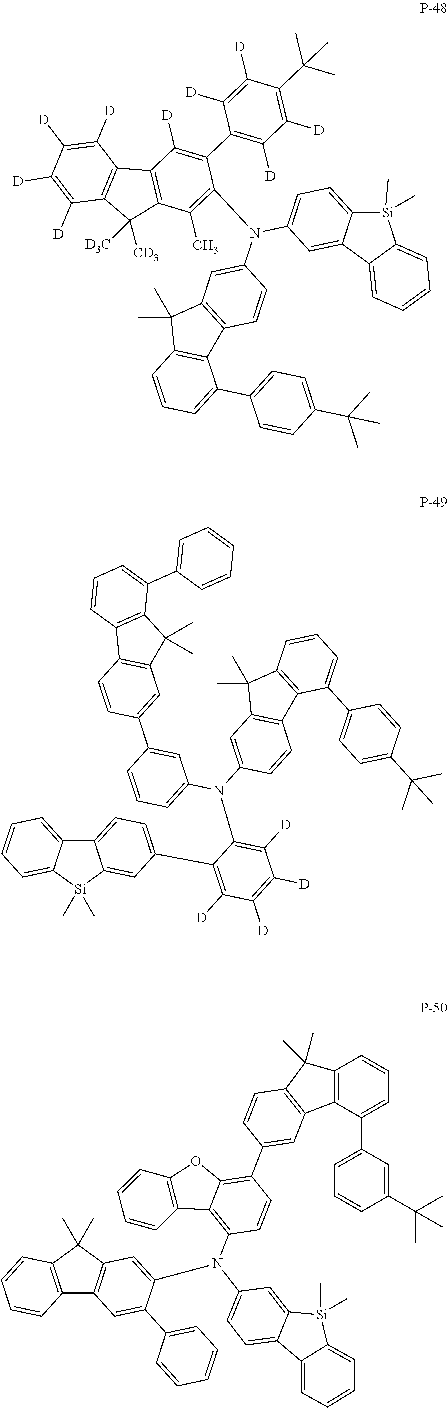

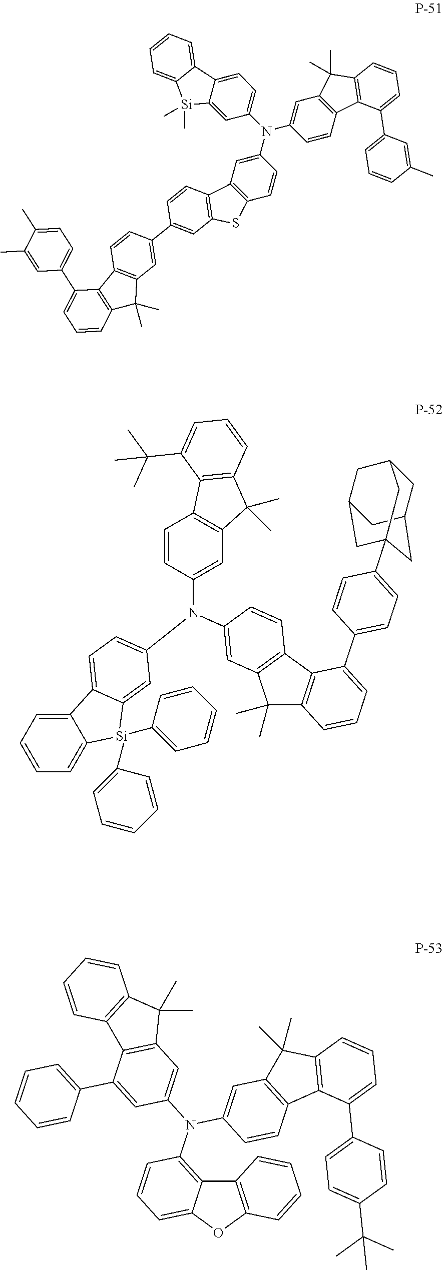

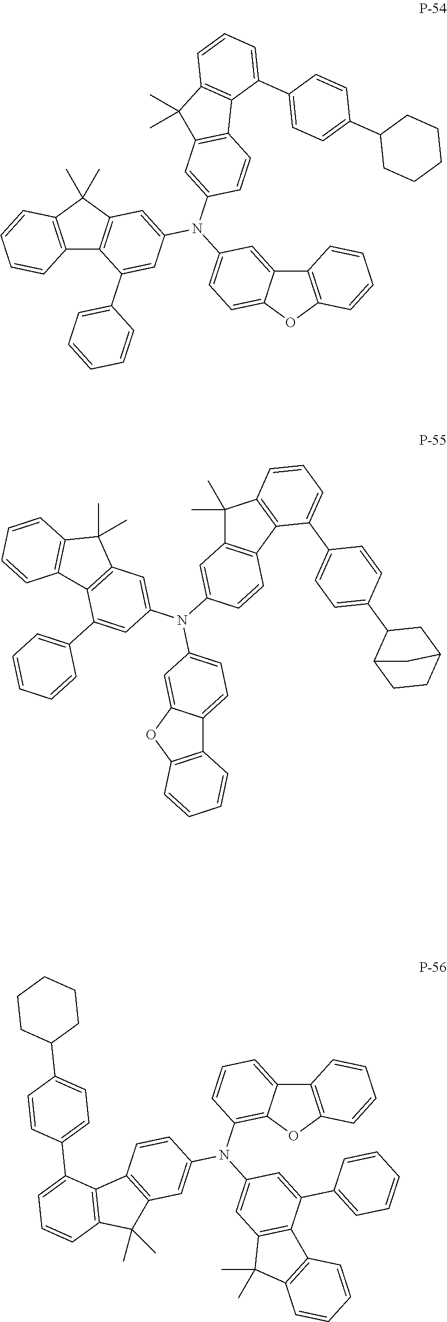 [SMILES: CC1(C)c2cc(N(c3cc(-c4ccccc4)c4c(c3)C(C)(C)c3ccccc3-4)c3ccc4oc5ccccc5c4c3)ccc2-c2c(-c3ccc(C4CCCCC4)cc3)cccc21 ; CC1(C)c2cc(N(c3cc(-c4ccccc4)c4c(c3)C(C)(C)c3ccccc3-4)c3ccc4c(c3)oc3ccccc34)ccc2-c2c(-c3ccc(C4CC5CCC4C5)cc3)cccc21 ; CC1(C)c2cc(N(c3cc(-c4ccccc4)c4c(c3)C(C)(C)c3ccccc3-4)c3cccc4c3oc3ccccc34)ccc2-c2c(-c3ccc(C4CCCCC4)cc3)cccc21]

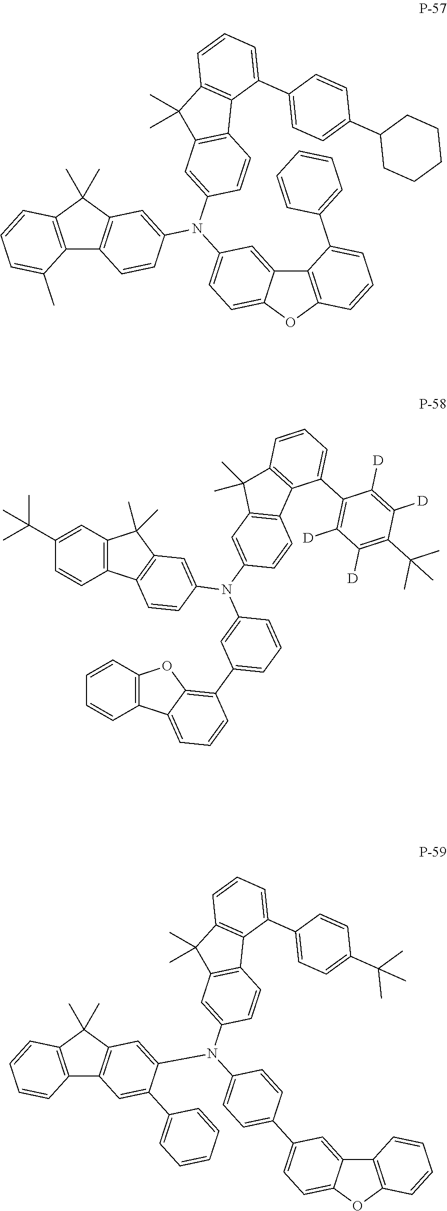 [SMILES: Cc1cccc2c1-c1ccc(N(c3ccc4c(c3)C(C)(C)c3cccc(-c5ccc(C6CCCCC6)cc5)c3-4)c3ccc4oc5cccc(-c6ccccc6)c5c4c3)cc1C2(C)C ; [2H]c1c([2H])c(C(C)(C)C)c([2H])c([2H])c1-c1cccc2c1-c1ccc(N(c3cccc(-c4cccc5c4oc4ccccc45)c3)c3ccc4c(c3)C(C)(C)c3cc(C(C)(C)C)ccc3-4)cc1C2(C)C ; CC(C)(C)c1ccc(-c2cccc3c2-c2ccc(N(c4ccc(-c5ccc6oc7ccccc7c6c5)cc4)c4cc5c(cc4-c4ccccc4)-c4ccccc4C5(C)C)cc2C3(C)C)cc1]

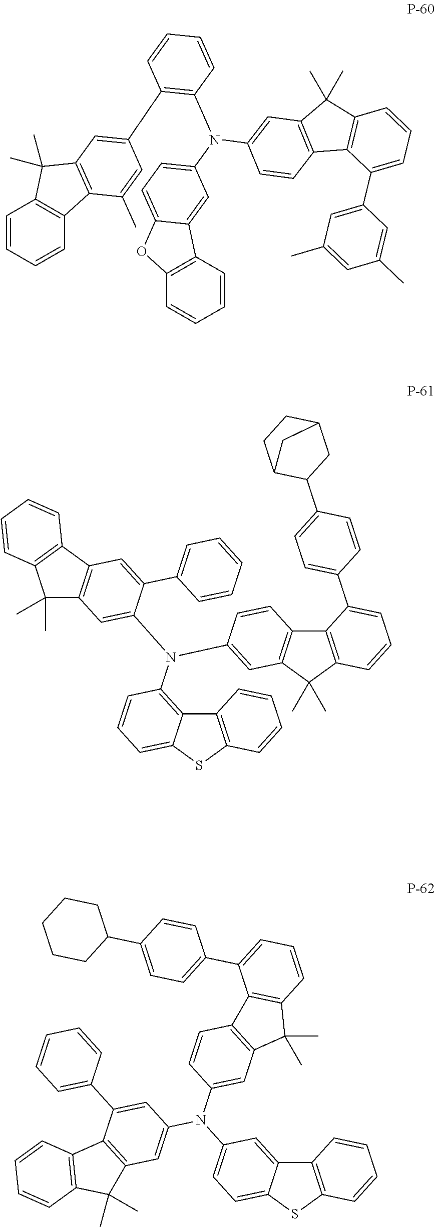 [SMILES: Cc1cc(C)cc(-c2cccc3c2-c2ccc(N(c4ccc5oc6ccccc6c5c4)c4ccccc4-c4cc(C)c5c(c4)C(C)(C)c4ccccc4-5)cc2C3(C)C)c1 ; CC1(C)c2ccccc2-c2cc(-c3ccccc3)c(N(c3ccc4c(c3)C(C)(C)c3cccc(-c5ccc(C6CC7CCC6C7)cc5)c3-4)c3cccc4sc5ccccc5c34)cc21 ; CC1(C)c2cc(N(c3cc(-c4ccccc4)c4c(c3)C(C)(C)c3ccccc3-4)c3ccc4sc5ccccc5c4c3)ccc2-c2c(-c3ccc(C4CCCCC4)cc3)cccc21]

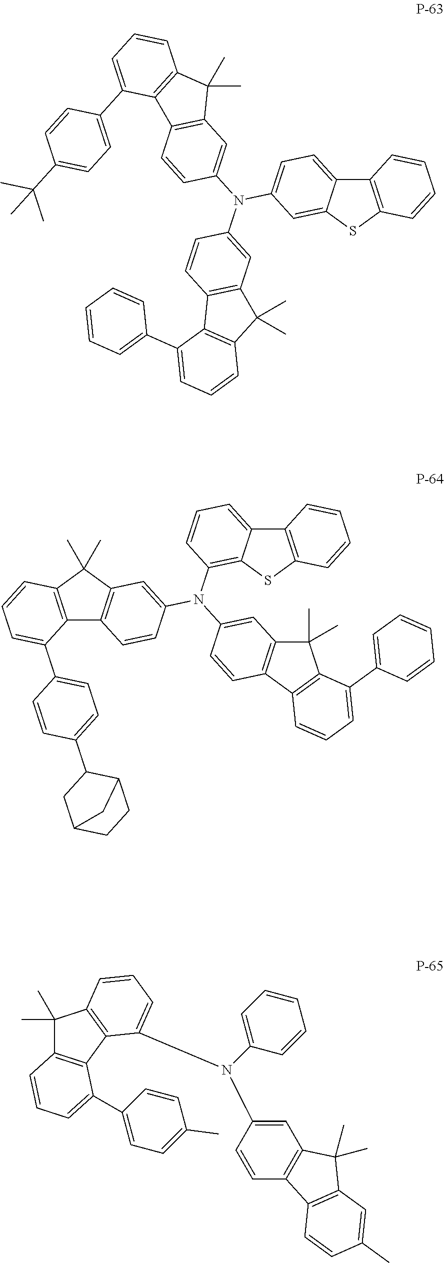

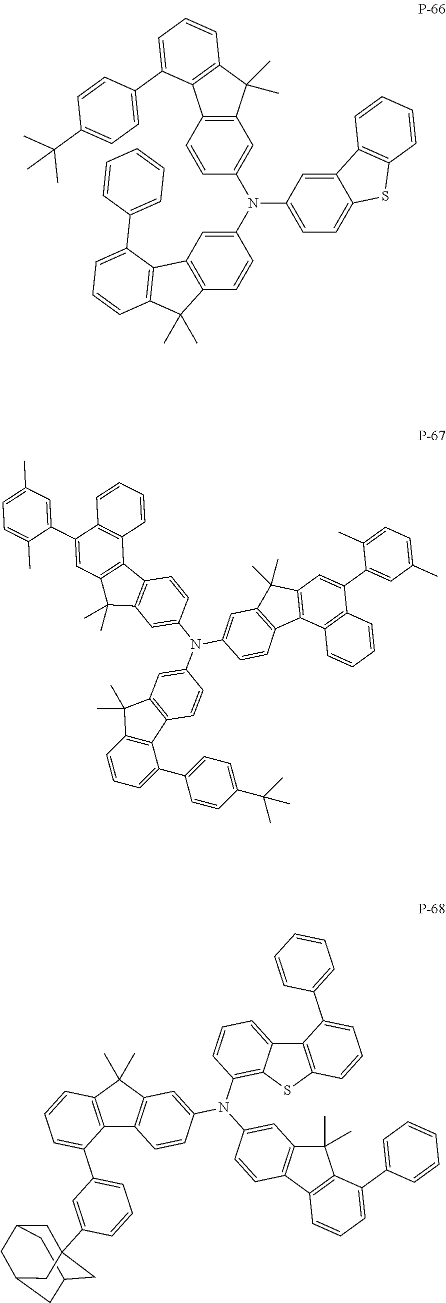 [SMILES: CC(C)(C)c1ccc(-c2cccc3c2-c2ccc(N(c4ccc5c(c4)-c4c(-c6ccccc6)cccc4C5(C)C)c4ccc5sc6ccccc6c5c4)cc2C3(C)C)cc1 ; Cc1ccc(C)c(-c2cc3c(c4ccccc24)-c2ccc(N(c4ccc5c(c4)C(C)(C)c4cccc(-c6ccc(C(C)(C)C)cc6)c4-5)c4ccc5c(c4)C(C)(C)c4cc(-c6cc(C)ccc6C)c6ccccc6c4-5)cc2C3(C)C)c1 ; CC1(C)c2cc(N(c3ccc4c(c3)C(C)(C)c3c(-c5ccccc5)cccc3-4)c3cccc4c3sc3cccc(-c5ccccc5)c34)ccc2-c2c(-c3cccc(C45CC6CC(CC(C6)C4)C5)c3)cccc21]

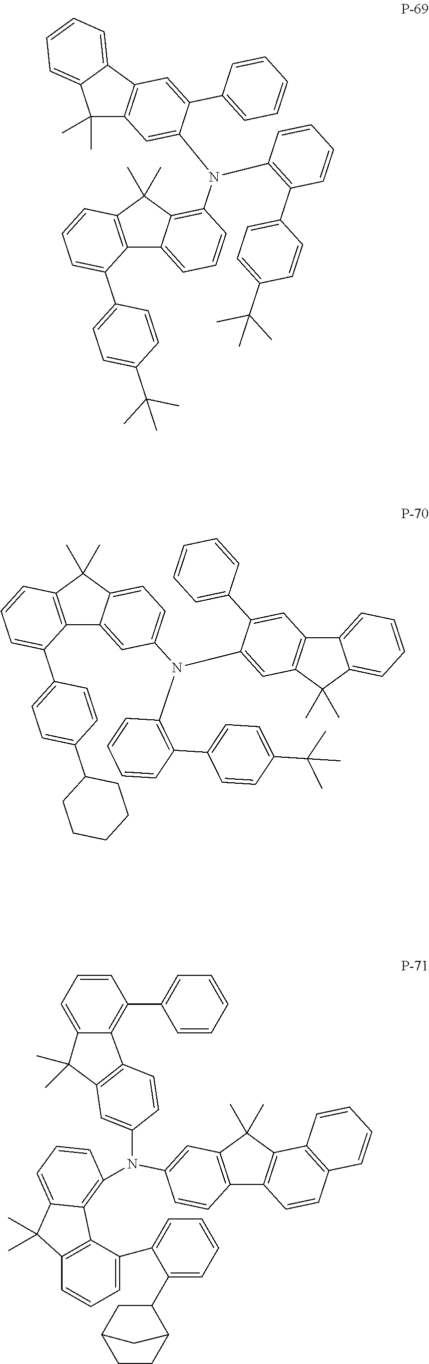 [SMILES: CC(C)(C)c1ccc(-c2ccccc2N(c2cc3c(cc2-c2ccccc2)-c2ccccc2C3(C)C)c2cccc3c2C(C)(C)c2cccc(-c4ccc(C(C)(C)C)cc4)c2-3)cc1 ; CC(C)(C)c1ccc(-c2ccccc2N(c2ccc3c(c2)-c2c(-c4ccc(C5CCCCC5)cc4)cccc2C3(C)C)c2cc3c(cc2-c2ccccc2)-c2ccccc2C3(C)C)cc1 ; CC1(C)c2cc(N(c3ccc4c(c3)C(C)(C)c3c-4ccc4ccccc34)c3cccc4c3-c3c(-c5ccccc5C5CC6CCC5C6)cccc3C4(C)C)ccc2-c2c(-c3ccccc3)cccc21]

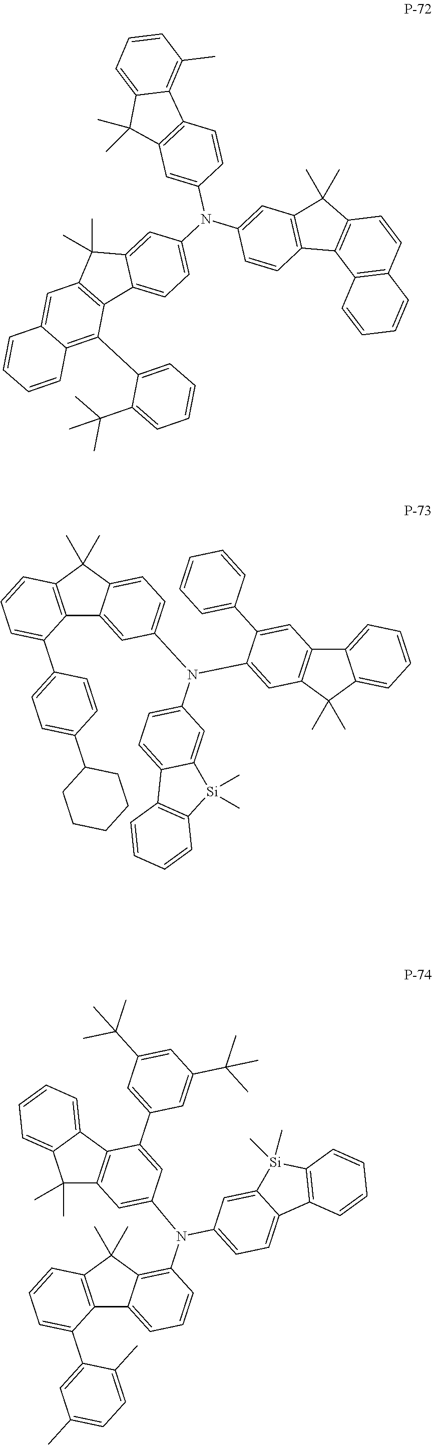 [SMILES: Cc1cccc2c1-c1ccc(N(c3ccc4c(c3)C(C)(C)c3cc5ccccc5c(-c5ccccc5C(C)(C)C)c3-4)c3ccc4c(c3)C(C)(C)c3ccc5ccccc5c3-4)cc1C2(C)C ; CC1(C)c2ccccc2-c2cc(-c3ccccc3)c(N(c3ccc4c(c3)-c3c(-c5ccc(C6CCCCC6)cc5)cccc3C4(C)C)c3ccc4c(c3)[Si](C)(C)c3ccccc3-4)cc21 ; Cc1ccc(C)c(-c2cccc3c2-c2cccc(N(c4cc(-c5cc(C(C)(C)C)cc(C(C)(C)C)c5)c5c(c4)C(C)(C)c4ccccc4-5)c4ccc5c(c4)[Si](C)(C)c4ccccc4-5)c2C3(C)C)c1]

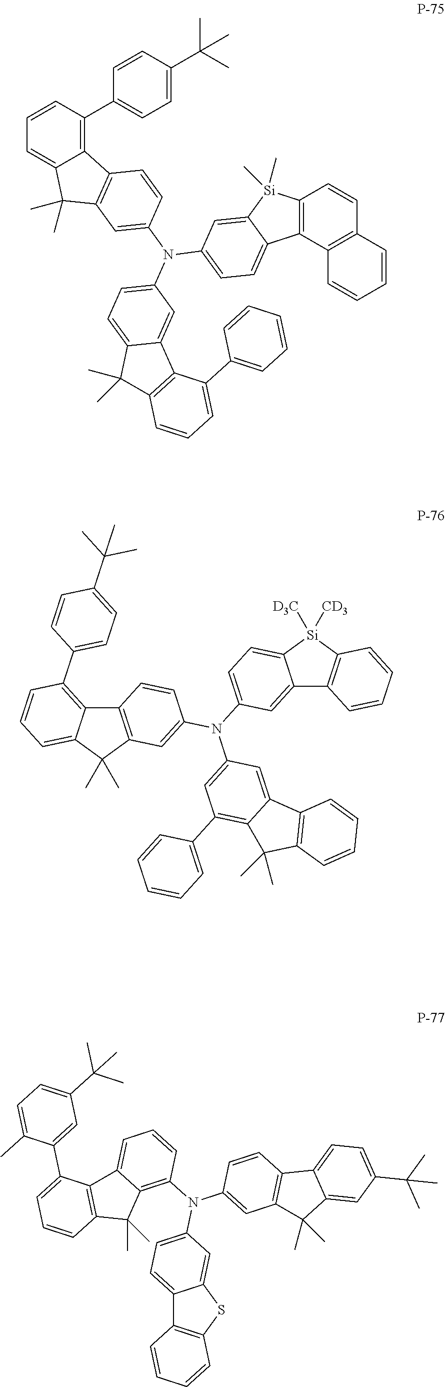 [SMILES: CC(C)(C)c1ccc(-c2cccc3c2-c2ccc(N(c4ccc5c(c4)-c4c(-c6ccccc6)cccc4C5(C)C)c4ccc5c(c4)[Si](C)(C)c4ccc6ccccc6c4-5)cc2C3(C)C)cc1 ; [2H]C([2H])([2H])[Si]1(C([2H])([2H])[2H])c2ccccc2-c2cc(N(c3cc(-c4ccccc4)c4c(c3)-c3ccccc3C4(C)C)c3ccc4c(c3)C(C)(C)c3cccc(-c5ccc(C(C)(C)C)cc5)c3-4)ccc21 ; Cc1ccc(C(C)(C)C)cc1-c1cccc2c1-c1cccc(N(c3ccc4c(c3)C(C)(C)c3cc(C(C)(C)C)ccc3-4)c3ccc4c(c3)sc3ccccc34)c1C2(C)C]

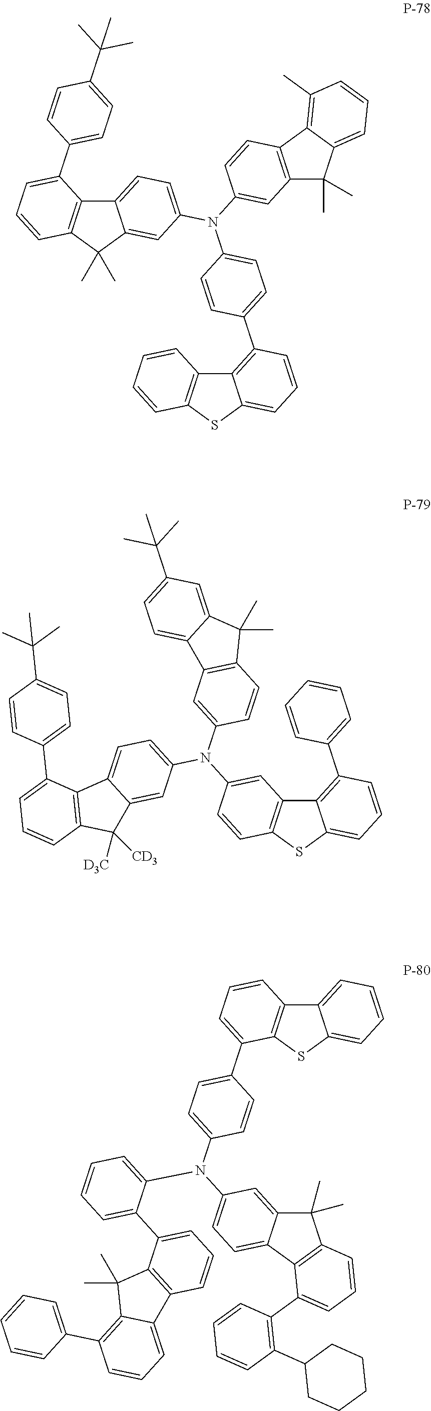 [SMILES: Cc1cccc2c1-c1ccc(N(c3ccc(-c4cccc5sc6ccccc6c45)cc3)c3ccc4c(c3)C(C)(C)c3cccc(-c5ccc(C(C)(C)C)cc5)c3-4)cc1C2(C)C ; [2H]C([2H])([2H])C1(C([2H])([2H])[2H])c2cc(N(c3ccc4c(c3)-c3ccc(C(C)(C)C)cc3C4(C)C)c3ccc4sc5cccc(-c6ccccc6)c5c4c3)ccc2-c2c(-c3ccc(C(C)(C)C)cc3)cccc21 ; CC1(C)c2cc(N(c3ccc(-c4cccc5c4sc4ccccc45)cc3)c3ccccc3-c3cccc4c3C(C)(C)c3c(-c5ccccc5)cccc3-4)ccc2-c2c(-c3ccccc3C3CCCCC3)cccc21]

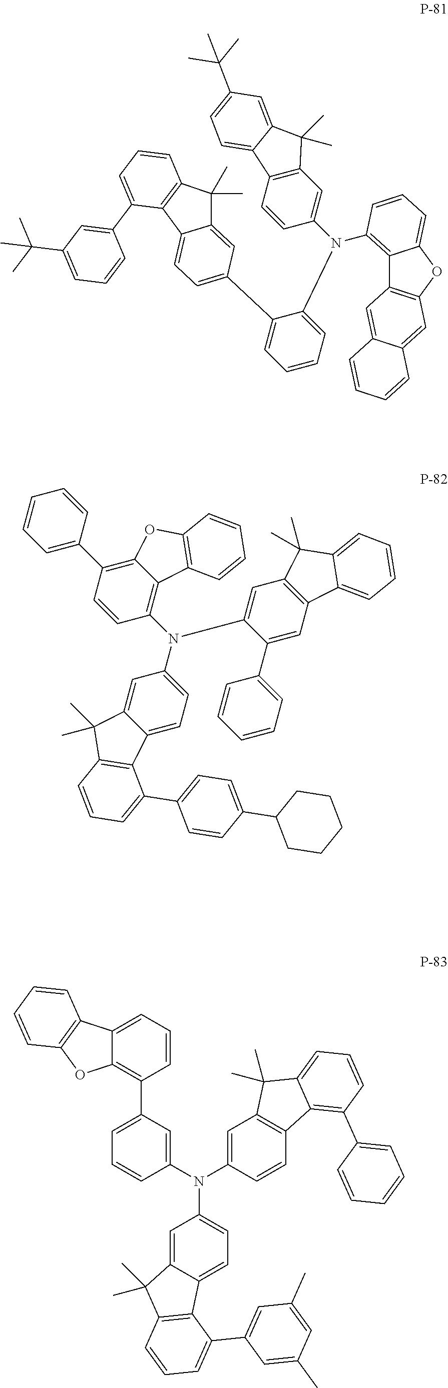 [SMILES: CC(C)(C)c1cccc(-c2cccc3c2-c2ccc(-c4ccccc4N(c4ccc5c(c4)C(C)(C)c4cc(C(C)(C)C)ccc4-5)c4cccc5oc6cc7ccccc7cc6c45)cc2C3(C)C)c1 ; CC1(C)c2ccccc2-c2cc(-c3ccccc3)c(N(c3ccc4c(c3)C(C)(C)c3cccc(-c5ccc(C6CCCCC6)cc5)c3-4)c3ccc(-c4ccccc4)c4oc5ccccc5c34)cc21 ; Cc1cc(C)cc(-c2cccc3c2-c2ccc(N(c4cccc(-c5cccc6c5oc5ccccc56)c4)c4ccc5c(c4)C(C)(C)c4cccc(-c6ccccc6)c4-5)cc2C3(C)C)c1]

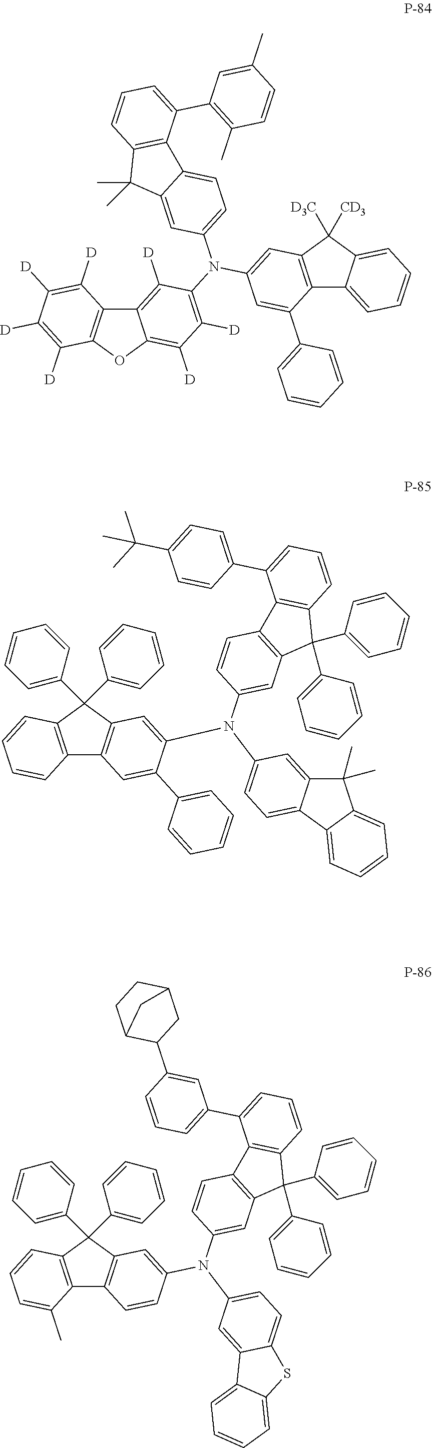 [SMILES: [2H]c1c([2H])c([2H])c2c(oc3c([2H])c([2H])c(N(c4ccc5c(c4)C(C)(C)c4cccc(-c6cc(C)ccc6C)c4-5)c4cc(-c5ccccc5)c5c(c4)C(C([2H])([2H])[2H])(C([2H])([2H])[2H])c4ccccc4-5)c([2H])c32)c1[2H] ; CC(C)(C)c1ccc(-c2cccc3c2-c2ccc(N(c4ccc5c(c4)C(C)(C)c4ccccc4-5)c4cc5c(cc4-c4ccccc4)-c4ccccc4C5(c4ccccc4)c4ccccc4)cc2C3(c2ccccc2)c2ccccc2)cc1 ; Cc1cccc2c1-c1ccc(N(c3ccc4c(c3)C(c3ccccc3)(c3ccccc3)c3cccc(-c5cccc(C6CC7CCC6C7)c5)c3-4)c3ccc4sc5ccccc5c4c3)cc1C2(c1ccccc1)c1ccccc1]

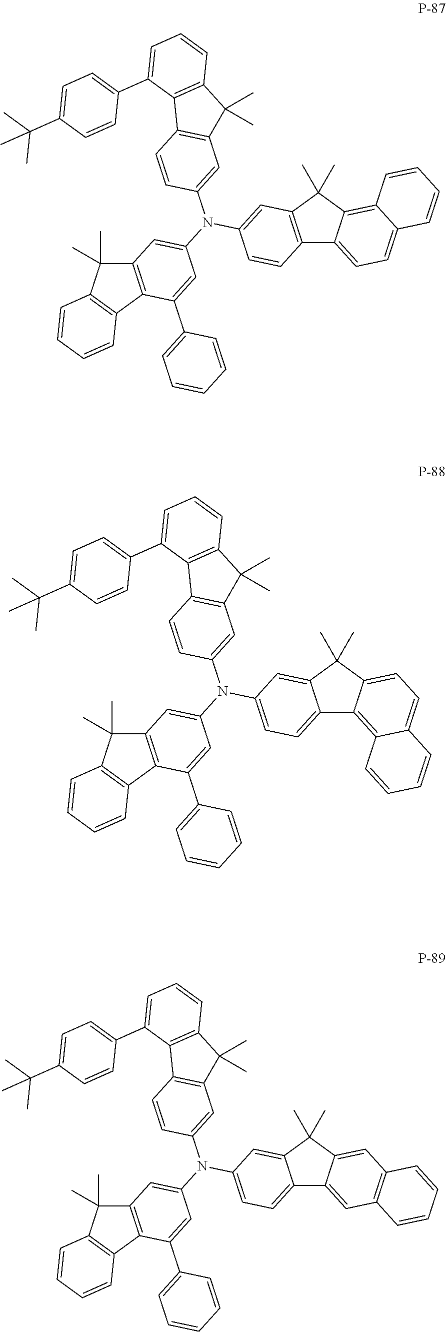 [SMILES: CC(C)(C)c1ccc(-c2cccc3c2-c2ccc(N(c4ccc5c(c4)C(C)(C)c4c-5ccc5ccccc45)c4cc(-c5ccccc5)c5c(c4)C(C)(C)c4ccccc4-5)cc2C3(C)C)cc1 ; CC(C)(C)c1ccc(-c2cccc3c2-c2ccc(N(c4ccc5c(c4)C(C)(C)c4ccc6ccccc6c4-5)c4cc(-c5ccccc5)c5c(c4)C(C)(C)c4ccccc4-5)cc2C3(C)C)cc1 ; CC(C)(C)c1ccc(-c2cccc3c2-c2ccc(N(c4ccc5c(c4)C(C)(C)c4cc6ccccc6cc4-5)c4cc(-c5ccccc5)c5c(c4)C(C)(C)c4ccccc4-5)cc2C3(C)C)cc1]

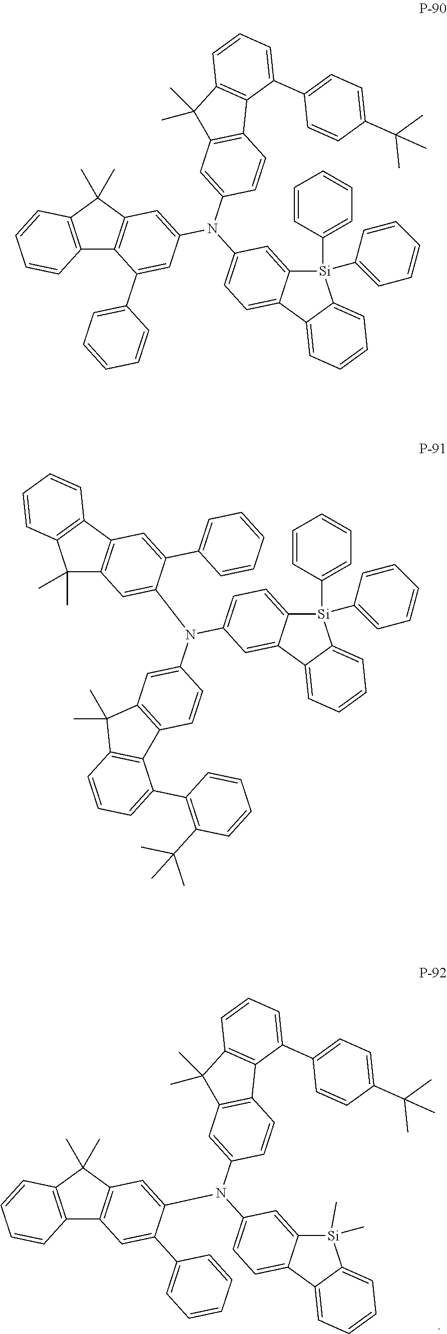 [SMILES: CC(C)(C)c1ccc(-c2cccc3c2-c2ccc(N(c4cc(-c5ccccc5)c5c(c4)C(C)(C)c4ccccc4-5)c4ccc5c(c4)[Si](c4ccccc4)(c4ccccc4)c4ccccc4-5)cc2C3(C)C)cc1 ; CC(C)(C)c1ccccc1-c1cccc2c1-c1ccc(N(c3ccc4c(c3)-c3ccccc3[Si]4(c3ccccc3)c3ccccc3)c3cc4c(cc3-c3ccccc3)-c3ccccc3C4(C)C)cc1C2(C)C ; CC(C)(C)c1ccc(-c2cccc3c2-c2ccc(N(c4ccc5c(c4)[Si](C)(C)c4ccccc4-5)c4cc5c(cc4-c4ccccc4)-c4ccccc4C5(C)C)cc2C3(C)C)cc1]

8. An organic electronic device comprising a first electrode, a second electrode, and an organic layer between the first electrode and the second electrode, wherein the organic layer comprises the compound of Formula 1 of claim 1.

9. The organic electronic device of claim 8, wherein the organic layer further comprises a light-emitting layer between the first electrode and the second electrode, and a light-emitting auxiliary layer between the light-emitting layer and the first electrode, and the light-emitting auxiliary layer comprises the compound.

10. The organic electronic device of claim 8, wherein the organic layer comprises two or more stacks, and the two or more stacks each comprise a hole transport layer, a light-emitting layer and an electron transport layer formed sequentially on the first electrode.

11. An electronic apparatus comprising a display device and a control unit for driving the display device, wherein the display device comprises the organic electronic device of claim 8.

12. The electronic apparatus of claim 11, wherein the electronic apparatus is a wired or wireless communication terminal.

13. The electronic apparatus of claim 11, wherein the wired or wireless communication terminal is a mobile communication terminal, a navigation device, a game console, a TV, or a computer.

14. A method for recovering a compound represented by Formula 1 comprising:
a step of depositing a material of organic layer comprising a compound represented by Formula 1 of claim 1;
a step of recovering the material of organic layer attached to the deposition equipment; and
a step of purifying the recovered material of an organic layer to obtain a compound represented by Formula 1 having a purity of 99.9% or higher.
